SEQUENCE LISTING (21 sequences)

SEQ ID NO: 1 - protein, 4 aa, Artificial Sequence
  Artificial protein
FFRK

SEQ ID NO: 2 - protein, 8 aa, Artificial Sequence
  Artificial protein
NVYDFNLL

SEQ ID NO: 3 - protein, 19 aa, Artificial Sequence
  Artificial protein
AAAHSLSNVYDFNLLLERD

SEQ ID NO: 4 - protein, 8 aa, Artificial Sequence
  Artificial protein
NVFDFNNL

SEQ ID NO: 5 - protein, 14 aa, Artificial Sequence
  Artificial protein
AAASTNVFDFNNLS

SEQ ID NO: 6 - protein, 18 aa, Artificial Sequence
  Artificial protein
DNQKDIYYITGESINAVS

SEQ ID NO: 7 - protein, 16 aa, Artificial Sequence
  Artificial protein
AAALTSALLNVDNLIQ

SEQ ID NO: 8 - protein, 11 aa, Artificial Sequence
  Artificial protein
STNVFDFNNLS

SEQ ID NO: 9 - protein, 8 aa, Artificial Sequence
  Artificial protein
SALLNVDN

SEQ ID NO: 10 - protein, 7 aa, Artificial Sequence
  Artificial protein
SIINFEK

SEQ ID NO: 11 - protein, 31 aa, Artificial Sequence
  Artificial protein
KISQAVHAAHAEINEAGRESIINFEKLTEWT

SEQ ID NO: 12 - protein, 16 aa, Artificial Sequence
  Artificial protein
HSLSNVYDFNLLLERD

SEQ ID NO: 13 - protein, 8 aa, Artificial Sequence
  Artificial protein
EIYIFTNI

SEQ ID NO: 14 - protein, 14 aa, Artificial Sequence
  Artificial protein
LSNYVDFNLLLERD

SEQ ID NO: 15 - protein, 6 aa, Artificial Sequence
  Artificial protein
AAVNVY

SEQ ID NO: 16 - protein, 4 aa, Artificial Sequence
  Artificial protein
FKFL

SEQ ID NO: 17 - protein, 4 aa, Artificial Sequence
  Artificial protein
GFLG

SEQ ID NO: 18 - protein, 9 aa, Artificial Sequence
  Artificial protein
ILNSGLLAV

SEQ ID NO: 19 - protein, 13 aa, Artificial Sequence
  Artificial protein
TKILNSGLLAVVG

SEQ ID NO: 20 - protein, 17 aa, Artificial Sequence
  Artificial protein
HSLSILNSGLLAVLERD

SEQ ID NO: 21 - protein, 221 aa, Plasmodium falciparum ortholog PF3D7_1338200
MTNTSNELKHYNVKGKKKVLVPVNAKKTINKKYFGRKVASKKKYVVQRKLRKSIEVGKVAIILTGKHMGKRCIITKILNSGLLAVVGPYEINGVPLKRVDSRYLVVTSTNIFNFENIAKLKDDFLNYAQDIDDDSFIKTLEIKKKQKKLLKNKNEALFMNNVIDKIKEIRKEDPKVQKLEGIQKDIGSLLKPEILKNKVFAHYLKSKFTLRNDMVLHKMKF

(19) United States
(12) Patent Application Publication
(10) Pub. No.: US 2022/0233668 A1
(43) Pub. Date: Jul. 28, 2022

(54) GLYCOPEPTIDE VACCINE

(71) Applicants: VICTORIA LINK LTD., Wellinton (NZ); MAILCORP BIODISCOVERIES LIMIETD, Wellinto (NZ); VICTORIA UNIVERSITY OF WELLINGTON, Wellingto (NZ)

(72) Inventors: Dale Ian GODFREY, Parkville (AU); William Ross HEATH, Parkville (AU); Lauren Eelise HOLZ, Parkville (AU); Ian Francis HERMANS, Aro Valley (NZ); Gavin Frank PAINTER, Waterloo (NZ); Regan James ANDERSON, Waterloo (NZ); Benjamin Jason COPTON, Waterloo (NZ); Shivali Ashwin GULAB, Astirua, NY (US)

(21) Appl. No.: 17/609,641

(22) PCT Filed: May 8, 2020
(86) PCT No.: PCT/NZ2020/050048

§ 371 Date: Nov. 8, 2021

Related U.S. Application Data
(60) Provisional application No. 62/846,327, filed on May 10, 2019.

(51) Int. Cl. A61K 39/015; A61P 33/06

(52) U.S. Cl. CPC A61K 39/015; A61P 33/06

(57) ABSTRACT

The present invention generally relates to a glycopeptide conjugate compound of Formula (I):, as described herein, compositions comprising the conjugate compound and to the use of such a compound to as a vaccine.

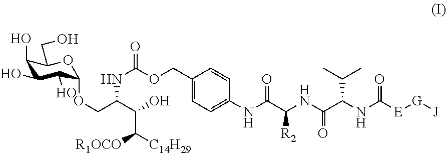

### 1. FIELD OF THE INVENTION

The present invention relates generally to glycolipid-peptide conjugates of Formula I and the use of these conjugates as vaccines to prevent or to reduce the incidence of malaria in a subject.

### 2. BACKGROUND

Malaria is a highly prevalent parasitic disease, accounting for approximately 216 million infections and over 445,000 deaths per annum in 2016. The disease reduces the GDP of badly affected countries by billions every year and causes serious physical and mental impairment of affected children.

The disease is caused by Plasmodium spp. which is transmitted via female Anopheles mosquitoes. Following a bite from an infected mosquito, immature parasites (sporozoites) injected into the skin circulate via the blood to the liver where they infect hepatocytes. Over approximately one week in humans, or two days in mice, the sporozoites mature and replicate within hepatocytes. The sporozites are then released into the blood as merozoites able to infect and destroy red blood cells and cause disease.

The complexity of both the malaria parasite, and the resultant immune response make development of a malaria vaccine very difficult. Although vaccination with radiation attenuated sporozoites (RAS) was demonstrated to elicit sterile protection against malaria 30 years ago, to date there is only a single malaria vaccine currently approved for use in humans.

The RTS,S vaccine (Mosquirix) is a recombinant protein-based vaccine, approved in 2015. While Mosquirix is considered to be the most advanced vaccine candidate in trials, it requires four injections, and has a relatively low efficacy. In view of this low efficacy, the World Health Organization is carrying out large scale pilot trials of the vaccine before recommending its use in infants.

Accordingly, there is a great need in the art for new malaria vaccines. It is an object of the invention to provide glycolipid-peptide conjugates that are useful as malarial vaccines and/or to provide methods of using such conjugates for reducing the incidence of malarial infection and/or for preventing malaria and/or to at least provide the public with a useful choice.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### 3. SUMMARY OF INVENTION

In one aspect the invention relates to a compound of Formula I:

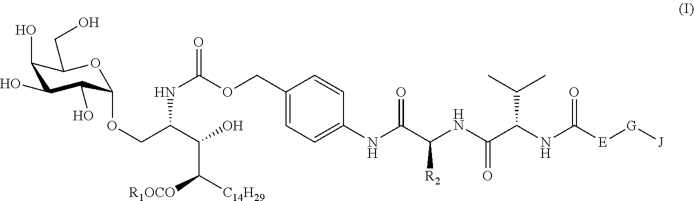

wherein
R₁ is (C₁₇-C₂₅)alkyl,
R₂ is the side-chain for alanine or citrulline,
E is a linker selected from S or Ox

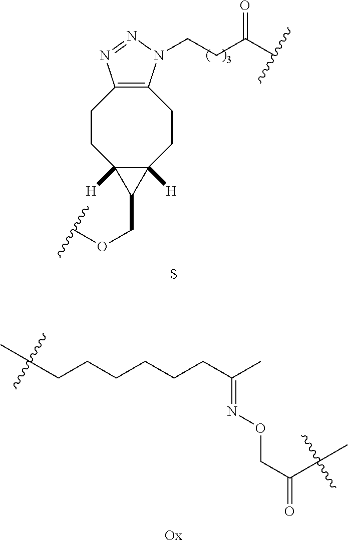

G is absent or is an amino acid sequence selected from the group consisting of FFRK (SEQ ID NO: 1), FKFL (SEQ ID NO: 16) and GFLG (SEQ ID NO: 17); and
J is a peptide antigen.

In one embodiment, the compound of Formula I is a compound of Formula V.S.G.J:

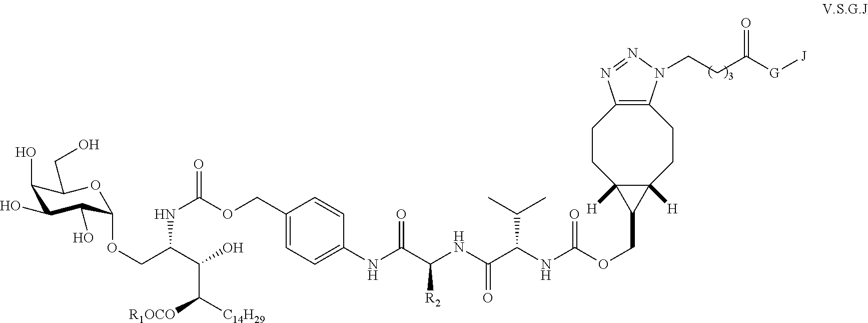

wherein
R₁ is (C₁₇-C₂₅)alkyl,
R₂ is the side-chain for alanine or citrulline,
G is absent or is an amino acid sequence selected from the group consisting of
FFRK (SEQ ID NO: 1), FKFL (SEQ ID NO: 16) and GFLG (SEQ ID NO: 17); and
J is a peptide antigen.

In one embodiment, the compound of Formula I is a compound of Formula V.Ox.G.J:

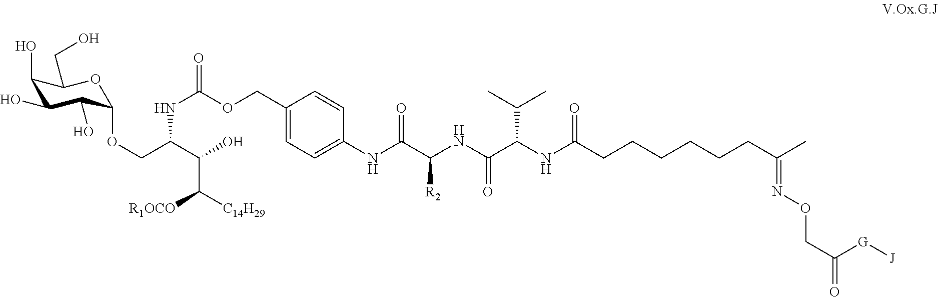

wherein
R₁ is (C₁₇-C₂₅)alkyl,
R₂ is the side-chain for alanine or citrulline,
G is absent or is the amino acid sequence FFRK (SEQ ID NO: 1), FKFL (SEQ ID NO: 16) or GFLG (SEQ ID NO: 17) and
J is a peptide antigen.

In one embodiment, J is a peptide antigen that is expressed by an organism that infects at least one cell in the liver of a subject.

In one embodiment, the peptide antigen is recognized by CD8⁺ T cells and increases the number of liver tissue resident memory T (T_{RM}) cells in response to the infective organism.

In one embodiment the peptide antigen is a Plasmodium spp. antigen, preferably a P. berghei antigen, preferably a P. berghei ANKA antigen.

In one embodiment R₁ is (C₁₉-C₂₅)alkyl, preferably (C₂₁-C₂₅)alkyl, more preferably (C₂₅)alkyl.

In one embodiment, G is FFRK (SEQ ID NO: 1).

In one embodiment, J is selected from the group consisting of NVYDFNLL (SEQ ID NO: 2) (NVY_{SP}), AAAHSLSNVYDFNLLLERD (SEQ ID NO: 3) (NVY_{LP}), NVFDFNNL (SEQ ID NO: 4) (NVF_{SP}) and AAASTNVFDFNNLS (SEQ ID NO: 5) (NVF_{LP}), DNQKDIYYITGESINAVS (SEQ ID NO: 6), AAALTSALLNVDNLIQ (SEQ ID NO: 7), STNVFDFNNLS (SEQ ID NO: 8), EIYIFTNI (SEQ ID NO: 13), ILNSGLLAV (SEQ ID NO: 18), TKILNSGLLAVVG (SEQ ID NO: 19), and HSLSILNSGLLAVLERD (SEQ ID NO: 20).

In one embodiment the compound of Formula I is selected from the group consisting of

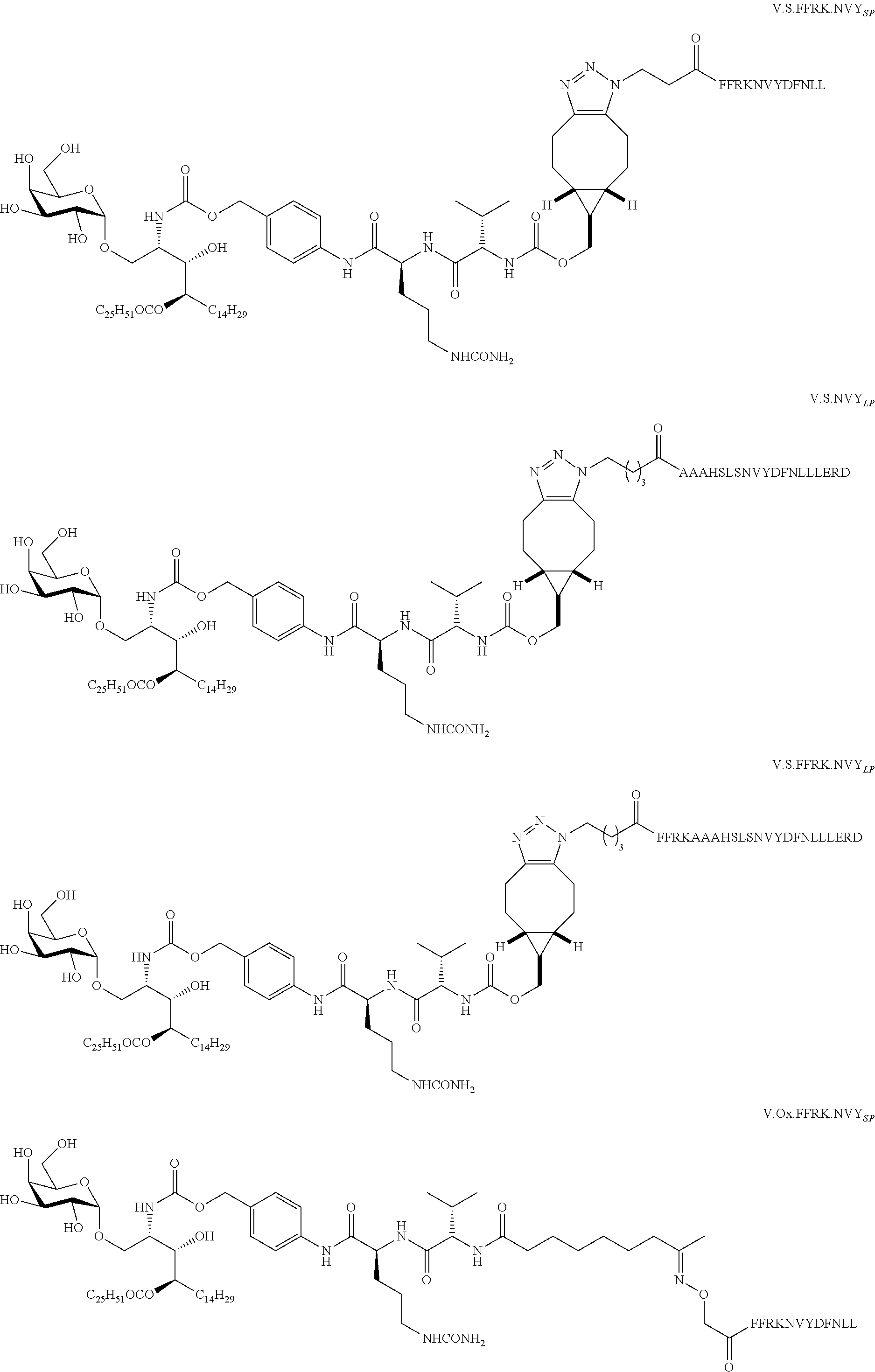

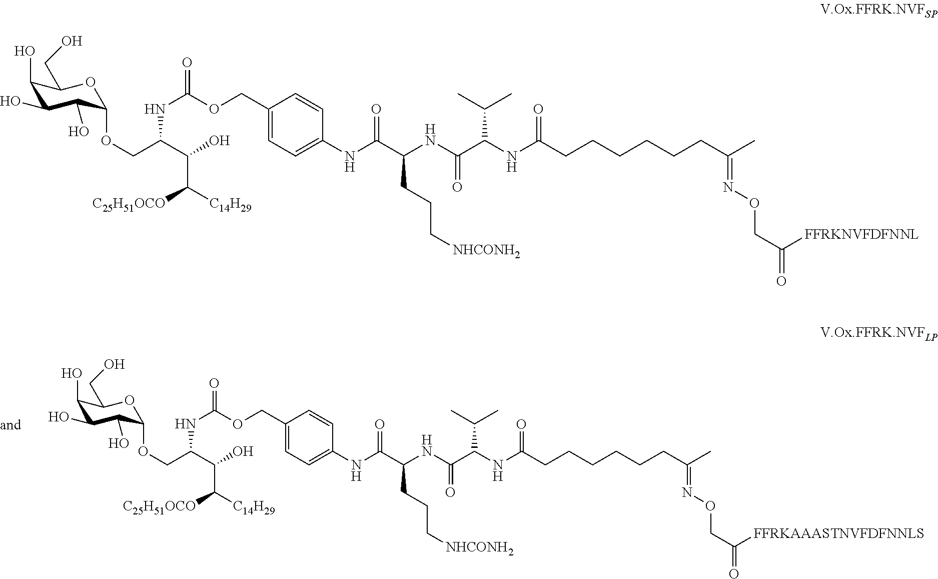

In one aspect the invention relates to a pharmaceutical composition comprising a compound of Formula I and at least one pharmaceutically acceptable carrier or excipient.

In one aspect the invention relates to a vaccine comprising a compound of Formula I and a pharmaceutically acceptable carrier or excipient.

In another aspect the invention relates to a method of increasing the number of liver T_{RM} cells in a subject comprising administering a compound of Formula I to the subject.

In another aspect the invention relates to a method of inducing an immune response that will reduce liver cell infection in a subject comprising administering a compound of Formula I to the subject.

In another aspect the invention relates to a method of vaccinating a subject against a hepatic infection comprising administering a compound of Formula I to the subject.

Various embodiments of the different aspects of the invention as discussed above are also set out below in the detailed description of the invention, but the invention is not limited thereto.

Other aspects of the invention may become apparent from the following description which is given by way of example only and with reference to the accompanying drawings.

### 4. BRIEF DESCRIPTION OF THE FIGURES

The invention will now be described by way of example only and with reference to the drawings in which:

FIG. 1 shows that few adjuvants enhance liver T_{RM} cell generation during priming using Clec9A-targeted antigen.

As described in Example 5, 50,000 PbT-I.GFP cells were transferred into B6 mice one day prior to vaccination with 2 μg of αClec9a-AAASTNVFDFNNLS (SEQ ID NO: 5) (containing the NVFDFNNL (SEQ ID NO: 4) PbT-I epitope) together with one of the following adjuvants:
i. 75 μg of CpG class B (ODN2006) linked to a CpG class P (21798),
ii. 50 μg of Poly I:C,
iii. 40 μg of RIG-I-ligand (5′ 3p dsRNA) complexed in in vivo Jet-PEI®,
iv. 75 μg of TLR7-ligand (ssRNA) complexed in DOTAP,
v. 75 μg of cGAS-ligand (dsDNA) complexed in in vivo Jet-PEI®
vi. 1 μg of LPS.

Livers (A) and spleens (B) were harvested from all mice 28 days later and assessed for the generation of memory T cells by flow cytometry. Memory cells were defined as CD8⁺GFP⁺CD44⁺; from this initial gating three memory subpopulations were determined as T_{RM} (CD69+CD62L^{low}), effector memory T cells (T_{EM})(CD69-CD62L^{low}) and central memory T cells (T_{CM})(CD69-CD62L^{high}) cells. Results are from 2 independent experiments using at least 3 mice per group for each experiment. Data displayed show mean±S.E.M.

FIG. 2 shows that infection with mouse cytomegalovirus (MCMV) expressing the malaria antigen TRAP does not induce high numbers of liver T_{RM} cells.

As described in Example 5, C57BL/6 mice were intravenously vaccinated by prime-and-trap using Clec9A-TRAP (SALLNVDN—SEQ ID NO: 9) with CpG and rAAV-TRAP or they were infected with 10⁵ PFU of recombinant MCMV expressing the malaria TRAP antigen (MCMV-TRAP). 36 days after vaccination spleens (A) and livers (B) were recovered and assessed by flow cytometry by staining with the H-2D^{b}-SALLNVDN tetramer and cell surface markers CD8, CD44. CD62L, CD69. After gating on CD8⁺, CD44^{high} cells, the number of TRAP-specific T_{RM} (CD69+CD62L^{low}), T_{EM} (CD69−CD62L^{low}) and T_{CM} (CD69-CD62L^{high}) cells were enumerated. Results are from one experiment using at 4 mice per group. Data displayed show mean±S.E.M.

FIG. 3 shows that α-GalCer as a substitute for CpG in Prime and Trap vaccination does not generate large numbers of liver T_{RM} cells.

As described in Example 5, 50,000 PbT-I.GFP cells were transferred into C57BL/6 mice one day prior to vaccination with 8 μg of αClec9a-NVY and either 5 nmol of CpG or 0.135 nmol of α-GalCer. The following day all mice were infected with 2.5×10⁹ copies of rAAV-NVY. Spleens (A) and livers (B) were harvested from all mice 35 days later and the number of PbT-I T_{RM} (CD69⁺CD62L^{low}), T_{EM} (CD69−CD62L^{low}) and T_{CM} (CD69−CD62L^{high}) cells were enumerated by flow cytometry. Data displayed are from one experiment using 5 mice per group.

FIG. 4 shows that the conjugate compounds of the invention provide an increase in the number of liver T_{RM} cells in mice.

As described in Example 6, 40,000 Ly5.1⁺OT-I cells were adoptively transferred into recipient C57BL/6 mice. 1 day later the recipient mice were treated with either PR8-OVA, α-GalCer and OVA long peptide [αGC+OVA_{LP}] or a conjugate vaccine [V.S.FFRK.OVA_{LP}] containing the OVA long peptide. Livers and spleens were harvested from recipient mice at days 21 and 60 post vaccination and assessed for the generation of memory T cells by flow cytometry (see FIG. 5).

FIG. 4A Phenotype of T_{RM} cells (CD8⁺Ly5.1⁺ CD44⁺ CD69⁺ CD62L^{low}) and T_{EM} cells (CD8⁺Ly5.1⁺ CD44⁺ CD69⁻CD62L^{low}) in the liver at day 21 after vaccination with V.S.FFRK.OVA_{LP}. FIG. 4B. Number of T_{RM} cells present in the liver at day 21 post vaccination. FIG. 4C. Number of T_{RM}, T_{EM}, and T_{CM} (CD8⁺Ly5.1⁺ CD44⁺ CD69⁻ CD62L^{high}) cells present in the liver at day 21 post vaccination. FIG. 4D. Number of T_{RM}, T_{EM}, and T_{CM} cells present in the spleen at day 21 post vaccination. FIG. 4E. Number of T_{RM} cells present in the liver at day 60 post vaccination. FIG. 4F. Number of T_{RM}, T_{EM}, and T_{CM} cells present in the liver at day 60 post vaccination. FIG. 4G. Number of T_{RM}, T_{EM}, and T_{CM} cells present in the spleen at day 60 post vaccination. Results in FIG. 4A-G are from two independent experiments using a total of 10 mice in the two experiments.

FIG. 5 shows the detection of memory CD8⁺ T cell populations.

As described in Example 7, liver and spleen lymphocytes were gated using FSC-A and SSC-A profiles. Doublets were removed from the analysis using FSC-A vs FSC-H and dead cells were removed by eliminating cells positive for propidium iodide staining. Donor CD8⁺ T cells populations were then selected by gating on CD45.1 or Ly5.1⁺ cells followed by cells expressing the activation/memory marker CD44 and the Vα2 transgenic TCR chain. Memory subsets were delineated using antibodies against CD69 and CD62L. For experiments using PbT-I DC8⁺ T cells, donor cells were selected using GFP.

FIG. 6 shows that conjugate compounds with longer fatty acid chains expand and activate splenic and liver NKT cells.

As described in Example 9, C57BL/6 mice were vaccinated i.v. with α-GalCer or conjugate vaccines V.S.FFRK.OVA_{LP} C26-C0 (0.135 nmol) to examine level of activation of NKT cells. Analysis was conducted by flow cytometry on liver and spleen 3 days after injection. Lymphocytes were gated on basis of FSC and SSC profiles, with doublets removed, and then dead cells were excluded by eliminating cells DAPI positive cells. Antibody to CD45R/B220 was used to exclude B cells. NKT cells were detected with fluorescent CD1d/α-GalCer tetramers and antibody to CD3. Activation status of NKT cells was determined by examining expression of NK1.1 and CD69, both of which are downregulated by day 3. Graphs show numbers of tetramer⁺CD3^{int} cells (NKT cells) in the spleen (A) and liver (D) at day 3, the percentage of NK1.1 positive NKT cells in spleen (B) and liver (E) at day 3, and the percentage of CD69 positive NKT cells in spleen (C) and liver (F) at day 3. Data from individual mice as well as mean±S.E.M. are presented.

FIG. 7 shows that conjugate compounds with long fatty acyl chains are optimal for liver T_{RM} cell formation and protection from malaria.

As described in Example 9, C57BL/6 mice were immunised with OVA conjugate vaccines (0.135 nmol) of varying fatty acyl chain length (C26-C0) one day after intravenous transfer of 5×10⁴ naïve CD45.1⁺ OT-I T cells. (A to C) Mice were sacrificed at day 21 post-immunisation, and spleens and livers were assessed for memory CD8⁺ T cell formation by FACS. The absolute numbers of CD44⁺ CD8⁺ memory OT-I subsets, [T_{CM} (CD69⁻ CD62L⁺), T_{EM} (CD69⁻ CD62L⁻) and T_{RM} (CD69⁺CD62L⁻) cells] in the liver (A, B) and spleen (C) were determined. Data are pooled from 2 independent experiments using a total of 7-8 mice/group. Error bars represent mean±S.E.M; one-way ANOVA with Tukey's multiple comparison. (D and E) The remaining cohorts of immunised mice as described in (A-C) and unvaccinated mice were challenged with 200 OVA transgenic PbA sporozoites at day 28 post-immunisation and parasitemia was measured up to 12 after challenge. (D) Parasitemia (% infected RBCs) at day 7 post-challenge. Means±S.E.M are shown. (E) The level of sterile protection, as determined by the absence of blood-stage parasitemia at day 12 after challenge.

Data are pooled from 2 independent experiments using a total of 11 mice/group. Groups were compared using one-way ANOVA with Tukey's multiple comparison test in (D) and Fisher's exact test in (E).

FIG. 8 shows that vaccination with the conjugate compound V.S.FFRK.NVY_{SP} protects a portion of mice from malaria.

As described in Example 10, 50,000 PbT-I.GFP cells were transferred into recipient C57BL/6 mice. After one day, the recipient mice were treated with αClec9a-NVY/CpG, α-GalCer alone (αGC), or a conjugate vaccine containing NVY short peptide [V.S.FFRK.NVY_{SP}]. Mice treated with αClec9a-NVY/CpG were also treated with rAAV-NVY at day 1 (P&T). Organs were harvested from mice from each group at day 35 post vaccination and assessed for the generation of memory T cells by flow cytometry. FIG. 8A. Number of T_{RM} cells (CD8⁺GFP⁺ CD44⁺ CD69⁺ CD62L^{low} KLRG1⁻) present in the liver at day 35 post vaccination. FIG. 8B. Phenotype of T_{RM} and T_{EM} cells in the liver 35 days after vaccination with V.S.FFRK.NVY_{SP}. FIG. 8C and FIG. 8D. The number of PbT-I T_{RM}, T_{EM} (CD44⁺ CD69⁻ CD62L^{low}), and T_{CM} (CD44⁺ CD69⁻ CD62L^{high}) cells in the liver (C) and spleen (D) at day 35 post vaccination.

The remaining mice were challenged with 200 P. berghei sporozoites at day 42 and parasitemia was measured by flow cytometry from day 6-13. FIG. 8E. Percentage of parasites present in red blood cells at day 7 post malaria challenge. FIG. 8F. Number of mice that succumbed or were protected after malaria challenge. FIG. 8G. Depletion of liver T^{RM} cells with anti-CXCR3 mAb abrogates protection.

Results are from 2 or 3 independent experiments using at least 4 mice per group for each experiment, with the exception of the naïve group. Data displayed show mean±S.E.M and in some cases (A, E) data from individual mice. Groups in A and E were compared by one way ANOVA with Tukey's multiple comparison post-test. Groups in F were compared using Fisher's exact test. **** p<0.001.

FIG. 9 shows that the conjugate compound V.S.FFRK.NVY_{SP} is an effective prime-boost vaccine.

As described in Example 8, 50,000 PbT-I.GFP cells were transferred into recipient C57BL/6 or CD1d^{-/-} mice. CD1d^{-/-} mice were treated with V.S.FFRK.NVY_{SP} at day 0 and 30 (Group 1). C57BL/6 mice were treated with V.S.FFRK.NVY_{SP} at day 30 only (Group 2), V.S.FFRK.NVY_{SP} at day 0 and 30 (Group 3), αClec9a-NVY and CpG at day 0 and V.S.FFRK.NVY_{SP} at day 30 (group 4), or with αClec9a-NVY and CpG at day 0 (Group 5). Organs were harvested from mice from each group at day 50-60 and assessed for the generation of memory T cells. FIG. 9A, number of liver PbT-I T_{RM} cells at day 50-60 post vaccination. FIG. 9B and FIG. 9C, number of T_{RM}, T_{EM} and T_{CM} cells present in the liver (B) and spleen (C) at day 50-60 post vaccination.

The remaining mice in each group were challenged with 200 P. berghei sporozoites at day 73 and parasitemia was measured at day 79, 80, 81. Mice with two consecutive days of visible parasites in the blood were culled. Mice surviving challenge with low dose sporozoites were rechallenged with 3000 sporozoites. Parasitemia was measured at days 5, 6, 7, 8 and 12 post-high-dose-challenge. FIG. 9D, percentage of parasites present in red blood cells at day 7 post primary malaria challenge. This is 80 days after the start of the experiment. FIG. 9E, number of mice that succumbed or were protected after 200, or 200 and 3000 sporozoite challenge.

FIG. 10 shows that the conjugate compounds described herein containing peptide flanking residues generate large numbers of liver T_{RM} cells.

As described in Example 11, 50,000 PbT-I.GFP cells were transferred into recipient C57BL/6 mice. After one day, the recipient mice were treated with α-GalCer alone (αGC), α-GalCer and NVY long peptide (αGC+NVY_{LP}), short peptide (V.S.FFRK.NVY_{SP}) and long peptide (V.S.FFRK.NVY_{LP}) conjugate vaccines, or a long peptide conjugate vaccine lacking the FFRK cleavage sequence (V.S.NVY_{LP}). Organs were harvested from mice from each group at days 21-35 post vaccination and assessed for the generation of memory T cells by flow cytometry as outlined in FIG. 5. The remaining mice per group were challenged with 200 P. berghei sporozoites at day 42 and parasitemia was measured by flow cytometry at days 6, 7, 8 and 13. Mice with two consecutive days of visible parasites in the blood were culled. FIG. 10A, number of liver T_{RM} cells at days 21-35 post vaccination. FIGS. 10B and C, number of T_{RM}, T_{EM}, and T_{CM} cells present in the liver (B) and spleen (C) at days 21-35 post vaccination. FIG. 10D, percentage of parasites present in red blood cells at day 7 post-malaria challenge. FIG. 10E, number of mice that succumbed or were protected after 200 sporozoite challenge.

FIG. 11 shows that conjugate compound V.S.NVY_{LP} does not expand or activate splenic or liver NKT cells.

As described in Example 11, C57BL/6 mice were vaccinated with α-GalCer or conjugate vaccines V.S.FFRK.NYY_{SP}, V.S.FFRK.NVY_{LP} or V.S.NVY_{LP}. Figs A-B, CD1d tetramer positive NKT cell numbers in the liver (A) and spleen (B) at day 3. Figs C-D, mean fluorescence intensity of CD69 on NKT cells in the liver (D) and spleen (E) at day 3. Figs E-F, percentage of NKT cells in the liver (E) and spleen (F) expressing NK1.1 at day 3. Fig G, serum ALT was measured 18 hrs post vaccination. The graphs displays data from individual mice as well as mean±SEM. Groups were compared by one way ANOVA with Tukey's multiple comparison post-test. *p<0.05.

FIG. 12 shows that the conjugate compounds linked as described herein using oxime chemistry (V.Ox.G.J compounds) elicit sterile immunity against Plasmodium.

As described in Example 12, 50,000 PbT-I.GFP cells were transferred into recipient C57BL/6 mice. After one day, the recipient mice were treated with V.Ox.FFRK.NVY_{SP} or V.S.FFRK.NVY_{SP} conjugates. Organs were harvested from mice from each group at days 21 post vaccination and assessed for the generation of memory T cells by flow cytometry as outlined in FIG. 5. The remaining mice per group were challenged with 200 P. berghei sporozoites at day 35 and parasitemia was measured by flow cytometry at days 6, 7, 8 and 13. Mice with two consecutive days of visible parasites in the blood were culled. FIG. 12A, number of liver T_{RM} cells at days 21 post vaccination. FIG. 12B and FIG. 12C, number of T_{RM}, T_{EM}, and T_{CM} cells present in the liver (B) and spleen (C) at day 35 post vaccination. FIG. 12D, percentage of parasites present in red blood cells at day 7 post 200 sporozoite challenge. FIG. 12E, number of mice that succumbed or were protected after malaria challenge. FIG. 12F, percentage of parasites present in red blood cells at day 7 post 3000 sporozoite challenge. FIG. 12G, number of mice that succumbed or were protected after malaria challenge.

FIG. 13 shows that the conjugate compounds can generate endogenous CD8⁺ memory T cells specific to a malaria epitope.

As discussed in Example 13, C57BL/6 mice were injected with 0.135 nmoles of a glycolipid peptide conjugate vaccine including NVFDFNNL (SEQ ID NO: 4) (V.Ox.FFRK.NVF_{SP}) at days 0, 14 and 35 (prime-boost-boost, PBB) or were left unprimed (naïve). Tetramer⁺ memory CD8⁺ T cells in the spleen and the liver of the PBB group were enumerated 56 days after immunization (A). Data were pooled from 2 independent experiments, with a total of 4 mice/group. (B) Naïve mice and PBB mice vaccinated 3 times with V.Ox.FFRK.NVF_{SP} were challenged with 200 WT PbA sporozoites 57 days after vaccination. Protection was considered sterile when blood stage parasites had not been detected up to day 12 after challenge. Data were pooled from 2 independent experiments, with a total of up to 12 mice/group.

FIG. 14 shows that a single dose of the conjugate compound can protect mice from malaria.

As discussed in Example 14, C57BL/6 mice were injected with 0.135 nmoles of a glycolipid peptide conjugate vaccine (V.Ox.FFRK.NVF_{LP}) including NVFDFNNL (SEQ ID NO: 4) with flanking sequences [AAASTNVFDFNNLS (SEQ ID NO: 5)] at day 0. Tetramer⁺ memory CD8⁺ T cells in the spleen and the liver were enumerated 35 days after immunization (A). Data were pooled from 2 independent experiments, with a total of 10 mice/group. Naïve mice and vaccinated mice were challenged with 200 WT PbA sporozoites 42 days after vaccination (B). Protection was considered sterile when blood stage parasites had not been detected up to day 12 after challenge. Surviving mice were rechallenged with 3000 sporozoites on day 70 post-vaccination. Parasitemia was measured at days 5, 6, 7, 8 and 12 post-high-dose-challenge. Data were pooled from 2 independent experiments, with a total of 10 mice/group.

FIG. 15 shows that a second dose of the conjugate compound can enhance protection malaria.

As discussed in Example 15, C57BL/6 mice were injected with 0.135 nmoles of a glycolipid peptide conjugate vaccine (V.Ox.FFRK.NVF_{LP}) including NVFDFNNL (SEQ ID NO: 2) with flanking sequences [AAASTNVFDFNNLS (SEQ ID NO: 5)] at day 0 only (Group 1—NVF/-), V.Ox.FFRK.NVF_{LP} at day 30 only (Group 2—-/NVF) or V.Ox.FFRK.NVF_{LP} at days 0 and 30 (Group 3—NVF/NVF). Tetramer⁺ memory CD8⁺ T cells in the liver (A) and spleen (B) were enumerated at day 60 relative to the day 0 immunization. Data were pooled from 2 independent experiments, with a total of 10 mice/group. Naïve mice and vaccinated mice were challenged with 200 WT PbA sporozoites 66 days after the day 0 vaccination (C). Protection was considered sterile when blood stage parasites had not been detected up to day 12 after challenge. Surviving mice were rechallenged with 3000 sporozoites on day 85 post day 0 vaccination. Parasitemia was measured at days 5, 6, 7, 8 and 12 post-high-dose-challenge. Data were pooled from 2 independent experiments, with a total of 10-11 mice/group.

FIG. 16 shows that protection from a single dose is maintained for 200 days.

As discussed in Example 16, C57BL/6 mice were injected with 0.135 nmoles of a glycolipid peptide conjugate vaccine (V.Ox.FFRK.NVF_{LP}) at day 0. Tetramer⁺ memory CD8⁺ T cells in the liver were enumerated at various time points over 200 days (A). Data were pooled from 2-3 independent experiments, with a total of >9 mice/group. Additional mice were challenged with 200 P. berghei sporozoites at the time of harvest. (B) Protection was considered sterile when blood stage parasites had not been detected up to day 12 after challenge. Data were pooled from 2 independent experiments (with the exception of day 35 and day 75 which are from a single experiment), with a total of 10-11 mice/group.

FIG. 17 shows that protection from a single dose is superior to the current gold-standard malaria vaccine, radiation attenuated sporozoites (RAS).

As discussed in Example 17, C57BL/6 mice were injected with 0.135 nmoles of a glycolipid peptide conjugate vaccine (V.Ox.FFRK.NVF_{LP}) or 50,000 irradiated sporozoites (RAS) and challenged one month later with 200 P. berghei sporozoites. Livers were harvested at the time of euthanasia, which was either upon detection of blood-stage infection (day 7 post-challenge—grey circles) or once mice had been assessed as protected (day 12—open circles). Liver cells were then assessed for the presence of NVF-specific T_{RM} cells (A) or total liver T_{RM} cells (B). Protection data is outlined in (C). Data were pooled from 2 independent experiments with a total of 10-11 mice/group.

FIG. 18 shows identification of the HLA-A*02:01-restricted epitope ILNSGLLAV (SEQ ID NO: 18) in P. falciparum RPL6 (PfRPL6 (PF3D7_1338200)).

HHD mice, which express human HLA-A*02:01 and lack expression of murine MHC class molecules, were immunized with 25 mg anti-CD40 mAb+25 mg poly IC+100 mg of PfRPL6₇₇₋₈₅ peptide (ILNSGLLAV) (SEQ ID NO: 18), or no peptide. 7 days later, PfRPL6₇₇₋₈₅-specific responses were measured in the spleen by ELISpot, by restimulating with the same peptide. Data were analysed using two-way ANOVA; Number of experiments=2, number of mice=3-4.

### 5. DETAILED DESCRIPTION OF THE INVENTION

### 5.1 Definitions

The following definitions are presented to better define the present invention and as a guide for those of ordinary skill in the art in the practice of the present invention.

Unless otherwise specified, all technical and scientific terms used herein are to be understood as having the same meanings as is understood by one of ordinary skill in the relevant art to which this disclosure pertains. It is also believed that practice of the present invention can be performed using standard microbiological, molecular biology, pharmacology and biochemistry protocols and procedures as known in the art.

The terms “administering” or “administration” refer to placement of a compound of the invention into a subject by a method appropriate to result in an immune response.

As used herein the term “increase” (and grammatical variations thereof) as used herein with reference to liver tissue resident memory CD8⁺ T cells (T_{RM}) means a measurable or observable increase in the number of liver T_{RM} T cells in a subject treated with a conjugate compound as described herein relative to the number of liver T_{RM} cells observed in an appropriate control (e.g., untreated) subject; e.g., placebo or non-active agent. In preferred embodiments the measurable or detectable increase is a statistically significant increase, relative to an appropriate control.

A “therapeutically effective amount” (or “effective amount”) is an amount sufficient to effect beneficial or desired results, including clinical results, but not limited thereto. A therapeutically effective amount can be administered in one or more administrations by various routes of administration. The therapeutically effective amount of the conjugate compound to be administered to a subject depends on, for example, the purpose for which the compound is administered, mode of administration, nature and dosage of any co-administered compounds, and characteristics of the subject, such as general health, other diseases, age, sex, genotype, body weight and tolerance to drugs. A person skilled in the art will be able to determine appropriate dosages having regard to these any other relevant factors.

In the context of the present disclosure, a therapeutically effective amount of the compound that is useful as a human vaccine is the amount of the conjugate compound that is expected to be effective in a human based on the mouse data disclosed herein. Such an amount can be determined by the skilled worker using an appropriate conversion model.

A “subject” refers to a human or a non-human animal, preferably a vertebrate that is a mammal. Non-human mammals include, but are not limited to; livestock, such as, cattle, sheep, swine, deer, and goats; sport and companion animals, such as, dogs, cats, and horses; and research animals, such as, mice, rats, rabbits, and guinea pigs. Preferably, the subject is a human.

A “control subject” as used herein means a suitable control subject as would be recognized by a person of skill in the art to which the relevant experiment or assay pertained.

As used herein the term “prevents” and grammatical variations thereof when used in reference to a hepatic infection, particularly a parasite or pathogen infection, preferably a Plasmodium spp. infection, means that at least 10%, preferably at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, preferably all of the subjects treated with a compound of the invention will not be infected upon challenge with an agent that infects hepatocytes. “Sterile protection” means 100% of the subjects treated with a compound of the invention will not be infected upon challenge with an agent that infects hepatocytes.

The term “reduce” (and grammatical variations thereof) as used herein with reference to liver cell infection means a measurable or observable reduction in the number of infected cells in a subject, preferably the number of liver cells in a subject treated with a conjugate compound as described herein, relative to the number of infected cells observed in an appropriate control (e.g., untreated) subject; e.g., placebo or non-active agent. In preferred embodiments the measurable or detectable reduction is a statistically significant reduction, relative to an appropriate control.

The term, “an agent that infects hepatocytes” as used herein refers to any infectious agent or organism (e.g., fungal, bacterial, protist or viral) that can enter an animal body, particularly a human body, and infect liver cells. In some embodiments the agent is Plasmodium.

The term “vaccine” and grammatical variations as used herein refers to a substance that stimulates an immune response, i.e., that induces the production of antibodies that provide immunity against disease. A vaccine may be made from a disease agent, a product or part of a disease agent, or a synthetic substitute and acts to provoke an antigenic response without inducing the actual disease.

As used herein, a vaccine for hepatic infection, particularly a vaccine for a parasite infection, preferably a Plasmodium infection, refers to a substance that, upon administration to a subject, provides immunity from the infection in at least 10%, preferably at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, preferably all of the vaccinated subjects. Preferably the vaccine provides sterile protection to the vaccinated subjects.

The term “antigen” refers to a molecule that contains one or more epitopes (linear, overlapping, conformational or a combination of these) that, upon exposure to a subject, can induce an immune response that is specific for that antigen.

The term “peptide antigen” as used herein means an antigen that is expressed by an organism that infects at least one cell in the liver of a subject. In one embodiment, the peptide antigen is recognized by CD8⁺ T cells and increases the number of liver T_{RM} cells in response to the infective organism.

In some embodiments the peptide antigen increases the number of liver T_{RM} cells by at least 10, preferably by at least 1×10², preferably by at least 1×10³, preferably by at least 1×10⁴ cells in a subject that has been administered a compound as described herein, as compared to a suitable control subject as known in the art that has not been administered the compound.

In some embodiments the peptide antigen increases the number of liver T_{RM} cells by at least 10, preferably by at least 1×10², preferably by at least 1×10³, preferably by at least 1×10⁴, preferably by at least 1×10⁵, preferably by at least 1×10⁶ cells in a subject that has been administered a second dose of the compound as described herein as compared to a suitable control subject that has been administered only a single dose of the compound.

In some embodiments increasing the number of the liver T_{RM} cells comprises at least a two fold increase in the number of liver T_{RM} cells, preferably at least a five-fold increase, at least a 10 fold increase, at least a 100 fold increase, at least a 1000 fold increase, preferably at least a 10,000 fold increase in the number of liver T_{RM} cells in a subject that has been administered a compound as described herein, as compared to a suitable control subject as known in the art that has not been administered the compound.

In some embodiments increasing the number of the liver T_{RM} cells comprises at least a two fold increase in the number of liver T_{RM} cells, preferably at least a five-fold increase, at least a 10 fold increase, at least a 100 fold increase, at least a 1000 fold increase, at least a 10,000 fold increase, at least a 100,000 fold increase, preferably at least a 1,000,000 fold increase in the number of liver T_{RM} cells in a subject that has been administered a compound as described herein, as compared to a suitable control subject that has been administered only a single dose of the compound.

The term “antigenic challenge” as used herein means the exposure of at least one liver cell to an antigen that is expressed by an organism that infects at least one cell in the liver of a subject.

The term “pharmaceutically acceptable carrier or excipient” means a excipient or carrier that is compatible with the other ingredients of the composition, and not harmful to the subject to whom the composition is administered.

Numerous pharmaceutically acceptable carriers and excipients are approved by relevant government regulatory agencies. Examples of pharmaceutically acceptable carriers and excipients include sterile liquids such as water and oils, including animal, vegetable, synthetic or petroleum oils, saline solutions, aqeuous dextrose and glycerol solutions, starch glucose, lactose, sucrose, gelatin, sodium stearate, glycerol monostearate, sodium chloride, propylene glycol, ethanol, wetting agents, emulsifying agents, binders, dispersants, thickeners, lubricants, pH adjusters, solubilizers, softening agents, surfactants and the like. The compounds of the invention can be formulated in or as solutions, suspensions, emulsions, tablets, pills, capsules, powders and sustained-release formulations. Examples of suitable pharmaceutically acceptable carriers and excipients are described in Remington's Pharmaceutical Sciences 18th Ed., Gennaro, ed. (Mack Publishing Co. 1990). The presence of a pharmaceutically acceptable carrier or excipient in composition with a compound of the invention does not impair the activity of the compound of the invention.

The term “alkyl” means any saturated hydrocarbon radical having up to 30 carbon atoms and includes any C1-C25, C1-C20, C1-C15, C1-C10, or C1-C6 alkyl groups. In some embodiments, “alkyl” means any straight-chain saturated hydrocarbon radical having up to 30 carbon atoms.

The term “amino acid” includes both natural and non-natural amino acids.

The term “amide” includes both N-linked (—NHC(O)R) and C-linked (—C(O)NHR) amides.

For the purposes of the invention, any reference to the disclosed compounds includes all possible formulations, configurations, and conformations, for example, in free form (e.g. as a free acid or base), in the form of salts or hydrates, in the form of isomers (e.g. cis/trans isomers), stereoisomers such as enantiomers, diastereomers and epimers, in the form of mixtures of enantiomers or diastereomers, in the form of racemates or racemic mixtures, or in the form of individual enantiomers or diastereomers. Specific forms of the compounds are described in detail herein.

The term “about” when used in connection with a referenced numeric indication means the referenced numeric indication plus or minus up to 10% of that referenced numeric indication. For example, “about 100” means from 90 to 110 and “about six” means from 5.4 to 6.6.

The term “comprising” as used in this specification means “consisting at least in part of”. When interpreting statements in this specification that include that term, the features, prefaced by that term in each statement, all need to be present but other features can also be present. Related terms such as “comprise” and “comprised” are to be interpreted in the same manner.

The term “consisting essentially of” as used herein means the specified materials or steps and those that do not materially affect the basic and novel characteristic(s) of the claimed invention.

The term “consisting of” as used herein means the specified materials or steps of the claimed invention, excluding any element, step, or ingredient not specified in the claim.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

### 5.2 Detailed Description

The search for effective malaria vaccines has been hampered by a lack of understanding of how mammalian immune systems respond to the malaria-causing parasite Plasmodium spp.

30 years ago, vaccination with radiation attenuated sporozoites (RAS) was demonstrated to elicit sterile protection by targeting the pre-erythrocytic stage of the parasite life cycle. Protection against liver-stage infection was found to be mediated primarily by CD8⁺ T cells (Weiss W R, 1988) (Seguin M C, 1994) (Rodrigues M, 1993), with large numbers of malaria-specific CD8⁺ T cells required (Schmidt N W P. R., 2008) (Schmidt N W B. N., 2010).

However, while the importance of CD8⁺ T cell responses in malaria vaccination has long been known, the role of tissue-resident memory CD8⁺ T (T_{RM}) cells was only recently discovered. Unlike most memory T cells, which recirculate around the body, T_{RM} cells are a population of non-circulating memory T cells that reside permanently in tissues, acting as guards against pathogens. T_{RM} cells have a unique phenotype and can be distinguished from other memory T-cell types by expression of particular markers.

It was recently found that the generation of a large population of malaria-specific T_{RM} cells in the liver (using a prime-and-trap vaccination approach) was capable of eliciting sterile immunity (i.e. completely prevented egress of parasites from the liver to the blood) and was a more effective vaccination approach than the current gold standard pre-erythrocytic malaria vaccine, RAS. Protection by either vaccination method was absolutely dependent on the presence of T_{RM}, as depletion of this subset using anti-CXCR3 antibodies abrogated protection (Fernandez-Ruiz D, 2016).

Therefore, new vaccines that can stimulate production of liver T_{RM} cell populations may provide strong protection against malaria and other hepatic infections. Unfortunately, the factors that determine whether a vaccine will induce T_{RM} versus circulating T cells are poorly defined, making it difficult to predict which immunogenic agents might be capable of generating the large number of liver T_{RM} cells needed to provide an effective vaccine.

Studies have shown that local antigen expression and inflammation are key drivers of T_{RM} generation in the liver though (Fernandez-Ruiz D, 2016) (Holz L E, 2018) (Davies B, 2017) (Khan T N, 2016) (Mackay L K, 2012). While liver T_{RM} cells have been shown to develop in the absence of liver-associated antigen or inflammation, their numbers were significantly reduced (Holz L E, 2018). Therefore, it was theorized by the inventors that agents providing both inflammatory and antigenic signals in the liver might favor liver T_{RM} generation.

Adjuvants such as Toll-like receptor (TLR) agonists are an effective means of generating inflammation during vaccination and are useful for overcoming the poor immunostimulatory capacity of peptide vaccines, triggering enhanced T (16) and B cell responses. Therefore, the inventors investigated the possibility that such adjuvants might help malaria antigens boost liver T_{RM} cell populations, when administered together.

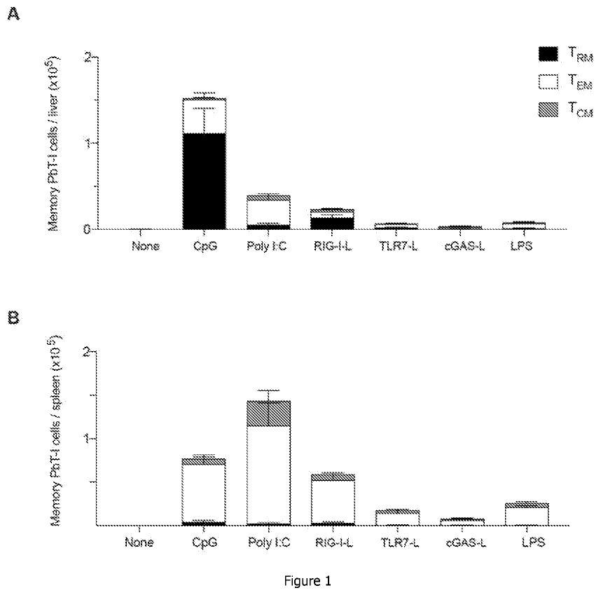

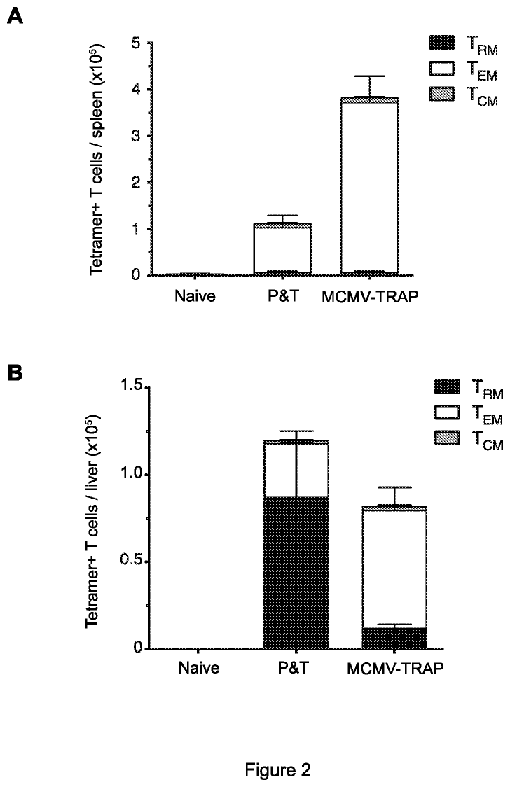

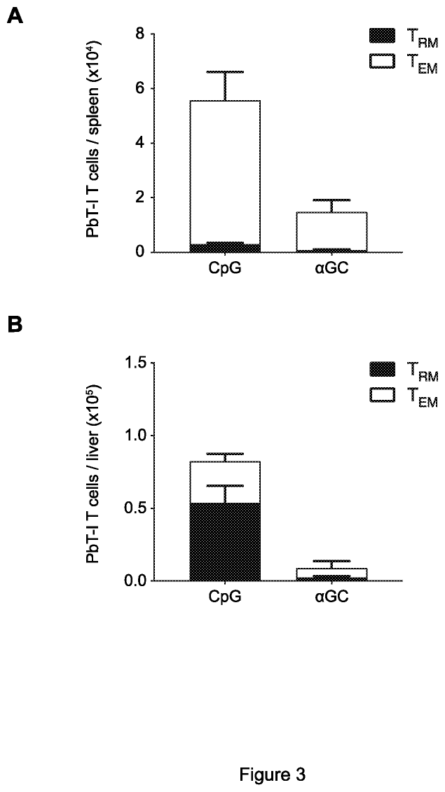

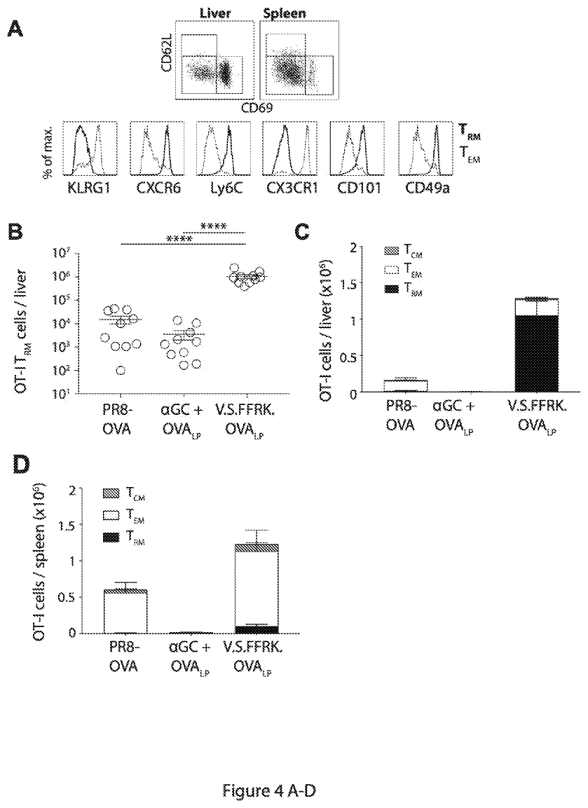

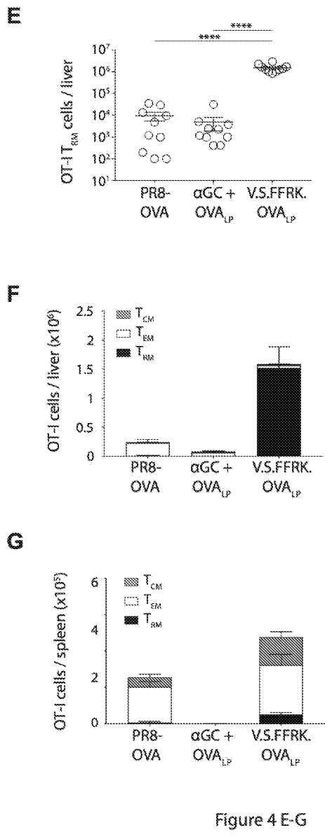

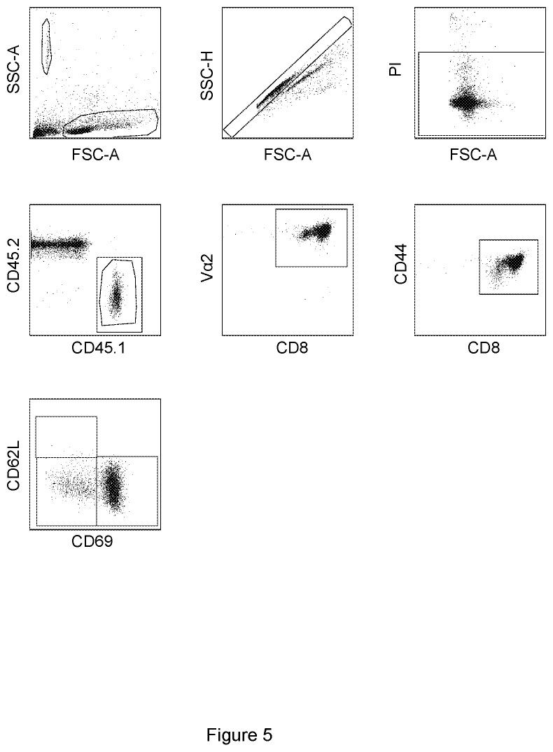

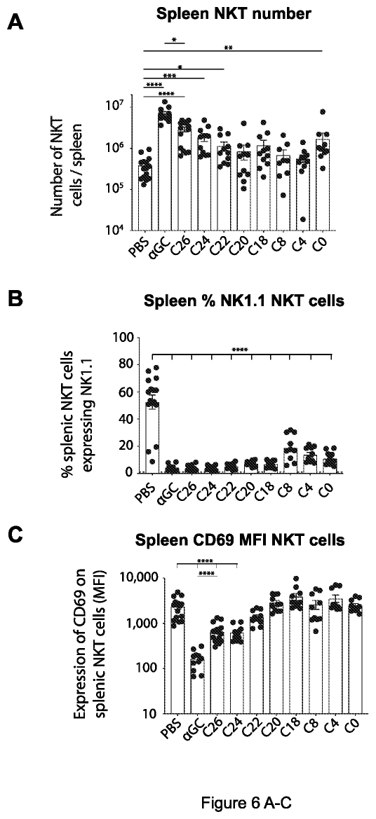

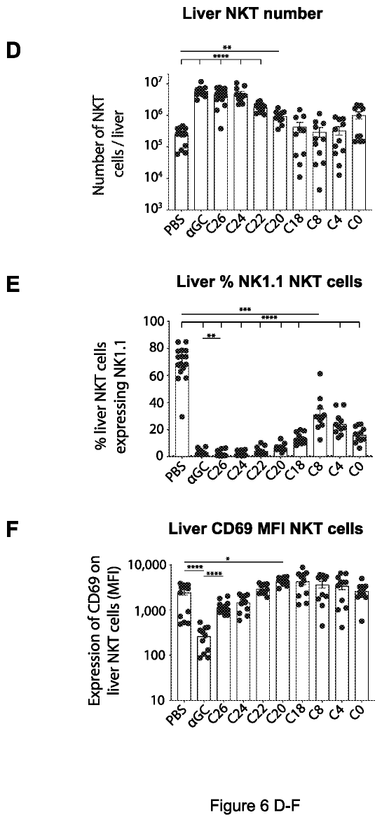

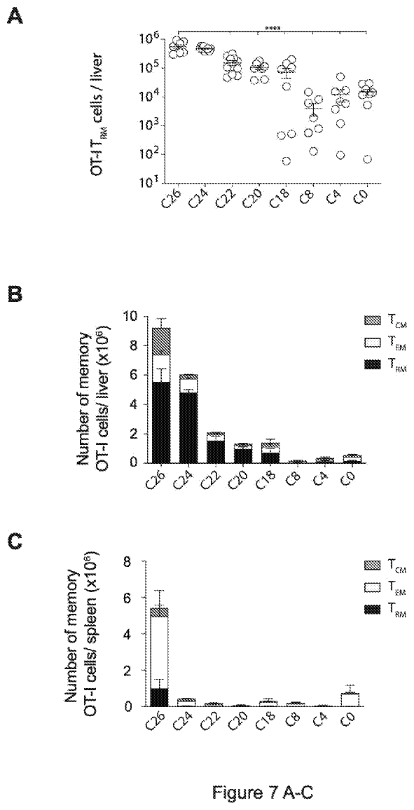

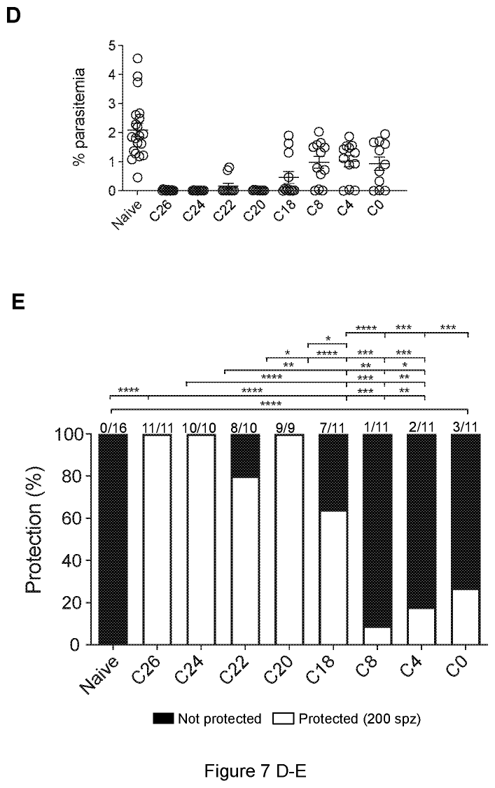

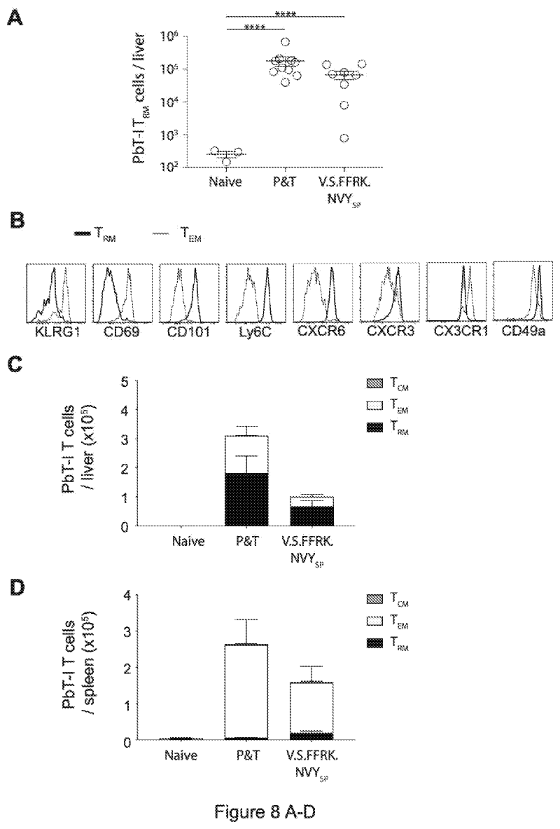

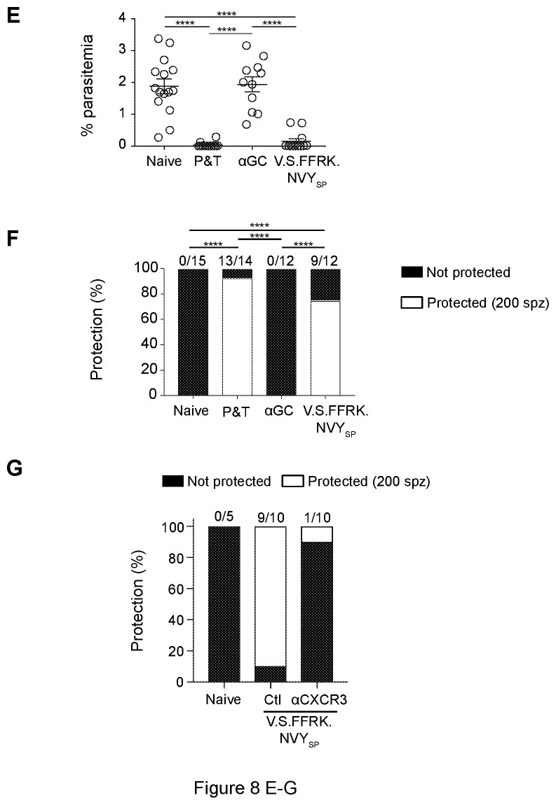

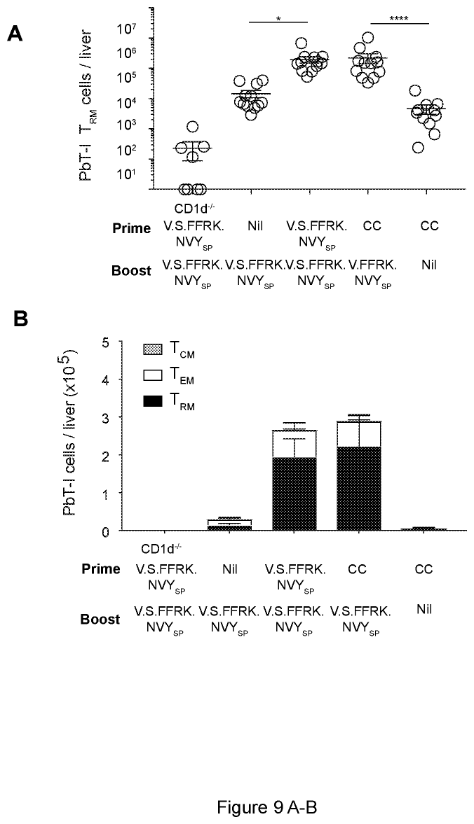

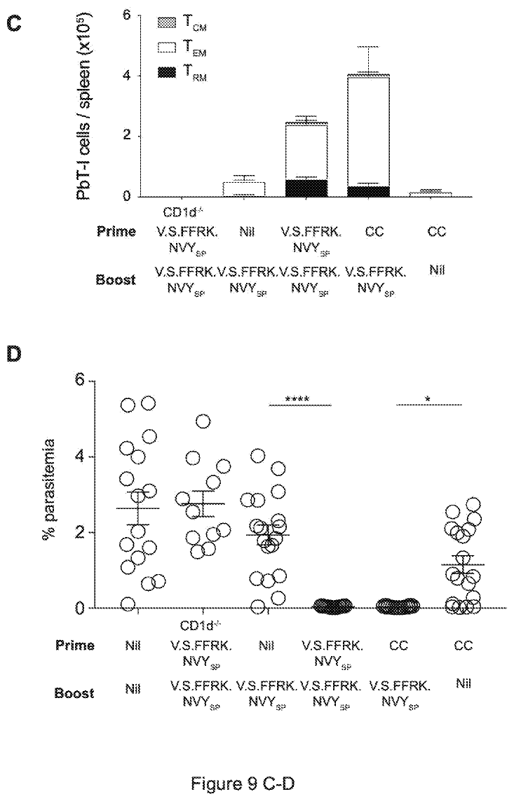

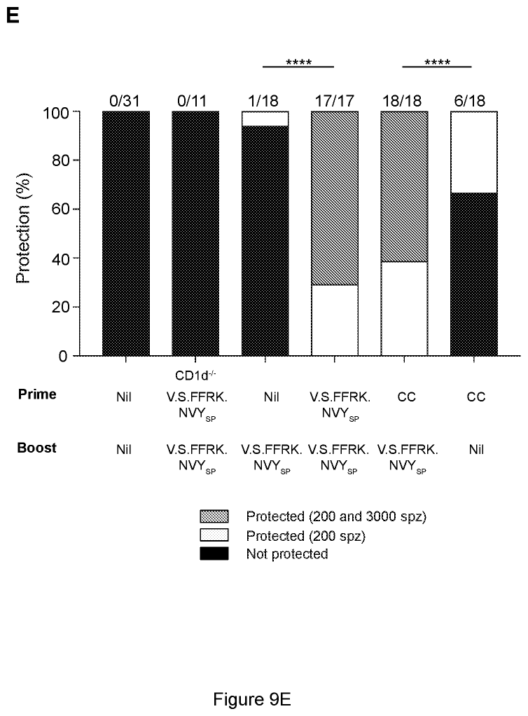

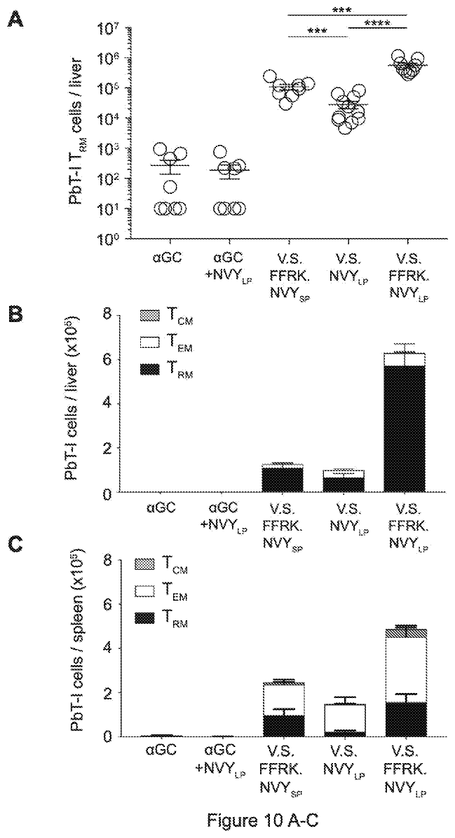

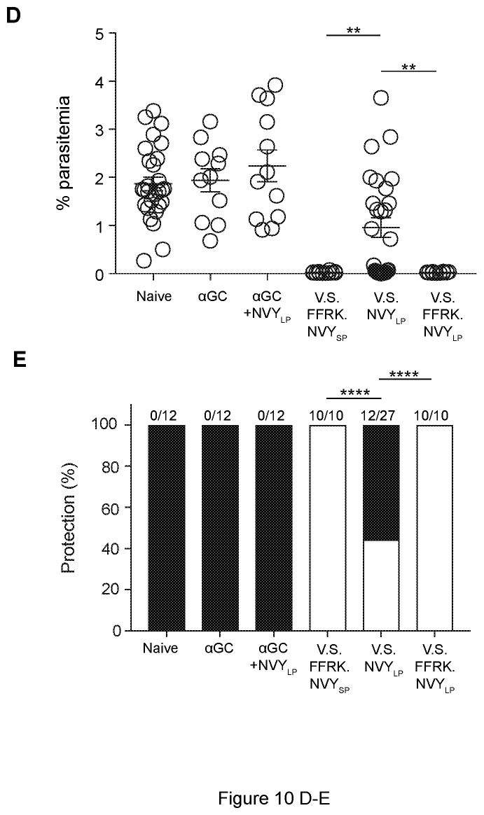

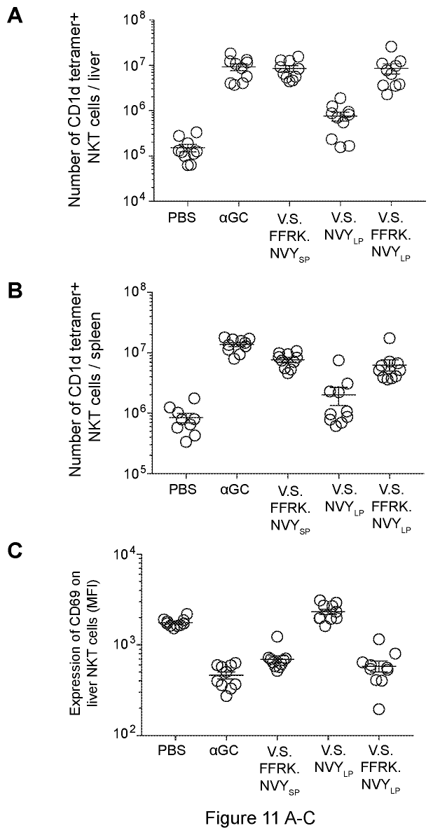

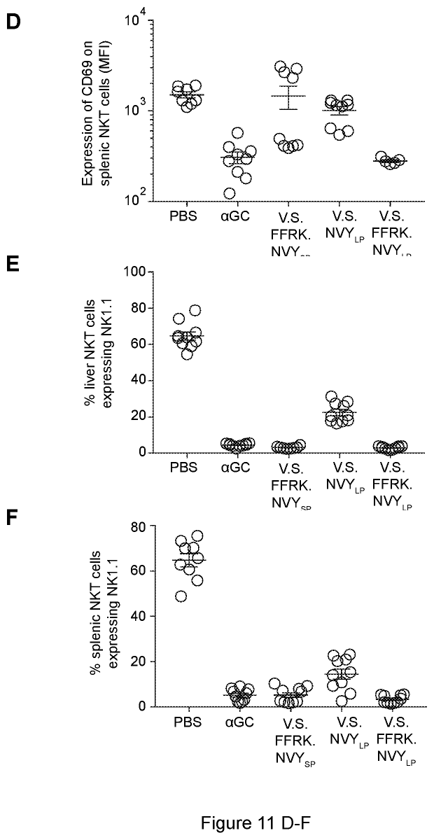

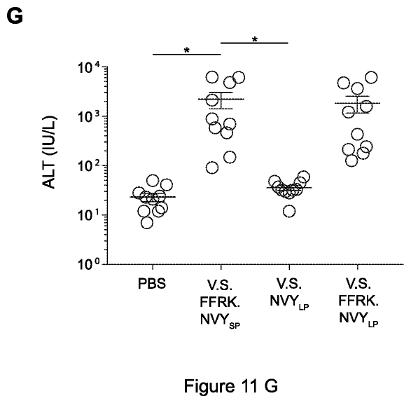

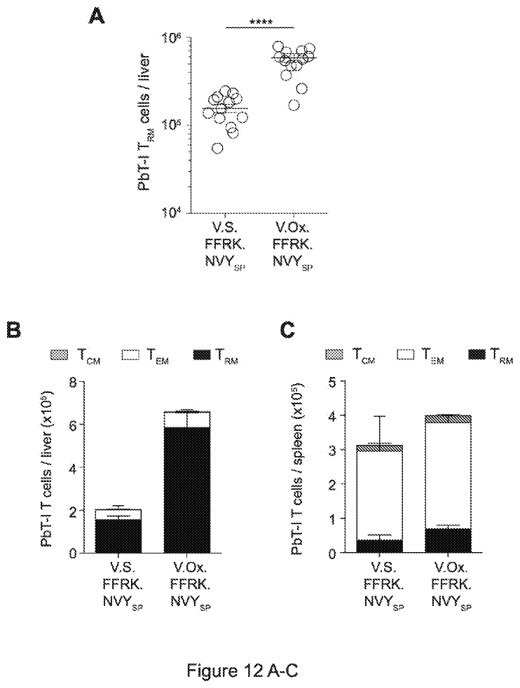

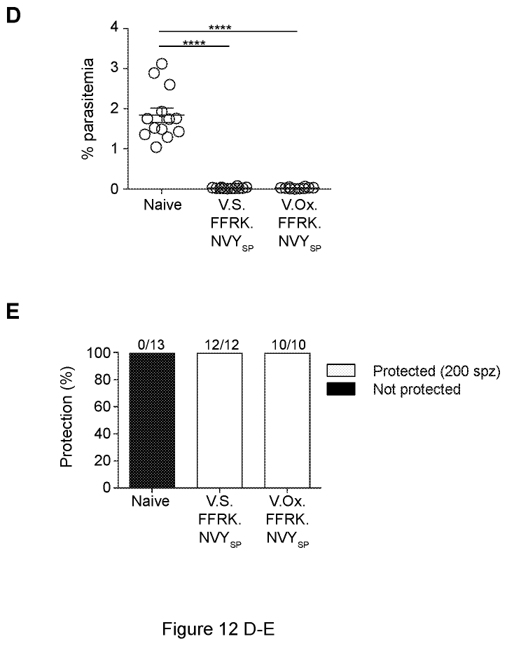

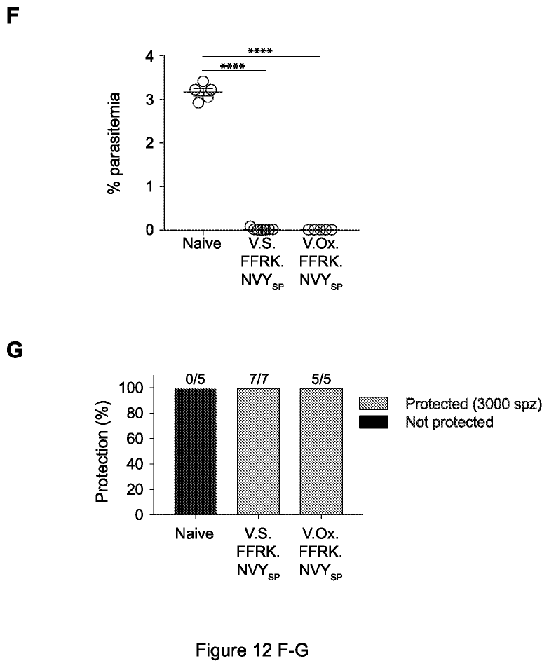

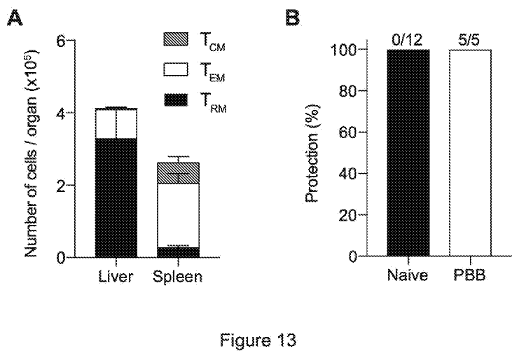

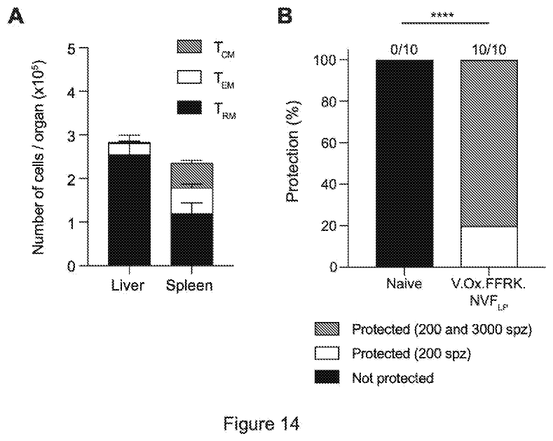

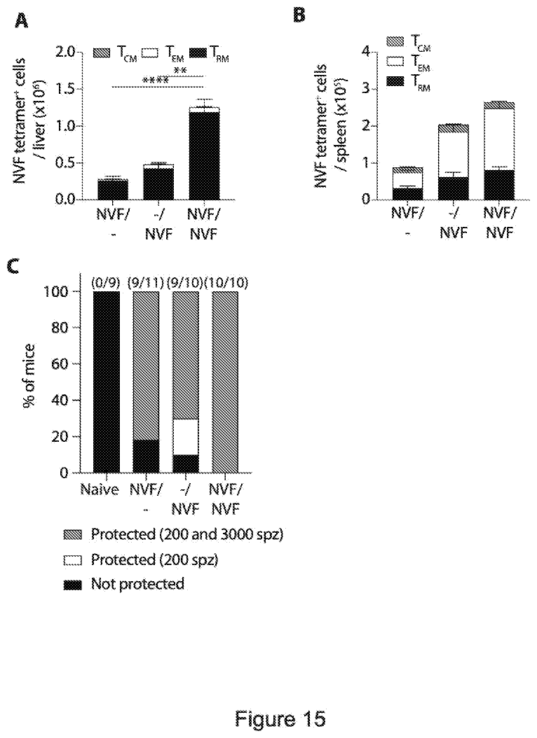

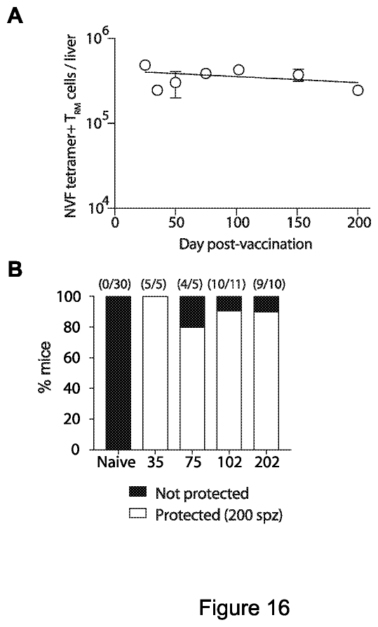

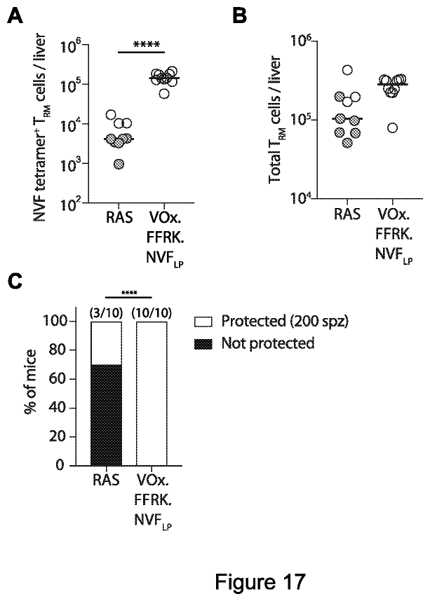

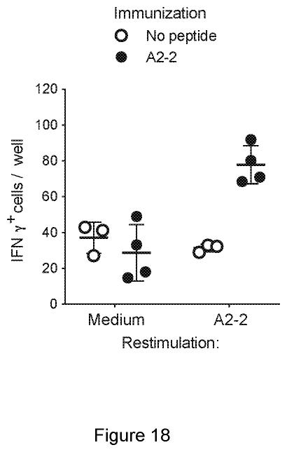

However, as set out in Example 1, co-administration of several such adjuvants with a fusion protein comprising an anti-Clec9A-NVF conjugate found that only CpG oligonucleotide had any marked effect on liver T_{RM} cell levels (see FIG. 1).

CpG adjuvant was also found to be effective in a prime-and-trap vaccination strategy using the same fusion protein and recombinant adeno-associated viral vectors. However, substitution of CpG with the adjuvant α-Galactosyl Ceramine (α-GalCer) gave a much poorer liver T_{RM} cell response in prime-and-trap vaccination (see FIG. 3), suggesting that the relationship is more complicated.

α-Gal-Cer is a glycolipid antigen that binds to CD1d receptors on antigen presenting cells (APCs) activating Type 1 Natural Killer T (NKT) cells. NKT cells are known to provide an adjuvant effect in a vaccination setting in a number of animal models (Gonzalez-Aseguinolaza G, 2002) (Fujii S, 2003) (Hermans I F, 2003) (Anderson R J, 2017).

NKT cells are typically found in large numbers in the liver and spleen, and respond rapidly to α-GalCer presented in the context of CD1d molecules on APCs, thereby activating the APC through CD40L interactions and soluble factors (Fujii S, 2003). This NKT cell-mediated “licensing” promotes the release of chemokines to attract naïve T cells (Semmling V, 2010), and has been shown to be particularly effective in enhancing cross-priming of antigen-specific CD8⁺ T cells 20 (Hermans I F, 2003), including to a sporozoite vaccine (Gonzalez-Aseguinolaza G, 2002).

Importantly, NKT cells are not generally thought to be required for protection against malaria. RAS-vaccinated CD1d deficient mice, which lack NKT cells, show similar protection rates to wild-type mice after P. berghei sporozoite challenge (Widmann C, 1992). Furthermore, CD1d knockout mice or mice depleted of NKT cells display normal CD8⁺ T-cell responses following RAS vaccination indicating NKT cells do not provide help to CD8⁺ T cells (Li J, 2015) (Scheiblhofer S, 2017).

This expectation in the art underscores the surprising results described herein whereby the inventors have identified a class of compounds that is extremely effective at increasing populations of liver T_{RM} cells, and therefore can be used not only as adjuvants in malarial vaccination strategies, but potentially as vaccines per se.

The compounds described herein comprise a modified α-GalCer structure conjugated via a cleavable linker to a peptide epitope. The α-GalCer component is α-GalCer modified by N→O acyl migration of the hexacosanoyl moiety under acidic conditions to expose a free amino group convenient for chemical linkage of the peptide.

This modified α-GalCer structure is linked to the peptide epitope via a linker that immolates after cleavage, allowing the glycolipid and peptide components to be cleanly separated from the linker intracellularly once the conjugate is acquired by APCs.

A reverse O→N acyl migration is then favored within the glycolipid structure, forming α-GalCer, which can then be presented via CD1d to NKT cells.

This general glycolipid-peptide conjugate approach to vaccine design has shown promise in mouse models of cancer and influenza infection. However, the inability of α-GalCer to increase liver T_{RM} cell populations makes it surprising that α-GalCer-peptide conjugates could be efficacious as malaria vaccines, in which sterile protection requires the generation of a large population of malaria-specific T_{RM} cells in the liver.

### 5.3 Compounds of the Invention

The invention relates to a specific class of α-GalCer-peptide conjugates that has been found to be highly effective in increasing the number of liver T_{RM} cells.

In one aspect the invention relates to a compound of Formula I:

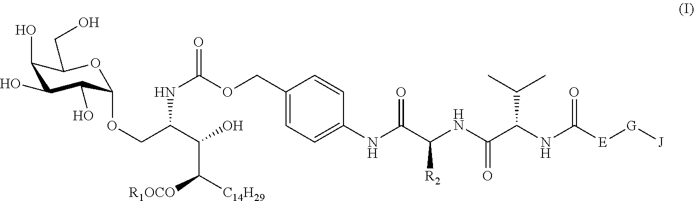

wherein
R₁ is (C₁₇-C₂₅)alkyl,
R₂ is the side-chain for alanine or citrulline,
E is a linker selected from S or Ox

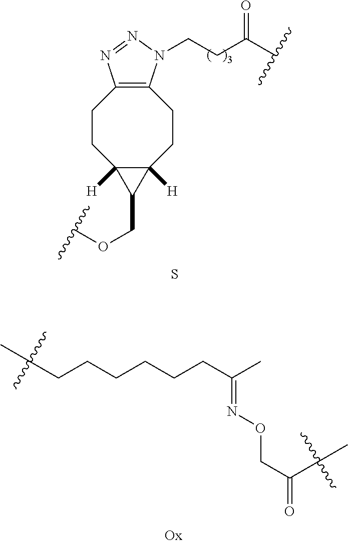

G is absent or is an amino acid sequence selected from the group consisting of FFRK (SEQ ID NO: 1), FKFL (SEQ ID NO: 16) and GFLG (SEQ ID NO: 17); and
J is a peptide antigen.

The compounds of the invention include a valine-citrulline or valine-alanine group.

In one embodiment, the compound of Formula I is a compound of Formula V.S.G.J:

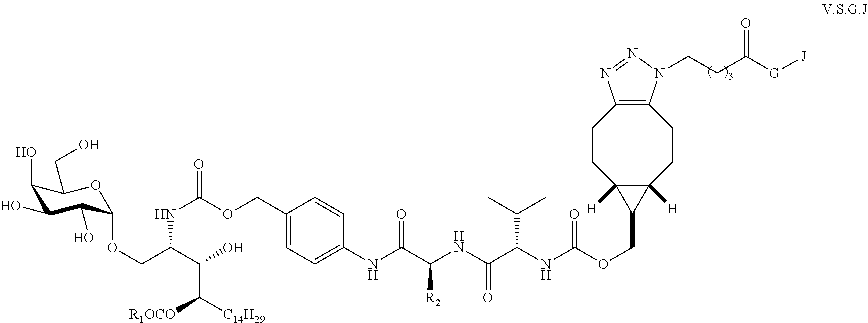

wherein
R₁ is (C₁₇-C₂₅)alkyl,
R₂ is the side-chain for alanine or citrulline,
G is absent or is an amino acid sequence selected from the group consisting of FFRK (SEQ ID NO: 1), FKFL (SEQ ID NO: 16) and GFLG (SEQ ID NO: 17); and
J is a peptide antigen.

In one embodiment, the compound of Formula I is a compound of Formula V.Ox.G.J:

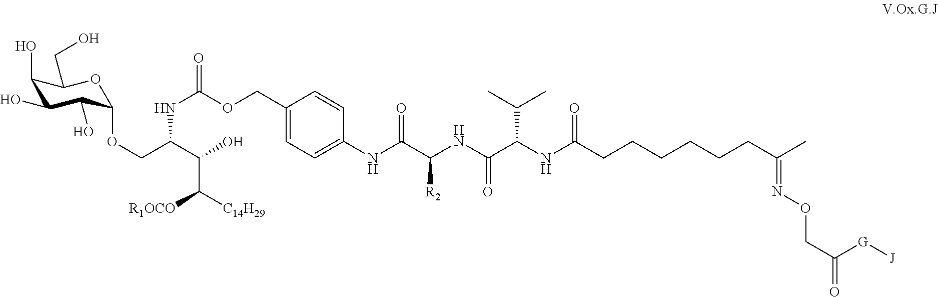

wherein
R₁ is (C₁₇-C₂₅)alkyl,
R₂ is the side-chain for alanine or citrulline,
G is absent or is an amino acid sequence selected from the group consisting of FFRK (SEQ ID NO: 1), FKFL (SEQ ID NO: 16) and GFLG (SEQ ID NO: 17); and
J is a peptide antigen.

The designations V.S.G.J and V.Ox.G.J are used to distinguish conjugates of the invention comprising the linker groups SPAAC (strain-promoted alkyne-azide cycloadditions) and oxime, respectively.

The designations V.S.G.J and V.Ox.G.J indicate compounds where R₁ is C25, unless otherwise specified.

In one embodiment R₁ is (C₁₉-C₂₅)alkyl, preferably (C₂₁-C₂₅)alkyl, more preferably (C₂₅)alkyl. In one embodiment, (Cₓ-C_{y})alkyl means a straight-chain saturated hydrocarbon radical of x to y carbon atoms.

In one embodiment R₁ is (C₁₉-C₂₅)alkyl, preferably (C₂₁-C₂₅)alkyl, more preferably (C₂₅)alkyl, where alkyl is a straight-chain saturated hydrocarbon radical.

In one embodiment R₂ is the side chain of citrulline.

In one embodiment, G is FFRK (SEQ ID NO: 1), FKFL (SEQ ID NO: 16) or GFLG (SEQ ID NO: 17).

In one embodiment G is FFRK (SEQ ID NO: 1).

In one embodiment J comprises an epitope that binds an antigen expressed by an organism that infects at least one cell in the liver of the subject.

In one embodiment J comprises an epitope that binds an antigen expressed by an organism that infects the liver of the subject.

In one embodiment, the cell in the liver is a dendritic cell or macrophage.

In one embodiment the cell in the liver is an antigen presenting cell (APC).

In one embodiment the cell in the liver is a hepatocyte.

In one embodiment J comprises at least one epitope selected from the group consisting of Plasmodium epitopes.

In one embodiment the Plasmodium epitopes are selected from an epitope selected from the group consisting of DELDYENDIEKKICKMEKCS (SEQ ID NO: 22), ENDIEKKICKMEKCSSVFNV (SEQ ID NO: 23), GIQVRIKPGSANKPKDELDY (SEQ ID NO: 24), IKPGSANKPKDELDYENDIE (SEQ ID NO: 25), VTCGNGIQVRIKPGSANKPK (SEQ ID NO: 26), KPIVQYDNF (SEQ ID NO: 27), KPKDELDY (SEQ ID NO: 28), KPNDKSLY (SEQ ID NO: 29), KSKDELDY (SEQ ID NO: 30), MMRKLAILSV (SEQ ID NO: 31), MMRKLAILSVSSFLFVEALF (SEQ ID NO: 32), YLKKIKNSL (SEQ ID NO: 33), YLKKIQNSL (SEQ ID NO: 34), YLNKIQNSL (SEQ ID NO: 35), ASKNKEKAL (SEQ ID NO: 36), GIAGGLALL (SEQ ID NO: 37), HLGNVKYLV (SEQ ID NO: 38), KNKEKALII (SEQ ID NO: 39), KSLYDEHI (SEQ ID NO: 40), LRKPKHKKL (SEQ ID NO: 41), MINAYLDKL (SEQ ID NO: 42), MPNDPNRNV (SEQ ID NO: 43), LLMDCSGSI (SEQ ID NO: 44), MPNNPNRNV (SEQ ID NO: 45), HLGNKYLV (SEQ ID NO: 46), FILVNLLIFH (SEQ ID NO: 47), ALFFIIFNK (SEQ ID NO: 48), FLIFFDLFLV (SEQ ID NO: 49), GLIMVLSFL (SEQ ID NO: 50), GLLGNVSTV (SEQ ID NO: 51), GVSENIFLK (SEQ ID NO: 52), HVLSHNSYEK (SEQ ID NO: 53), ILSVSSFLFV (SEQ ID NO: 54), KILSVFFLA (SEQ ID NO: 55), LACAGLAYK (SEQ ID NO: 56), LLACAGLAY (SEQ ID NO: 57), MPLETQLAI (SEQ ID NO: 58), QTNFKSLLR (SEQ ID NO: 59), TPYAGEPAPF (SEQ ID NO: 60), VLAGLLGNV (SEQ ID NO: 61), VLLGGVGLVL (SEQ ID NO: 62), VTCGNGIQVR (SEQ ID NO: 63), EPSDKHIKEY (SEQ ID NO: 64), DLDEPEQFRL (SEQ ID NO: 65), IMVLSFLFL (SEQ ID NO: 66), KLKKIKNSI (SEQ ID NO: 67), KLQEQQSDL (SEQ ID NO: 68), KLRKPKHKKL (SEQ ID NO: 69), NLNDNAIHL (SEQ ID NO: 70), NMPNDPNRNV (SEQ ID NO: 71), SLKKNSRSL (SEQ ID NO: 72), TLRKPKHKKL (SEQ ID NO: 73), PSDKHIKEYLNKIQNSLSTE (SEQ ID NO: 74), IKEYLNKIQNSLSTEWSPCS (SEQ ID NO: 75), IKPGSANKPKDELDYANDIE (SEQ ID NO: 76), DLLEEGNTL (SEQ ID NO: 77), ILYISFYFI (SEQ ID NO: 78), RLEIPAIEL (SEQ ID NO: 79), VLDKVEETV (SEQ ID NO: 80), APFISAVAA (SEQ ID NO: 81), LLACAGLLAYK (SEQ ID NO: 82), AILSVSSFLF (SEQ ID NO: 83), DKHIKEYLNKIQNSL (SEQ ID NO: 84), EALFQEYQCYGSSSN (SEQ ID NO: 85), EKKICKMEKCSSVFN (SEQ ID NO: 86), ELNYDNAGTNLYNEL (SEQ ID NO: 87), FLFVEALF (SEQ ID NO: 88), FLFVEALFQEYQCYG (SEQ ID NO: 89), FVEALFQEY (SEQ ID NO: 90), KCSSVFNVVNSSIGL (SEQ ID NO: 91), KEYLNKIQNSLSTEW (SEQ ID NO: 92), LFVEALFQEY (SEQ ID NO: 93), LIMVLSFLF (SEQ ID NO: 94), QEYQCYGSSSNTRVL (SEQ ID NO: 95), SFLFVEALF (SEQ ID NO: 96), SSIGLIMVLSFLFLN (SEQ ID NO: 97), SSNTRVLNELNYDNA (SEQ ID NO: 98), SVFNVVNSSI (SEQ ID NO: 99), SVSSFLFVEA (SEQ ID NO: 100), SVSSFLFVEALFQEY (SEQ ID NO: 101), TNLYNELEMNYYGKQ (SEQ ID NO: 102), VFNVVNSSI (SEQ ID NO: 103), VFNVVNSSIGLIMVL (SEQ ID NO: 104), VNSSIGLIMVLSFLF (SEQ ID NO: 105), CEIFNVKPTCLINNS (SEQ ID NO: 106), EMRHFYKDNKYVKNL (SEQ ID NO: 107), ETQKCEIFNVKPCL (SEQ ID NO: 108), FEFTYMINF (SEQ ID NO: 109), FKADRYKSHGKGYNW (SEQ ID NO: 110), HPKEYEYPL (SEQ ID NO: 111), KLVFELSA (SEQ ID NO: 112), NEFPAIDLF (SEQ ID NO: 113), NEVVVKEEY (SEQ ID NO: 114), NQYLKDGGFAFPTE (SEQ ID NO: 115), SDVYRPINEH (SEQ ID NO: 116), TLDEMRHFYK (SEQ ID NO: 117), TQKCEIFNV (SEQ ID NO: 118), YEYPLHQEH (SEQ ID NO: 119), TLDEMRHFY (SEQ ID NO: 120), KSHGKGYNW, (SEQ ID NO: 121) NSTCRFFVCK (SEQ ID NO: 122), YKSHGKGYNW (SEQ ID NO: 123), KSRGKGYNW (SEQ ID NO: 124), DASKNKEKALIIIKS (SEQ ID NO: 125), IRLHSDASKNKEKAL (SEQ ID NO: 126), KNKEKALI (SEQ ID NO: 127), LPMSNVKNV (SEQ ID NO: 128), LSMSNVKNV (SEQ ID NO: 129), LTMSNVKNV (SEQ ID NO: 130), ATSVLAGL (SEQ ID NO: 131), EPKDEIVEV (SEQ ID NO: 132), GLLNKLENI (SEQ ID NO: 133), KLEELHENV (SEQ ID NO: 134), MEKLKELEK (SEQ ID NO: 135), KLKEFIPKV (SEQ ID NO: 136), ALLACAGLAYKFVVP (SEQ ID NO: 137), ALLQVRKHLNDRINR (SEQ ID NO: 138), APFDETLGEEDKDLD (SEQ ID NO: 139), CEEERCLPKREPLDV (SEQ ID NO: 140), CLPKREPLDVPDEPE (SEQ ID NO: 141), ENVKNVIGPFMKAVC (SEQ ID NO: 142), EVDLYLLMDCSGSIR (SEQ ID NO: 143), LLSTNLPYGKTNLTD (SEQ ID NO: 144), LPYGKTNLTDALLQV (SEQ ID NO: 145), MNHLGNVKYLVIVFL (SEQ ID NO: 146), TLGEEDKDLDEPEQF (SEQ ID NO: 147), and TNLTDALLQVRKHLN (SEQ ID NO: 148).

In one embodiment J comprises at least one epitope selected from the group consisting of Hepatitis virus epitopes, preferably Hepatitis A, B, C and/or D epitopes, preferably HBV epitopes.

In one embodiment the antigen expressed by the organism that infects the liver, or that infects at least one cell in the liver of the subject is a Plasmodium antigen.

In one embodiment the Plasmodium antigen is expressed on a macrophage or a dendritic cell. In one embodiment the macrophage or dendritic cell is an antigen presenting cell.

In one embodiment the Plasmodium antigen is expressed on a hepatocyte.

In one embodiment the antigen expressed by the organism that infects the liver, or that infects at least one cell in the liver of the subject, is a Hepatitis virus antigen.

In one embodiment the Hepatitis virus antigen is expressed on a macrophage or a dendritic cell. In one embodiment the macrophage or dendritic cell is an antigen presenting cell.

In one embodiment the Hepatitis virus antigen is expressed on a hepatocyte.

In one embodiment the Hepatitis virus antigen is a Hepatitis A, B, C and/or D antigen, preferably an HBV antigen.

In one embodiment J comprises at least one amino acid residue flanking the at least one epitope.

In one embodiment J comprises at least one amino acid residue flanking the N-terminal and the C-terminal of the at least one epitope.

In one embodiment J comprises from one to ten amino acid residues flanking the N-terminal or the C-terminal of the at least one epitope.

In one embodiment J comprises one, preferably two, preferably three, preferably four amino acid residues flanking the N-terminal or C-terminal of the epitope, or both.

In one embodiment J comprises four amino acid residues flanking the N-terminal or C-terminal of the at least one epitope, or both. Preferably J comprises four amino acid residues flanking the N-terminal and four amino acid residues flanking the C-terminal of the epitope. Preferably J comprises HSLS or LERD or both.

In one embodiment the at least one epitope comprises a flanking amino acid sequence comprising of HSLS and a flanking amino acid sequence comprising of LERD.

In one embodiment the at least one epitope comprises a flanking amino acid sequence consisting of HSLS and a flanking amino acid sequence consisting of LERD.

In one embodiment the at least one epitope comprises an N-terminal amino acid sequence comprising of HSLS and a C-terminal amino acid sequence comprising of LERD.

In one embodiment the at least one epitope comprising an N-terminal amino acid sequence consisting of HSLS and a C-terminal amino acid sequence consisting of LERD.

In some embodiments where J comprises more than one epitope, each epitope may comprise flanking amino acid residues as set out herein for “at least one epitope”.

In one embodiment the peptide antigen comprises an N-terminal spacer.

In one embodiment the N-terminal spacer comprises one amino acid residue, preferably two, three, four, five, six, seven, eight, nine, preferably ten or more amino acid residues.

In one embodiment the N-terminal spacer comprises three amino acid residues, preferably the three amino acid residues are AAA.

In one embodiment, J is a Plasmodium spp. antigen, preferably a P. berghei antigen, preferably a P. berghei ANKA antigen.

In one embodiment, G is FFRK (SEQ ID NO: 1).

In one embodiment, J is selected from the group consisting of NVYDFNLL (SEQ ID NO: 2) (NVY_{SP}), AAAHSLSNVYDFNLLLERD (SEQ ID NO: 3) (NVY_{LP}), NVFDFNNL (SEQ ID NO: 4) (NVF_{SP}) and AAASTNVFDFNNLS (SEQ ID NO: 5) (NVF_{LP}), DNQKDIYYITGESINAVS (SEQ ID NO: 6), AAALTSALLNVDNLIQ (SEQ ID NO: 7), STNVFDFNNLS (SEQ ID NO: 8), EIYIFTNI (SEQ ID NO: 13), ILNSGLLAV (SEQ ID NO: 18), TKILNSGLLAVVG (SEQ ID NO: 19), and HSLSILNSGLLAVLERD (SEQ ID NO: 20).

In one embodiment, J is selected from the group consisting of NVYDFNLL (SEQ ID NO: 2)(NVY_{SP}), AAAHSLSNVYDFNLLLERD (SEQ ID NO: 3)(NVY_{LP}), NVFDFNNL (SEQ ID NO: 4) (NVF_{SP}) and AAASTNVFDFNNLS (SEQ ID NO: 5)(NVF_{LP}).

In one embodiment, J is selected from the group consisting of ILNSGLLAV (SEQ ID NO: 18), TKILNSGLLAVVG (SEQ ID NO: 19), and HSLSILNSGLLAVLERD (SEQ ID NO: 20).

In one embodiment the compound of Formula I is selected from the group consisting of

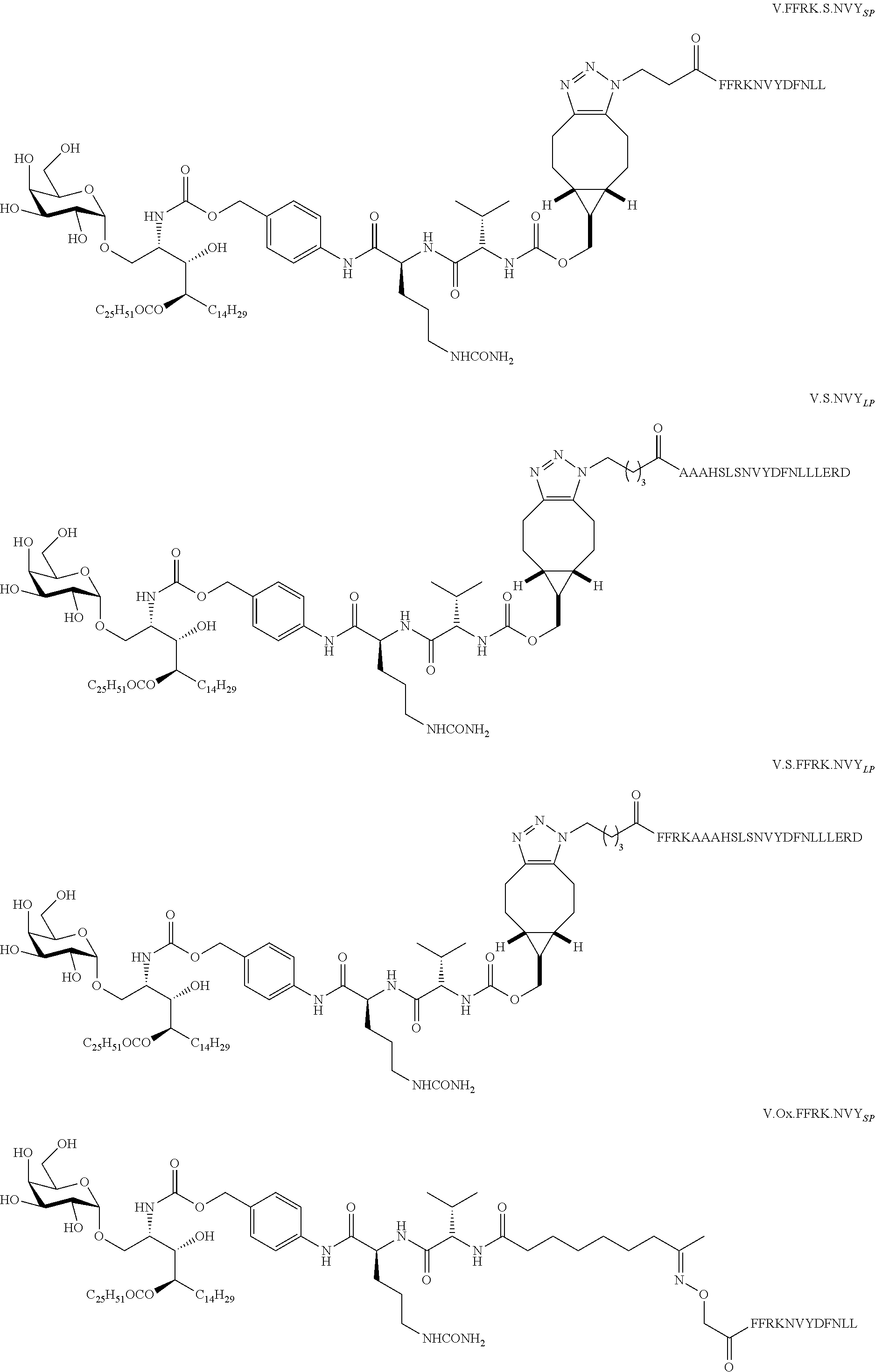

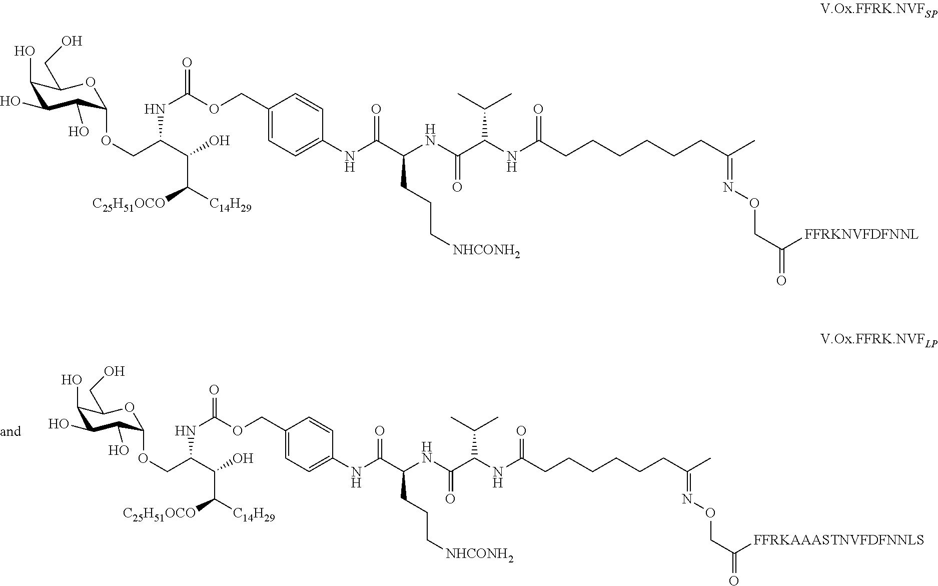

In one aspect the invention relates to a pharmaceutical composition comprising a compound of Formula I and at least one pharmaceutically acceptable carrier or excipient.

In one aspect the invention relates to a vaccine comprising a compound of Formula I and at least one pharmaceutically acceptable carrier or excipient.

Pharmaceutical compositions and vaccines as described herein can be administered topically, orally or parenterally, preferably parenterally.

For example, the pharmaceutical compositions and vaccines described herein can be injected parenterally, such as, intravenously, intramuscularly or subcutaneously. For parenteral administration, the compositions and vaccines can be formulated as known in the art, for example, in a sterile aqueous solution or suspension that optionally comprises other substances, such as salt or glucose, but not limited thereto.

In one non-limiting example of parenteral administration a composition or vaccine of the invention is formulated as an injectable composition, and can be prepared in the form of a sterile solution or emulsion.

The compositions and vaccines described herein can be presented in unit dosage form and can be prepared by any of the methods well known in the art of pharmacy. The term “unit dosage form” means a single dose wherein all active and inactive ingredients are combined in a suitable system, such that the patient or person administering the drug can open a single container or package with the entire dose contained therein and does not have to mix any components together from two or more containers or packages. Any examples of unit dosage forms are not intended to be limiting in any way, but merely to represent typical examples in the pharmacy arts of unit dosage forms.

### 5.4 Uses of the Compounds of the Invention

As discussed above, the inventors have surprisingly found that the select class of compounds are extremely effective at increasing the number of liver T_{RM} cells in a subject and therefore are effective malaria vaccines per se.

The inventors have shown that the conjugate compounds of the invention act to increase the number of liver T_{RM} cells in mice as shown in FIGS. 4, 5 and 6 (see Example 6).

Based on their inventive contribution as detailed herein, the inventors believe that although a person of skill in the art might expect to see an increase in the number of liver T_{RM} cells in a subject in response to an antigenic challenge that increases the number of liver Trm cells in a subject, the person of skill would not expect to see the surprising relative increase in the number of liver T_{RM} cells observed in the subjects administered a compound of the invention (as compared to the control subjects). Without wishing to be bound by theory the inventors believe that it is this surprising increase in the relative numbers of liver T_{RM} cells that affords the levels of prophylaxis observed in subsequent trials.

Moreover as shown in Example 5, the effects of known adjuvants on the generation of liver T_{RM} cells are not predictable, making it even more surprising to the inventors that the conjugates of the invention are able to generate such a strong liver T_{RM} cell response.

To exquisitely demonstrate the protective effect of liver T_{RM} cells against malarial challenge, the inventors show that vaccination with the conjugate compound V.FFRK.NVY_{SP} protects a portion of mice from malaria (FIGS. 4, 7, 8, 9, 10, 12 and 14).

In a further demonstration of the efficacy of the compounds of the invention as vaccines, the inventors show that the conjugate compound V.FFRK.NVY_{SP} is an effective prime-boost vaccine (FIG. 8).

The inventors have also determined that the conjugate compounds of the invention can be modified to contain peptide flanking residues (to one side or the other of an epitope sequence in a peptide antigen as described, herein, or both), and that such modifications can increase the number of liver T_{RM} cells generated as compared to compounds of the invention comprising an antigenic peptide comprising an epitope lacking peptide flanking residues.

The inventors also show that a particular subset of the compounds of the invention, that are conjugates linked as described herein using oxime chemistry, elicit sterile immunity against Plasmodium.

For example, the protective effect of liver T_{RM} cells against malarial challenge is greatly enhanced when multiple doses of the conjugate compound V.Ox.FFRK.NVF_{LP} [NVFDFNNL (SEQ ID NO: 2) with flanking sequences [AAASTNVFDFNNLS (SEQ ID NO: 5)] is used to vaccinate mice (FIG. 15).

Additionally, the inventors have shown that the protective effect liver T_{RM} cells against malarial challenge provided by vaccination with a conjugate compound as described herein is long term, with a single dose of a conjugate compound vaccine providing protection at least 200 days after vaccination (FIG. 16).

Moreover, a single vaccination with a conjugate compound as described herein is shown to be more effective than the current gold standard malarial vaccine based on radiation attenuated sporozites (RAS) (FIG. 17).

Furthermore, described herein is an epitope specific CD8⁺ T cell response (FIG. 18) that is triggered by a P. falciparum RPL6 epitope, ILNSGLLAV (SEQ ID NO: 18). Without wishing to be bound by theory, the inventors believe that this epitope can be used as “J” in conjugate compounds as disclosed herein (wherein “J” comprises, consists essentially of, or consists of any one of SEQ ID NO: 18, 19 or 20). When used as a vaccine, such a conjugate compound would be expected act to increase in the relative numbers of liver T_{RM} cells in a vaccinated subject, thereby providing at least some level of prophylaxis against Plasmodium.

Taken together, the data disclosed herein shows that the compounds of the invention, particularly when comprising peptide flanking residues and oxime linkages, are effective malarial vaccines and can be used directly or in prime-boost regimens to elicit a T_{RM} sterile protection against malaria.

### Hepatic Infections

Malaria is not the only disease or condition mediated by hepatic infection. Hepatic infections are caused by various parasites including protists, bacteria and viruses that can infect the liver causing inflammation that can act to reduce liver function. Certain viruses that damage the liver can also be spread in blood or semen, contaminated food or water, or close contact with a person who is infected. Common viruses that cause hepatic infection are the hepatitis viruses including Hepatitis A (HBA), Hepatitis B (HBV), Hepatitis C (HBC) and Hepatitis D (HBD).

Regarding HBV, Pallet et al. (Pallett, 2017) demonstrate the presence of an abundant population of liver resident HBV-specific memory T cells that display a distinct phenotype and are strategically positioned for site-specific immune surveillance and immune responses.

Pallet et al. note that it is critical to further decipher the role of liver resident HBV-specific memory T cells to develop effective immunotherapeutic approaches for chronic HBV infection. However, Pallet et al. provide no guidance as to how this might be done.

In view of the disclosure provided herein, the inventors believe that a skilled person in the art recognizes that the compounds of the invention and as described herein can provide a subject with a level of prophylaxis against any hepatic infection by modifying the peptide antigen to comprise at least one epitope from a given infective agent, where that epitope(s) increases the number of liver T_{RM} cells.

In particular, the inventors believe that a skilled person in the art can, based on the disclosure provide herein, use the compounds of the invention and described herein as a vaccine against HBV by modifying the peptide antigen to comprise an HBV epitope that activates a population of CD8⁺ T cells that increase the number of liver T_{RM} cells to an amount that is sufficient to provide at least some level of prophylaxis against, or to prevent, HBV infection.

Therefore, the present disclosure is not limited solely to a method of preventing malaria, but encompasses more broadly a method of increasing the number of liver T_{RM} cells in a subject to an amount that prevents the infection of at least one cell in the liver of a subject.

### Medical Uses

In another aspect the invention relates to a method of increasing the number of liver T_{RM} cells in a subject comprising administering a compound of Formula I to the subject.

In one embodiment the compound of Formula I is as defined herein for any of the embodiments set out for and encompassed within the compound aspects of the invention.

In one embodiment the number of liver T_{RM} cells in the subject is increased relative to a control subject.

In one embodiment the number of liver T_{RM} cells in the subject is increased relative to the number of liver T_{RM} cells in the subject before administration.

In one embodiment the number of liver T_{RM} cells is increased by about 10 times, preferably by about 100 times, preferably by about 1000 times, preferably by about 10,000 times, preferably by about 100,000 times, preferably by about 1,000,000 times relative to any increase in the number of liver T_{RM} cells observed in a control subject or relative to the number of liver T_{RM} cells in the subject before administration.

In one embodiment the number of liver T_{RM} cells is increased by at least 10 times, preferably by at least 100 times, preferably by at least 1000 times, preferably by at least 10,000 times, preferably by at last 100,000 times, preferably by at least 1,000,000 times relative to any increase observed in a control subject or relative to the number of liver T_{RM} cells in the subject before administration.

In one embodiment the number of liver T_{RM} cells is increased by at least 1×10¹, preferably by at least 1×10², preferably by at least 1×10³, preferably by at least 1×10⁴, preferably by at least 1×10⁵, preferably by at least 1×10⁶ cells relative to any increase observed in a control subject or relative to the number of liver T_{RM} cells in the subject before administration.

In one embodiment the number of liver T_{RM} cells is increased by about 1×10¹, preferably by about 1×10², preferably by about 1×10³, preferably by about 1×10⁴, preferably by about 1×10⁵, preferably by about 1×10⁶ cells relative to any increase observed in a control subject or relative to the number of liver T_{RM} cells in the subject before administration.

In one embodiment the number of liver T_{RM} cells is increased by about 10%, preferably about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, preferably about 100% relative to any increase observed in a control subject or relative to the number of liver T_{RM} cells in the subject before administration.

In one embodiment the number of liver T_{RM} cells is increased by at least 10%, preferably at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, preferably at least 100% relative to any increase observed in a control subject or relative to the number of liver T_{RM} cells in the subject before administration.

In one embodiment the number of liver T_{RM} cells is increased by an number that is sufficient to provide at least some level of prophylaxis to the subject.

In one embodiment the prophylaxis provided lasts about 60 days, preferably about 70 days, 80 days, 90 days, 100 days, 110 days, 120 days, 130 days, 140 days, 150 days, 160 days, 170 days, 180 days, 190 days, preferably about 200 days.

In one embodiment the prophylaxis provided lasts at least 60 days, preferably at least 70 days, 80 days, 90 days, 100 days, 110 days, 120 days, 130 days, 140 days, 150 days, 160 days, 170 days, 180 days, 190 days, preferably at least 200 days.

In one embodiment the prophylaxis provided is at least 40%, preferably at least 50% greater, preferably at least 60% greater, preferably at least 70% greater than the prophylaxis provided to a subject that has been administered a malarial vaccine comprising radiation attenuated sporozoites (RAS).

In one embodiment the prophylaxis provided is about 40%, preferably about 50% greater, preferably about 60% greater, preferably about 70% greater than the prophylaxis provided to a subject that has been administered a malarial vaccine comprising radiation attenuated sporozoites (RAS).

In one embodiment the liver T_{RM} cells express at least one cell surface marker selected from the group consisting of one of CD8, CD44, and CD69, preferably at least two, preferably all three cell surface markers.

In one embodiment the liver T_{RM} cells express CD8, CD44 and CD69.

In one embodiment the liver T_{RM} do not express KLRG1 or CX3CR1.

In one embodiment the liver T_{RM} cells are CD8⁺Ly5.1⁺ CD44⁺ CD69⁺ CD62L^{low}.

In one embodiment the liver T_{RM} cells are CD69+CD62L^{low} after gating on CD8+, CD44^{high}.

In one embodiment the compound is formulated for administration with at least one pharmaceutically acceptable carrier or excipient.

In one embodiment administration comprises systemic administration, preferably parenteral administration. In one embodiment parenteral administration is by injection.

In one embodiment administration comprises administering the compound using a prime boost regimen, preferably a heterologous prime boost regimen.

In one embodiment the prime boost regimen comprises a first administration (A1) and a second administration (A2).

In one embodiment A2 is separated from A1 by at least 7, at least 14, at least 21, at least 28, at least 35, at least 42, at least 49, at least 56, at least 63, at least 70, at least 77, at least 84, at least 93, at least 100 days.

In one embodiment A2 is separated from A1 by about 7, about 14, about 21, about 28, about 35, about 42, about 49, about 56, about 63, about 70, about 77, about 84, about 93, about 100 days.

In one embodiment A2 is separated from A1 by about 30 days.

In one embodiment A2 is separated from A1 by at least 30 days.

In one embodiment A2 is separated from A1 by 30 days.

In one embodiment the method comprises a third administration A3.

In one embodiment A1 comprises administering a therapeutically effective amount of the compound.

In another aspect the invention relates to the use of a compound of Formula I in the manufacture of a medicament for increasing the number of liver T_{RM} cells in a subject.

In one embodiment the medicament comprises an effective amount of the compound of Formula I.

In one embodiment the effective amount is a therapeutically effective amount.

In one embodiment the medicament is formulated for administration, or is in a form for administration, to a subject in need thereof.

In one embodiment the medicament is in a form for, or is formulated for parenteral administration. In one embodiment parenteral administration is injection, intravenous drip or infusion, inhalation, insufflation or intrathecal or intraventricular administration.

In one embodiment the medicament is formulated for, or is in the form of an injectable composition, or when administered, is administered by injection. In one embodiment the medicament is formulated for, or is in the form of an intravenous composition, or when administered, is administered intravenously.

In one embodiment the medicament is in a form for, or is formulated for, parenteral administration in any appropriate solution, preferably in a sterile aqueous solution which may also contain buffers, diluents and other suitable additives.

In one embodiment the medicament is in a form for, or is formulated for use in a prime boost regimen.

In another aspect the invention relates a compound of Formula I for use for increasing the number of liver T_{RM} cells in a subject.

Specifically contemplated as embodiments of the invention directed to a compound of Formula I for use for increasing the number of liver T_{RM} cells in a subject, and the use of a compound of Formula I in the manufacture of a medicament for increasing the number of liver T_{RM} cells in a subject, are all of the embodiments set out and encompassed within the aspects of the invention that are the compound of Formula I, and a method of increasing the number of liver T_{RM} cells in a subject.

In another aspect the invention relates to a method of inducing an immune response that will reduce liver cell infection in a subject comprising administering a compound of Formula I to the subject.

In one embodiment the immune response reduces liver cell infection to the point of no ongoing infection.

In one embodiment the immune response prevents blood-stage infection.

In one embodiment the immune response prevents blood stage infection for about 60 days, preferably about 70 days, 80 days, 90 days, 100 days, 110 days, 120 days, 130 days, 140 days, 150 days, 160 days, 170 days, 180 days, 190 days, preferably about 200 days.

In one embodiment the immune response prevents blood stage infection at least 60 days, preferably at least 70 days, 80 days, 90 days, 100 days, 110 days, 120 days, 130 days, 140 days, 150 days, 160 days, 170 days, 180 days, 190 days, preferably at least 200 days.

In one embodiment the immune response prevents the infection of erythrocytes.

In one embodiment the immune response prevents infection of erythrocytes for about 60 days, preferably about 70 days, 80 days, 90 days, 100 days, 110 days, 120 days, 130 days, 140 days, 150 days, 160 days, 170 days, 180 days, 190 days, preferably about 200 days.

In one embodiment the immune response prevents infection of erythrocytes for at least 60 days, preferably at least 70 days, 80 days, 90 days, 100 days, 110 days, 120 days, 130 days, 140 days, 150 days, 160 days, 170 days, 180 days, 190 days, preferably at least 200 days.

In one embodiment the infection is reduced by at least 10%, preferably at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, preferably by 100% as compared to a control subject.

In one embodiment the infection is reduced by at least 10%, preferably at least 15%, at least 20%, at least 25%, at least 30%, at least 35%, at least 40%, at least 45%, at least 50%, at least 55%, at least 60%, at least 65%, preferably at least 70% as compared to a subject that has been administered a malarial vaccine comprising radiation attenuated sporozoites (RAS).

In one embodiment the infection is reduced by about 10%, preferably about 15%, about 20%, about 25%, about 30%, about 35%, about 40%, about 45%, about 50%, about 55%, about 60%, about 65%, preferably about 70% as compared to a subject that has been administered a malarial vaccine comprising radiation attenuated sporozoites (RAS).

In one embodiment the liver cell infection is a Plasmodium infection.

In one embodiment the liver cell infection is a hepatitis virus infection, preferably a hepatitis virus A, B, C and/or D infection, preferably an HBV infection.

In another aspect the invention relates a compound of Formula I for use for inducing an immune response that will reduce liver cell infection in a subject.

In another aspect the invention relates to the use of a compound of Formula I in the manufacture of a medicament for inducing an immune response that will reduce liver cell infection in a subject.

Specifically contemplated as embodiments of the invention directed to a method of inducing an immune response that will reduce liver cell infection in a subject, a compound of Formula I for use for inducing an immune response that will reduce liver cell infection in a subject, and the use of a compound of Formula I in the manufacture of a medicament for inducing an immune response that will reduce liver cell infection in a subject, are all of the embodiments set out and encompassed within the aspects of the invention that are the compound of Formula I, a method of increasing the number of liver T_{RM} cells, a compound of Formula I for increasing the number of liver T_{RM} cells and the use of a compound of Formula I in the manufacture of a medicament for increasing the number of liver T_{RM} cells.

In another aspect the invention relates to a method of vaccinating a subject against a hepatic infection comprising administering a compound of Formula I to the subject.

In one embodiment vaccinating the subject comprises providing immunity from the infection in at least 10%, preferably at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, preferably all of the vaccinated subjects as compared to a control subject. Preferably the vaccinating the subject provides sterile protection to the subject as compared to a control subject.

In another aspect the invention relates a compound of Formula I for use for vaccinating a subject against a hepatic infection.

In another aspect the invention relates to the use of a compound of Formula I in the manufacture of a medicament for vaccinating a subject against a hepatic infection.

A person skilled in the art will be able to choose the appropriate mode of administration of the medicament with reference to the literature and as described herein. By way of non-limiting example, parenteral administration comprising injection or intravenous administration would be preferred for vaccination against hepatic infection.

Specifically contemplated as embodiments of the invention directed to a method of vaccinating a subject against hepatic infection, a compound of Formula I for use for vaccinating and the use of a compound of Formula I in the manufacture of a medicament for vaccinating, are all of the embodiments set out and encompassed within the aspects of the invention that are the compound of Formula I, a method of increasing the number of liver T_{RM} cells, a compound of Formula I for increasing the number of liver T_{RM} cells, the use of a compound of Formula I in the manufacture of a medicament for increasing the number of liver T_{RM} cells, a method of inducing an immune response that will reduce liver cell infection in a subject, a compound of Formula I for use for inducing an immune response that will reduce liver cell infection in a subject, and the use of a compound of Formula I in the manufacture of a medicament for inducing an immune response that will reduce liver cell infection in a subject.

Various aspects of the invention will now be illustrated in non-limiting ways by reference to the following examples.

### 6. EXAMPLES

### 6.1 Materials and Methods

### Mice

C57BL/6 (B6), OT-I (Hogquist K A, 1994), PbT-I (Lau L S, 2014) and CD1d^{-/-} mice (Exley M A, 2003) were bred and maintained at the Department of Microbiology and Immunology, The University of Melbourne, or the BRU, Malaghan Institute of Medical Research, New Zealand. All mice used were 6-12 weeks of age and littermates of the same sex were randomly assigned to experimental groups. Animals used for the generation of the sporozoites were 4-5 week-old male Swiss Webster mice purchased from the Monash Animal Services (Melbourne, Victoria, Australia) and housed at 22° to 25° C. on a 12 hr light/dark cycle at the School of Biosciences, The University of Melbourne, Australia.

### Plasmodium Infection

Anopheles stephensi mosquitoes (STE2, MRA-128, from BEI Resources) were reared in an Australian Biosecurity (Department of Agriculture and Water Resources) approved insectary. The conditions were maintained at 27° C. and 75-80% humidity with a 12 h light and dark photo-period in filtered drinking water (Frantelle beverages, Australia) and fed with Sera vipan baby fish food (Sera). The larvae were bred in plastic food trays (P.O.S.M Pty Ltd, Australia) containing 300 larvae, each with regular water changes every 3 days. Upon ecloding, the adult mosquitoes were transferred to aluminium cages (BioQuip Products, Inc. St. Rancho Dominguez, Calif., USA) and kept in a secure incubator (Conviron), in the insectary at the same temperature and humidity and maintained on 10% sucrose.

P. berghei ANKA wild-type Cl15cy1 (BEI Resources, NIAID, NIH: MRA-871, contributed by Chris J. Janse and Andrew P. Waters) were used to challenge vaccinated mice (Kimura K, 2013). Infections of naïve Swiss mice were carried out by i.p inoculation of PbA infected RBCs obtained from a donor mouse between the first and fourth passages from a cryopreserved stock. Parasitemia was monitored by Giemsa smear and exflagellation quantified 3 days post infection. 1 μL of tail prick blood was mixed with 100 μL of exflagellation media (RPMI [Invitrogen] supplemented with 10% v/v foetal bovine serum, pH 8.4), incubated for 15 min at 20° C., and exflagellation events per 1×10⁴ red blood cells were counted. A. stephensi mosquitoes were allowed to feed on anaesthetised mice once the exflagellation rate was assessed between 12-15 exflagellation events per 1×10⁴ red blood cells. 22 days after biting, salivary glands were dissected and checked for the presence of sporozoites.

Sporozoites were dissected from mosquito salivary glands (Ramakrishnan C, 2013), resuspended in cold PBS, and either left untreated for challenge experiments or irradiated with 20,000 rads using a gamma ⁶⁰Co source. For challenge experiments, 200 freshly dissected PbA sporozoites were injected i.v. as indicated. Mice were assessed for parasitemia at day 6, 7, 8, 10 and 12 using flow cytometry. Briefly, a drop of blood was collected from the mice and stained with Hoechst 33258 dye (ThermoFisher, Scoresby, Victoria, Australia) for 1 hr at 37° C. Samples were analyzed on a LSR Fortessa (BD Biosciences, San Jose, USA) using a violet laser (405 nm) to excite the dye. After gating on RBCs the percentage of Hoechst positive cells were measured. Values were compared to uninfected controls and typically values of >0.1% were considered positive for parasites. Mice positive for parasites on two consecutive days were euthanized. Mice were considered sterilely protected if they remained parasitemia-negative on day 12 after challenge.

### Synthesis of Glycolipid-Peptide Conjugates: General Synthesis Methods

Anhydrous solvents were obtained commercially. Air-sensitive reactions were carried out under Ar. Thin layer chromatography (TLC) was performed on aluminium sheets coated with 60 F₂₅₄ silica. Flash column chromatography was performed on Reveleris® silica cartridges (38.6 μm) or SiliCycle® silica gel (40-63 μm). NMR spectra were recorded on a Bruker 500 MHz spectrometer (Anderson R J, 2017). ¹H NMR spectra were referenced to tetramethylsilane at 0 ppm (internal standard) or to residual solvent peak (CHCl₃ 7.26 ppm, CHD₂OD 3.31 ppm, CHD₂(SO)CD₃ 2.50 ppm). ¹³C NMR spectra were referenced to tetramethylsilane at 0 ppm (internal standard) or to the deuterated solvent peak (CDCl₃ 77.0 ppm, CD₃OD 49.0 ppm, (CD₃)₂SO 39.5 ppm). CDCl₃-CD₃OD solvent mixtures were always referenced to the methanol peak. High resolution electrospray ionization (ESI) mass spectra were undertaken on a Waters Q-TOF Premier™ Tandem Mass spectrometer fitted with a Waters 2795 HPLC. Semi-preparative HPLC and synthetic purity HPLC data were obtained on an Agilent 1100 system and peak identity was confirmed by LCMS on an Agilent 1260 HPLC with an Agilent 6130 single quadrupole mass spectroscopic detector using ESI. Each of these latter two systems was coupled to a Dionex Corona Ultra RS CAD as required.

### Solubilization of Compounds for Biological Studies:

Solubilization of α-Gal-Cer and α-Gal-Cer-peptide conjugates was achieved by lyophilizing the samples in the presence of aqueous sucrose, L-histidine and Tween 20 as previously described for the solubilization of α-Gal-Cer (Giaccone G, 2002). Typically, all compounds were reconstituted in water then further diluted in PBS for i.v administration.

### Cathepsin B Assay Reaction

A stock solution of phytosphingosine (190 μM) in DMSO was pre-mixed with ammonium acetate buffer (50 mM, pH 5.3) containing EDTA (2.5 mM) and dithiothreitol (2.5 mM) to a final phytosphingosine concentration of 6.3 uM. The substrate conjugate (190 μM in DMSO) was added to the pre-mixed buffer solution to give a final substrate concentration of 12.7 μM. Cathepsin B from human liver (Sigma) dissolved in ammonium acetate buffer (50 mM, pH 5.3, EDTA (2.5 mM), dithiothreitol (2.5 mM)) was added to the reaction mixture to give a final cathepsin B concentration of 2.9 units/mL. For the control reaction (without enzyme) the same volume of buffer was added. The reaction mixtures were then incubated at 37° C. Aliquots of 10 uL were taken from the reactions and analysed by LCMS at 1, 4 and 24 hours after start of reaction.

### CD8⁺ T Cell Transfer

Naïve PbT-I CD8⁺ T cells were isolated by negative selection from the lymph nodes and/or spleen as previously described (Fernandez-Ruiz D, 2016). Briefly, tissues were disrupted by passing through 70 μm cell strainers and red cells lysed. Single cell suspensions were labeled with a cocktail of rat monoclonal antibodies specific for mouse CD4, MHC Class II, macrophages and neutrophils prior to incubating with BioMag goat anti-rat IgG beads (Qiagen, Chadstone, VIC, Australia) and separated using a magnet. Enriched naïve CD8⁺ T cells were counted and their purity analyzed by staining with anti-CD8α and anti-Vα_{8.3} TCR antibodies. Cell counts were adjusted to 2.5×10⁵/mL in PBS and mice were injected with 200 μL i.v. OT-I cells were isolated from pooled lymph nodes. A portion of OT-I cells were stained with anti-CD8α, anti-Vα₂, anti-CD44 and anti-CD62L antibodies to determine the proportion of naïve (CD8⁺CD44^{lo}CD62L^{hi}) CD8⁺ T cells. 10⁴ naïve CD8⁺ OT-I T cells were then injected i.v. into each mouse across all treatment groups. Mice were injected with naïve OT-I or PbT-I cells i.v one day prior to vaccination with 600 pfu of recombinant PR8-OVA (H1N1) influenza virus, 0.135 nmoles of αGalCer, or 0.135 nmoles conjugate-vaccines i.v. Naive PbT-I cells were primed in B6 mice by i.v injection of 5 nmol of CpG 2006-21798 (Fernandez-Ruiz D, 2016) (Krieg, 2006) and 8 μg of anti-Clec9A antibody genetically fused to LSNYVDFNLLLERD (SEQ ID NO: 14) (Fernandez-Ruiz D, 2016) (Caminschi I, 2008). In some experiments, mice receiving anti-Clec9a were additionally treated with 2.5×10⁹ copies of AAV-NVY (SEQ ID NO: 15) (Fernandez-Ruiz D, 2016).

Lymphocyte Isolation from Organs

Tissues were harvested from mice at different time points after immunization. For spleen cell preparations, the organ was passed through 70 μm mesh and red blood cells were lysed. Liver cell suspensions were passed through 70 μm mesh and resuspended in 35% isotonic Percoll (Sigma). Cells were then centrifuged at 500 g for 20 min at RT, the pellet harvested and then red cells lysed before further analysis.

### Flow Cytometry

Lymphocytes were stained with monoclonal antibodies for: CD49a (Ha31/8), NK1.1 (PK136) from BD (North Ryde, NSW, Australia), CD8a (53-6.7), KLRG1 (2F1), Ly5.1 (A20), Ly5.1 (104), CD8a (53-6.7), CD44 (IM7), Ly6C (HK1.4), TCRβ (H57-597), CXCR6 (SA051D1), CXCR3 (CXCR3-173), CX3CR1 (SA011F11), from Biolegend (Australian Biosearch, Karrinyup, WA. Australia), and CD62L (MEL-14), CD101 (Moushi101) and CD69 (H1.2F3), from eBioscience (Jomar Life Research, Scoresby, VIC, Australia). Dead cells were excluded by propidium iodide staining or far red live/dead fixable dye (ThermoFisher). In some experiments cells were stained with an α-GalCer (PBS-44—a gift from Prof. Paul Savage, Brigham-Young University, UT, USA)-loaded CD1d tetramer produced in-house at 4° C. for 30 min, washed, and further antibody staining was conducted for 10 min at 4° C. Antibodies used were for: CD3 (17A2), CD45R/B220 (RA3-6B2), NK1.1 (PK136), CD69 (H1.2F3), all from BioLegend (CA, USA). The viability dye used was DAPI (Invitrogen, NZ). Single-color positive control samples were used to adjust compensation and cells were analyzed by flow cytometry on a LSR Fortessa (BD Biosciences), or LSRII SORP using Flowjo software (Tree Star Inc.).

### Serum ALT Measurements

Measurement of serum ALT levels was performed with a modular analyzer (Roche/Hitachi Modular P800, Roche Diagnostics, Indianapolis, Ind.) by Gribbles Veterinary Clinic (Hamilton, New Zealand) according to a standard operating procedure approved by International Accreditation New Zealand.

### Statistical Analysis

Figures were generated using GraphPad Prism 7. Data are shown as mean values±S.E.M as indicated in the figure legends. Data was log transformed, assessed for normality then a one way ANOVA with Tukey's multiple comparison test was performed. To compare survival after challenge, groups were compared using Fisher's exact test. The statistical tests performed on the data are indicated in the figure legends and results, along with sample size indicating the number of animals used. P-values <0.05 (*), <0.01 (**), 0.001 (***) or 0.0001 (****) were considered statistically significant.

### Labelling Convention for SEQ ID NO:

In the following examples, and elsewhere in the specification, amino acid or nucleic acid sequences present in the conjugates described herein are indicated by SEQ ID NO: X-SEQ ID NO: Y. This labeling convention does not indicate a sequence range, but rather discrete regions of amino acid sequence residues that are joined together in the conjugates; e.g. SEQ ID NO: 1 “joined to” SEQ ID NO: 4 (SEQ ID NO: 1-SEQ ID NO: 4).

### Example 1—Synthesis of Peptides for Conjugation 5-Azidopentanoyl-FFRK-AAAHSLSNVYDFNLLLERD (SEQ ID NO: 1—SEQ ID NO: 3)

5-Azidopentanoyl-FFRK-AAAHSLSNVYDFNLLLERD (SEQ ID NO: 1-SEQ ID NO: 3) was synthesized by Fmoc SPPS on a 4-(4-hydroxymethyl-3-methoxyphenoxy)butyric acid (HMPB) ChemMatrix® resin preloaded with Fmoc-L-aspartic acid 4-tert-butyl ester (Fmoc-Asp(tBu)) as the first amino acid. α-Amino acids with the following side chain protecting groups were used: Arg(Pbf), Lys(Boc), Ser(tBu), Asn(Trt), His(Trt), Tyr(tBu), Asp(tBu), Glu(tBu) and the peptide was synthesized using a Biotage® Initiator+ Alstra™ microwave peptide synthesizer on a 0.1 mmol scale. The resin was swelled in DMF (20 min, 70° C., 50 W) followed by synthesis reaction cycles consisting of: Fmoc deprotection with 20% piperidine in DMF (3 min and then 10 min, rt); and amino acid coupling (5 min, 75° C., 50 W) employing 5 equivalents of the protected amino acid in DMF (0.5 M) activated by 5 equivalents of diisopropylcarbodiimide and Oxyma® (both 0.5 M in DMF). Fmoc-Arg(Pbf) and Fmoc-His(Trt) were coupled for 60 min at rt. 5-Azidopentanoic acid (0.5 M in DMF) was incorporated at the N-terminus using standard amino acid coupling conditions following the final Fmoc deprotection.

The resin was washed with CH₂Cl₂ and dried under vacuum. The subsequent cleavage from the resin was achieved by incubating the resin in 10 mL of 88:5:5:2 TFA/water/phenol/i-Pr₃SiH at 0° C. for 10 min. The resin was filtered, rinsed with a further 10 mL of the cleavage solution and left to stand at room temperature for 2 h. Crude peptide was precipitated and triturated with cold diethyl ether, isolated (centrifugation), and lyophilized from 95:5:0.2 water/MeCN/TFA. The peptide was dissolved in DMSO (40 mg/mL) and purified on an Agilent 1260 Infinity HPLC system by successive injections of −30 mg onto a Phenomenex Luna C18(2) 4.6 μm, 250×21.2 mm column, using a linear gradient from 36% MeCN/water (0.1% TFA) to 50% MeCN/water (0.1% TFA) over 10 min (flow=16 mL/min, T=40° C.). Fractions containing the desired peptide at sufficient purity were pooled and lyophilized. The purified peptide showed a main peak for the target peptide with a retention time of 9.27 min and a minor (1.6%) impurity at 9.14 min. The mass signal at m/z 951.4 (calculated 951.2 for [M+3H]³⁺) confirmed the identity of the major product.

### Aminooxyacetyl-FFRK-NVFDFNNL (SEQ ID NO: 1-SEQ ID NO: 5)

Aminooxyacetyl-FFRKNVFDFNNL (SEQ ID NO: 1-SEQ ID NO: 5) was synthesized by Fmoc SPPS, as described for 5-Azidopentanoyl-FFRK-AAAHSLSNVYDFNLLLERD (SEQ ID NO: 1-SEQ ID NO: 3), on a 4-(4-hydroxymethyl-3-methoxyphenoxy)butyric acid (HMPB) ChemMatrix® resin preloaded with Fmoc-L-aspartic acid 4-tert-butyl ester (Fmoc-Asp(tBu)) as the first amino acid. α-Amino acids with the following side chain protecting groups were used: Arg(Pbf), Lys(Boc), Asn(Trt), Asp(tBu) and the peptide was synthesized on a 0.1 mmol scale. Fmoc-Arg(Pbf) was coupled for 60 min at rt. Aminooxyacetic acid (3 equivalents, 0.06 M in DMF) was incorporated at the N-terminus by stirring with 7 equivalents of 2,4,6-trimethylpyridine (20 minutes, rt) following the final Fmoc deprotection. The resin was washed with 1:1 CH₂Cl₂/isopropanol and dried under vacuum. The subsequent cleavage from the resin was achieved by incubating the resin in 5 mL of 88:5:5:2 TFA/water/phenol/i-Pr₃SiH and 180 mg aminooxyacetic acid hemihydrochloride at 0° C. for 10 min. The resin was filtered, rinsed with a further 5 mL of the cleavage solution and left to stand at room temperature for 2 h. Crude peptide was precipitated, triturated and lyophilised as described for 5-Azidopentanoyl-FFRK-AAAHSLSNVYDFNLLLERD (SEQ ID NO: 1-SEQ ID NO: 3). The peptide was dissolved in DMSO (40 mg/mL), diluted to 1.8 mg/mL with water (0.2% TFA) and purified on an Agilent 1260 Infinity HPLC system by loading onto a Phenomenex Gemini 5 um C18 110A, 250×10 mm column, using a linear gradient from 21% MeCN/water (0.1% TFA) to 31% MeCN/water (0.1% TFA) over 100 min (flow=5 mL/min, T=40° C.). Fractions containing the desired peptide at sufficient purity were pooled and lyophilized. The purified peptide showed a main peak for the target peptide (96%) with a mass signal at m/z 817.4 (calculated 817.9 for [M+2H]2+) confirmed the identity of the product.

### Aminooxyacetyl-FFRK-AAASTNVFDFNNLS (SEQ ID NO: 1-SEQ ID NO: 5)

Aminooxyacetyl-FFRK-AAASTNVFDFNNLS (SEQ ID NO: 1-SEQ ID NO: 5) was synthesized by Fmoc SPPS, as described for 5-Azidopentanoyl-FFRK-AAAHSLSNVYDFNLLLERD (SEQ ID NO: 1-SEQ ID NO: 3), on a 4-(4-hydroxymethyl-3-methoxyphenoxy)butyric acid (HMPB) ChemMatrix® resin preloaded with Fmoc-O-tert-butyl-L-serine (Fmoc-Ser(tBu)) as the first amino acid. α-Amino acids with the following side chain protecting groups were used: Arg(Pbf), Lys(Boc), Ser(tBu), Thr(tBu), Asn(Trt), Asp(tBu) and the peptide was synthesized on a 0.1 mmol scale. Fmoc-Arg(Pbf) was coupled for 60 min at rt. Aminooxyacetic acid (3 equivalents, 0.06 M in DMF) was incorporated at the N-terminus by stirring with 7 equivalents of 2,4,6-trimethylpyridine (20 minutes, rt) following the final Fmoc deprotection. The resin was washed with 1:1 CH₂Cl₂/isopropanol and dried under vacuum. The subsequent cleavage from the resin was achieved by incubating the resin in 8 mL of 88:5:5:2 TFA/water/phenol/i-Pr₃SiH and 180 mg aminooxyacetic acid hemihydrochloride at 0° C. for 10 min. The resin was filtered, rinsed with a further 8 mL of the cleavage solution and left to stand at room temperature for 2 h. Crude peptide was precipitated, triturated and lyophilised as described for 5-Azidopentanoyl-FFRK-AAAHSLSNVYDFNLLLERD (SEQ ID NO: 1-SEQ ID NO: 3). The peptide was dissolved in DMSO (30 mg/mL) and purified on an Agilent 1260 Infinity HPLC system by successive injections of ˜10 mg onto a Phenomenex Luna C18(2) 4.6 μm, 250×21.2 mm column, using a linear gradient from 40% MeCN/water (0.1% TFA) to 55% MeCN/water (0.1% TFA) over 10 min (flow=16 mL/min, T=40° C.). Fractions containing the desired peptide at sufficient purity were pooled and lyophilized. The purified peptide showed a main peak for the target peptide (93%) with a mass signal at m/z 1061.9 (calculated 1062.2 for [M+2H]2+) confirmed the identity of the major product.

5-Azidopentanoyl-AAAHSLSNVYDFNLLLERD (SEQ ID NO: 3), 5-azidopentanoyl-FFRK-NVYDFNLL (SEQ ID NO: 1-SEQ ID NO: 2), aminooxyacetyl-FFRK-NVFDFNLL (SEQ ID NO: 1-SEQ ID NO: 4), and aminooxyacetyl-FFRK-EIYIFTNI (SEQ ID NO: 13) were obtained from commercial manufacturer Peptides and Elephants.

### Example 2: Synthesis of Glycolipid-Linker for Oxime Conjugation

### 4-(N-((9H-Fluoren-9-yl)methoxycarbonyl)-L-valinyl-L-citrullinamido)benzyl 4-nitrophenyl carbonate (Fmoc-VCPAB-pNP)

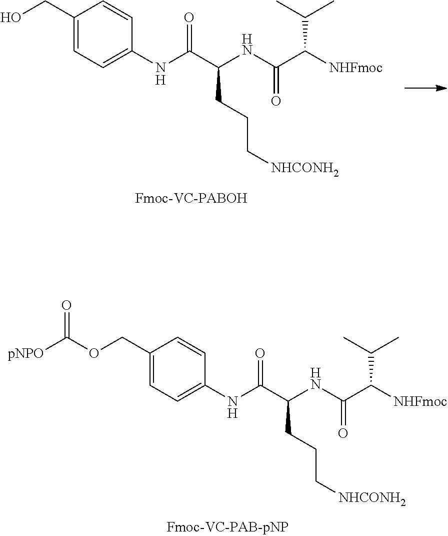

To a mixture of alcohol Fmoc-VC-PABOH (Dubowchik, 2002) (400 mg, 0.665 mmol) in DMF (6.0 mL) was added bis(4-nitrophenyl) carbonate (255 mg, 0.796 mmol) and i-Pr₂NEt (0.13 mL, 0.75 mmol). After stirring under Ar at rt for 18 h, the solvent was co-evaporated several times with toluene in a rotary evaporator. Purification by flash chromatography on silica gel (gradient elution, MeOH/CHCl₃=0:100 to 8:92) gave the title compound as a pale yellow solid (380 mg, 75%). ¹H NMR (500 MHz, d₆-DMSO) δ 0.85 (d, J=6.7 Hz, 3H), 0.88 (d, J=6.7 Hz, 3H), 1.33-1.49 (m, 2H), 1.56-1.64 (m, 1H), 1.67-1.74 (m, 1H), 1.95-2.02 (m, 1H), 2.91-2.97 (m, 1H), 2.99-3.06 (m, 1H), 3.92 (dd, J=7.2, 8.7 Hz, 1H), 4.20-4.26 (m, 2H), 4.28-4.33 (m, 1H), 4.39-4.43 (m, 1H), 5.24 (s, 2H), 5.39 (br s, 2H), 5.98 (br t, J=5.7 Hz, 1H), 7.30-7.33 (m, 2H), 7.36-7.42 (m, 5H), 7.54-7.57 (m, 2H), 7.63 (d, J=8.4 Hz, 2H), 7.70-7.74 (m, 2H), 7.87 (d, J=7.4 Hz, 2H), 8.10 (d, J=7.4 Hz, 1H), 8.29-8.32 (m, 2H), 10.10 (s, 1H); ¹³C NMR (126 MHz, d₆-DMSO) δ 18.3, 19.2, 26.8, 29.4, 30.5, 38.7, 46.8, 53.2, 60.2, 65.8, 70.3, 119.2, 120.1, 122.7, 125.4, 125.5, 127.2, 127.7, 129.46, 129.54, 139.4, 140.8, 143.8, 143.9, 145.3, 152.0, 155.4, 156.2, 159.1, 170.8, 171.4; HRMS-ESI: m/z calcd for C₄₀H₄₃N₆O₁₀ [M+H]+ 767.3041, found 767.3070.

### (2S,3S,4R)-2-(N-((9H-Fluoren-9-yl)methoxycarbonyl)-L-valinyl-L-citrullinyl-4-aminobenzyloxycarbonylamino)-1-(δ-D-galactopyranosyloxy)-3-hydroxy-octadecan-4-yl hexacosanoate (MaGC-PAB-CV-Fmoc)

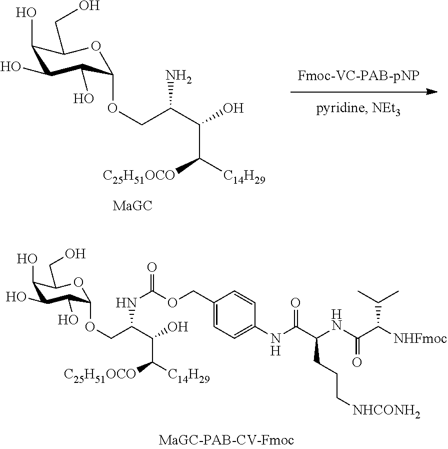

To a mixture of amine MaGC (Anderson, 2014)((73 mg, 0.085 mmol) and carbonate Fmoc-VC-PAB-pNP (93 mg, 0.12 mmol) in anhydrous pyridine (1.5 mL) at rt under Ar was added Et₃N (16 μL, 12 mg, 0.11 mmol). After stirring for 18 h, the mixture was concentrated to dryness and purified by column chromatography on silica gel (MeOH/CHCl₃=0:100 to 20:80) to afford the title compound as a white solid (66 mg, 52%). ¹H NMR (500 MHz, 2:3 CDCl₃/CD₃OD) δ 0.87-0.90 (m, 6H), 0.95-0.98 (m, 6H), 1.24-1.37 (m, 68H), 1.51-1.78 (m, 7H), 1.89-1.96 (m, 1H), 2.07-2.13 (m, 1H), 2.32-2.42 (m, 2H), 3.07-3.13 (m, 1H), 3.20-3.25 (m, 1H), 3.66-3.81 (m, 8H), 3.84-3.87 (m, 2H), 3.99 (d, J=6.7 Hz, 1H), 4.24 (t, J=6.9 Hz, 1H), 4.37 (dd, J=6.9, 10.5 Hz, 1H), 4.45 (dd, J=6.9, 10.5 Hz, 1H), 4.54 (dd, J=5.2, 8.6 Hz, 1H), 4.84 (d, J=3.7 Hz, 1H), 4.97-5.03 (m, 2H), 5.06-5.10 (m, 1H), 7.30-7.33 (m, 4H), 7.38-7.41 (m, 2H), 7.58 (d, J=8.1 Hz, 2H), 7.63-7.65 (m, 2H), 7.78 (d, J=7.6 Hz, 2H); ¹³C NMR (126 MHz, 2:1 CDCl₃/CD₃OD) δ 14.3, 18.2, 19.4, 23.0, 25.5, 25.7, 26.7, 29.2, 29.6, 29.27, 29.74, 29.8, 29.93, 29.95, 29.98, 30.02, 30.05, 30.08, 30.10, 31.4, 32.3, 35.0, 39.4, 47.6, 52.7, 53.8, 61.2, 62.3, 66.8, 67.4, 68.4, 69.4, 70.2, 70.7, 71.0, 72.3, 75.1, 100.4, 120.3, 120.5, 125.40, 125.44, 127.5, 128.2, 129.1, 133.0, 138.2, 141.7, 144.2, 144.3, 157.1, 157.6, 161.1, 171.1, 173.2, 175.0; HRMS-ESI m/z calcd for C₈₄H₁₃₇N₆O₁₆ [M+H]⁺ 1486.0091, found 1486.0099.

### (2S,3S,4R)-2-(L-Valinyl-L-citrullinyl-4-aminobenzyloxycarbonylamino)-1-(δ-D-galactopyranosyloxy)-3-hydroxy-octadecan-4-yl hexacosanoate (MaGC-PAB-CV-NH₂)

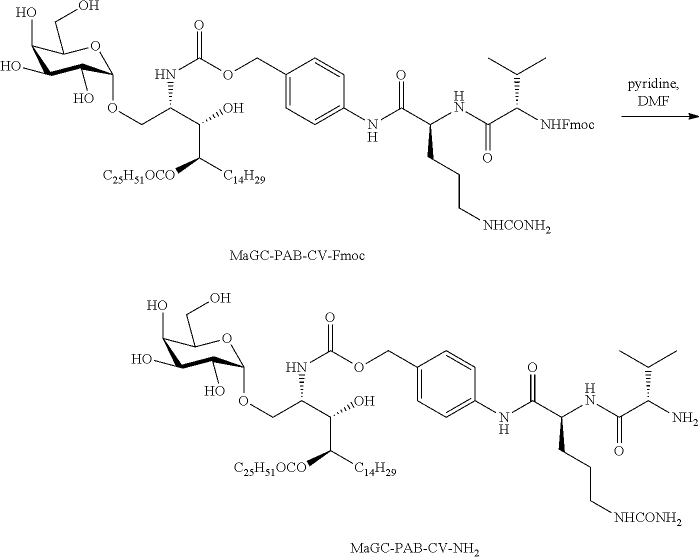

To an ice-cooled solution of MaGC-PAB-CV-Fmoc (66 mg, 0.044 mmol) in anhydrous DMF (2 mL) under Ar was added piperidine (0.20 mL, 2.0 mmol). After 5 min the mixture was warmed to rt and stirred for a further 30 min, before concentrating under high vacuum. Purification by flash chromatography on silica gel (MeOH/CHCl₃=0:100 to 60:40) gave the title compound as a white solid (45 mg, 81%). ¹H NMR (500 MHz, 2:1 CDCl₃/CD₃OD) δ 0.87-0.91 (m, 9H), 1.00 (d, J=6.9 Hz, 3H), 1.23-1.35 (m, 68H), 1.49-1.77 (m, 7H), 1.87-1.94 (m, 1H), 2.07-2.13 (m, 1H), 2.32-2.39 (m, 2H), 3.10-3.16 (m, 1H), 3.21 (d, J=4.9 Hz, 1H), 3.24-3.29 (m, 1H), 3.65-3.80 (m, 8H), 3.85-3.87 (m, 2H), 4.57 (dd, J=5.3, 8.5 Hz, 1H), 4.85 (d, J=3.7 Hz, 1H), 4.92-4.99 (m, 2H), 5.10-5.15 (m, 1H), 7.33 (d, J=8.3 Hz, 2H), 7.56 (d, J=8.3 Hz, 2H); ¹³C NMR (75 MHz, 3:1 CDCl₃/CD₃OD) δ 14.1, 16.8, 19.5, 22.8, 25.2, 25.5, 26.4, 29.0, 29.4, 29.5, 29.6, 29.7, 29.8, 29.9, 30.0, 31.9, 32.1, 34.8, 39.2, 52.3, 53.1, 60.4, 62.1, 66.6, 68.2, 69.2, 70.0, 70.5, 70.6, 72.2, 74.9, 100.1, 120.3, 128.9, 132.9, 138.0, 156.8, 160.8, 171.1, 174.8, 175.7; HRMS-ESI m/z calcd for C₆₉H₁₂₂N₆O₁₄ [M+H]⁺ 1263.9410, found 1263.9419.

### (2S,3S,4R)-2-(N-(8-Oxononanoyl)-L-valinyl-L-citrullinyl-4-aminobenzyloxycarbonylamino)-1-(δ-D-galactopyranosyloxy)-3-hydroxy-octadecan-4-yl hexacosanoate (MaGC-PAB-CV-Non)

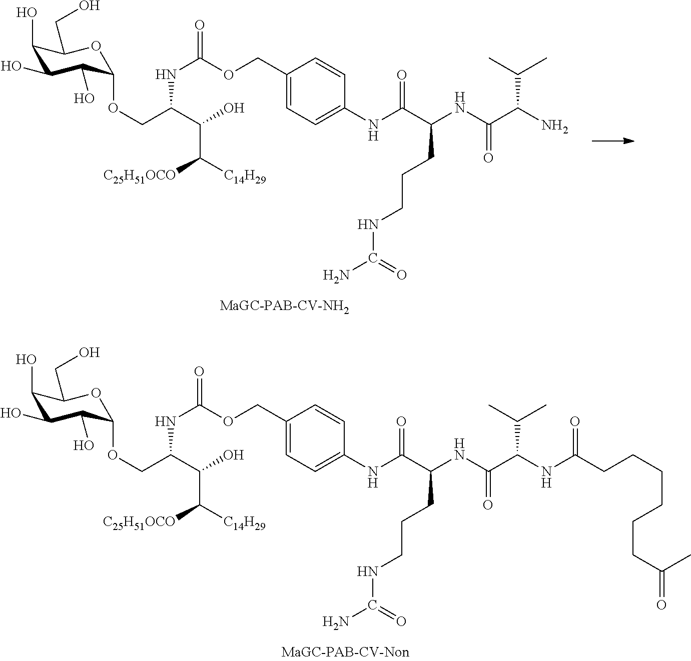

A DMF-solution (250 uL) containing 8-oxononanoic acid (2.2 mg, 12 μmol), i-Pr₂NEt (2.5 μL, 14 μmol) and 2-(1H-benzotriazol-1-yl)-1,1,3,3-tetramethyluronium hexafluorophosphate (HBTU) (2.6 mg, 6.8 μmol) was added to amine MaGC-PAB-CV-NH₂ (6.9 mg, 5.5 μmol) and the mixture was stirred at rt for 17 h. After concentrating under vacuum, the residue was purified by flash chromatography on silica gel (MeOH/CH₂Cl₂=8:92 to 20:80) and subsequently triturated with water to give the title compound as a white solid (6.6 mg, 85%). ¹H NMR (500 MHz, 2:1 CDCl₃/CD₃OD) δ 0.87-0.90 (m, 6H), 0.95-0.97 (m, 6H), 1.11-1.43 (m, 72H), 1.50-1.77 (m, 11H), 1.87-1.94 (m, 1H), 2.02-2.11 (m, 1H), 2.15 (s, 3H), 2.22-2.31 (m, 2H), 2.32-2.41 (m, 2H), 2.46 (t, J=7.3 Hz, 2H), 3.09-3.14 (m, 1H), 3.21-3.26 (m, 1H), 3.64-3.83 (m, 8H), 3.85-3.92 (m, 2H), 4.18 (d, J=7.3 Hz, 1H), 4.54 (dd, J=5.1, 8.5 Hz, 1H), 4.85 (d, J=3.7 Hz, 1H), 4.93-5.02 (m, 1H), 5.13 (d, J=12.2 Hz, 1H), 7.32 (d, J=8.2 Hz, 2H), 7.57 (d, J=8.2 Hz, 2H); ¹³C NMR (126 MHz, 2:1 CDCl₃/CD₃OD) δ 14.21, 14.22, 18.5, 19.4, 23.0, 23.9, 25.3, 25.4, 25.7, 25.9, 26.7, 29.1, 29.3, 29.6, 29.69, 29.71, 29.8, 29.89, 29.91, 29.95, 29.98, 30.01, 30.04, 30.05, 30.06, 31.0, 32.3, 35.0, 36.4, 43.9, 52.6, 53.7, 59.4, 62.3, 66.8, 68.4, 69.4, 70.2, 70.7, 71.0, 72.3, 75.1, 100.4, 120.5, 129.1, 133.0, 157.1, 161.1, 171.0, 172.9, 175.0, 175.2, 211.4; HRMS-ESI m/z calcd for C₂₈H₁₄₀N₆NaO₁₆ [M+Na]⁺1440.0218, found 1440.0214.

### Example 3: Synthesis of Glycolipid-Peptide Conjugates

### SPAAC Conjugate Vaccines

MaGC-PAB-CV-cyclooctyne and conjugate compound V.S.FFRK.OVA_{LP} (C₂₆) were synthesized as previously described (Anderson R J, 2017)

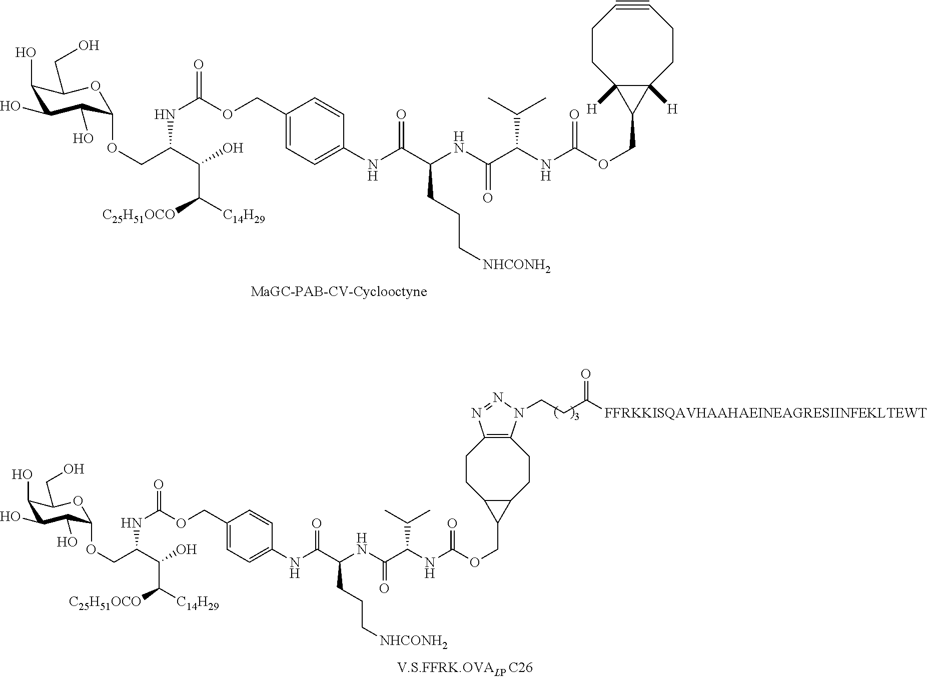

### Conjugate V.S.FFRK.NVY_{SP}

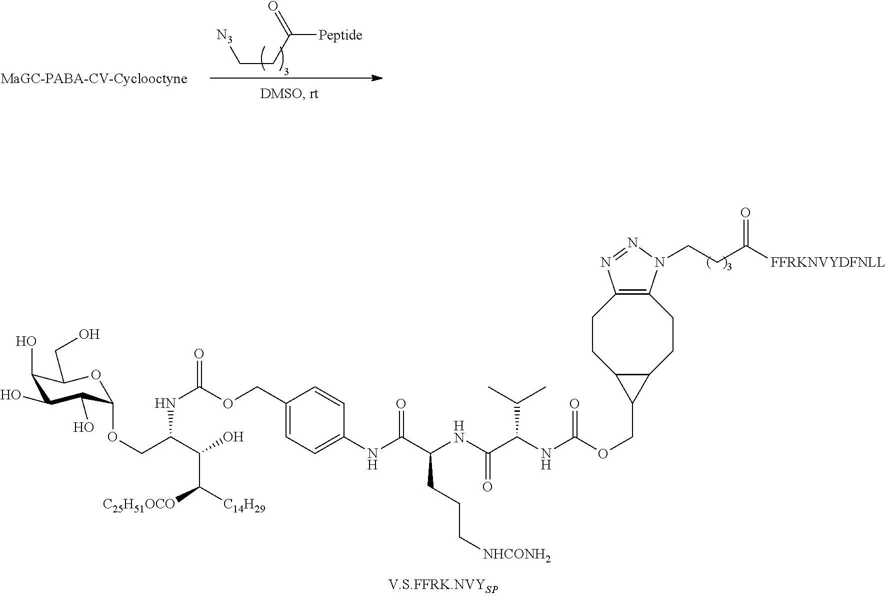

A solution of 5-azidopentanoyl-FFRK-NVYDFNLL (SEQ ID NO: 1-SEQ ID NO: 2) (1.6 mg, 0.96 μmol) and MaGC-PABA-CV-cyclooctyne (1.0 mg, 0.69 μmol) in DMSO (100 μL) was kept at rt for 2 days. The product solution was purified by semi-preparative HPLC (Phenomenex Luna C18(1), 4.6 μm, 250×10 mm, 40° C., Mobile phase A=100:0.05 water/TFA; Mobile phase B=100:0.05 MeOH/TFA; Gradient program: T0=80% B, T12=100% B, T14=100% B, T14.5=80% B, T16=80% B; Flow: T0=3 mL/min, T1=3 mL/min, T12=4.5 mL/min, T14=4.5 mL/min, T16=3 mL/min) to give V.S.FFRK.NVY_{SP} as a white powder (1.8 mg, 82%). HRMS-ESI m/z calcd for C₁₆₂H₂₅₇N₂₇O₃₅ [M+2H]²⁺1570.4580, found 1570.4591.

### Conjugate V.S.NVY_{LP}

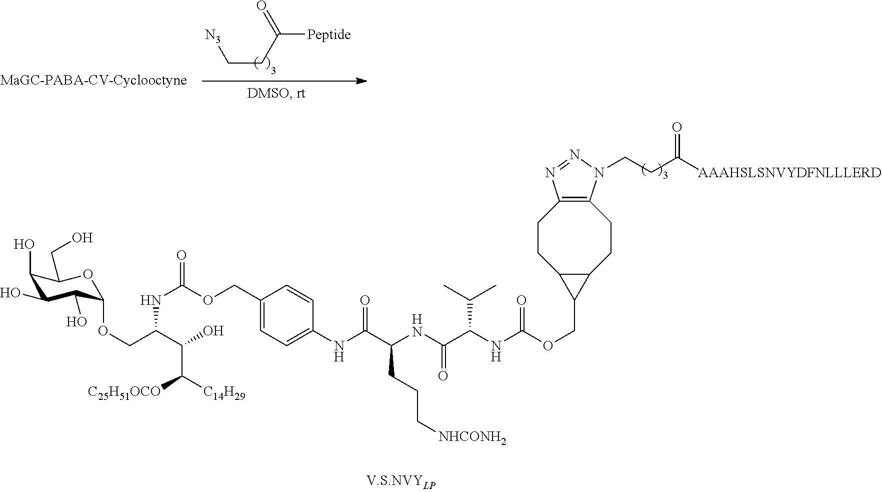

A solution of 5-azidopentanoyl-AAAHSLSNVYDFNLLLERD (SEQ ID NO: 3) (2.2 mg, 0.97 μmop and MaGC-PABA-CV-cyclooctyne (1.0 mg, 0.69 μmop in DMSO (100 μL) was kept at rt for 2 days. The product solution was purified as described above for V.S.FFRK.NVY_{SP} to give V.S.NVY_{LP} as a white powder (1.9 mg, 73%). HRMS-ESI m/z calcd for C₁₈₀H₂₉₃N₃₅O₄₈ [M+2H]²⁺ 1856.5781, found 1856.5786.

### Conjugate V.S.FFRK.NVY_{LP}

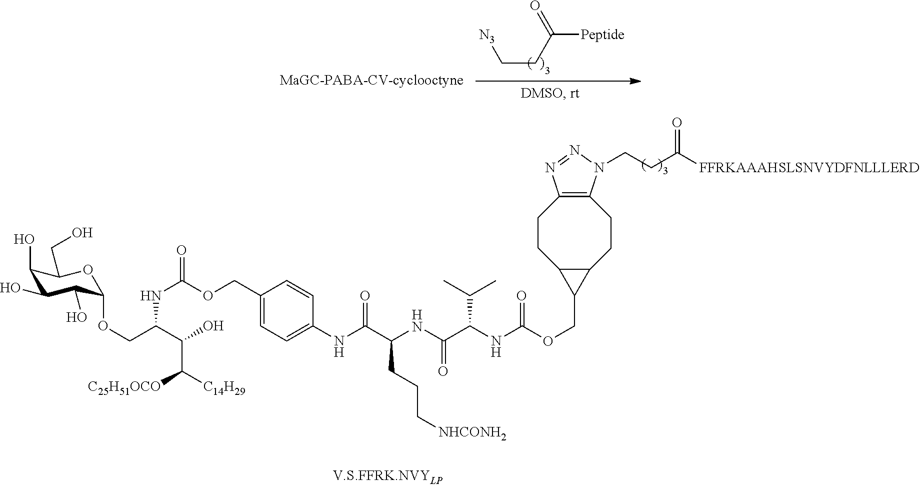

A solution of 5-azidopentanoyl-FFRK-AAAHSLSNVYDFNLLLERD (SEQ ID NO: 1-SEQ ID NO: 3) (8.4 mg, 2.9 μmop and MaGC-PABA-CV-cyclooctyne (2.9 mg, 2.0 μmop in DMF (300 μL) was stood at rt for 1 day. The crude product, V.S.FFRK.NVY_{LP}, solution was diluted with further DMF (300 μL) and used as is for formulation. A volume of 73 μL (theoretically 1 mg of product) was diluted with DMSO (127 μL) to give a 5 mg/mL solution, which was formulated as described in the Methods Section. HRMS-ESI m/z calcd for C₂₁₀H₃₃₆N₄₃O₅₂ [M+3H]³⁺ 1430.8318, found 1430.8323.

### Oxime Conjugate Vaccines

### Conjugate V.Ox.FFRK.NVY_{SP}

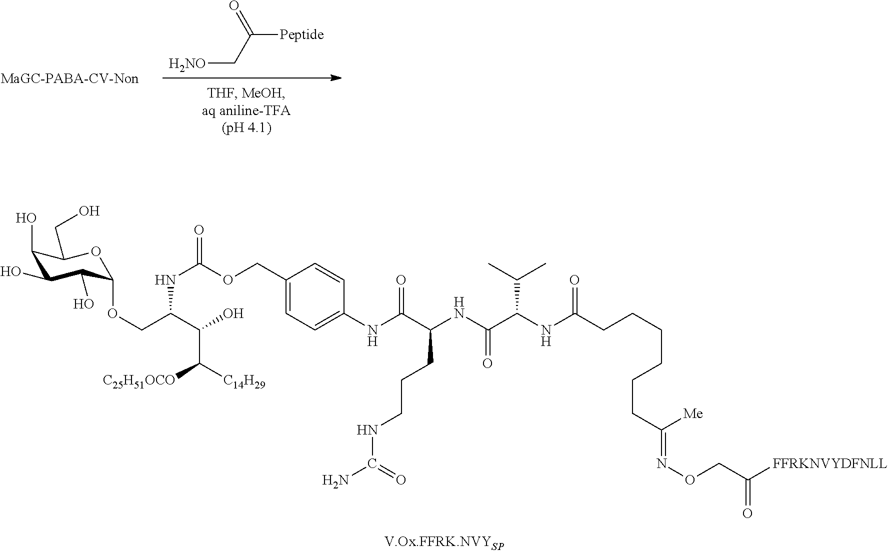

Aniline buffer (pH=4.1, 300 mM) was prepared by mixing freshly distilled aniline (5.5 mL) and TFA (3.9 mL) in MilliQ water, and making up to a total volume of 200 mL. THF was distilled from 2,4-dinitrophenylhydrazine. A mixture of peptide aminooxyacetyl-FFRK-NVYDFNLL (SEQ ID NO: 1-SEQ ID NO: 2) (3.9 mg, 2.4 μmol) and ketone MaGC-PAB-CV-Non (2.0 mg, 1.4 μmol) was heated in 4:2:3 THF/MeOH/aniline buffer (300 μL) at 50° C. for 16 h. The product mixture was purified by preparative HPLC [Phenomenex Luna C18(2), 5 μm, 250×21.2 mm, 30° C., 17 mL/min; Mobile phase A=30:70:0.05 water/MeOH/TFA; Mobile phase B=100:0.05 MeOH/TFA; 0-13 min: 100% A to 100% B; 13-15 min: 100% B; 15-16 min: 100% B to 100% A; 16-18 min: 100% A1 to give the title compound V.Ox.FFRK.NVY_{SP} as a white solid (3.3 mg, 77%). HRMS-ESI m/z calcd for C₁₅₇H₂₅₂N₂₅O₃₅ [M+2H]²⁺ 1524.4388, found 1524.4380.

### Conjugate V.Ox.FFRK.NVF_{SP}

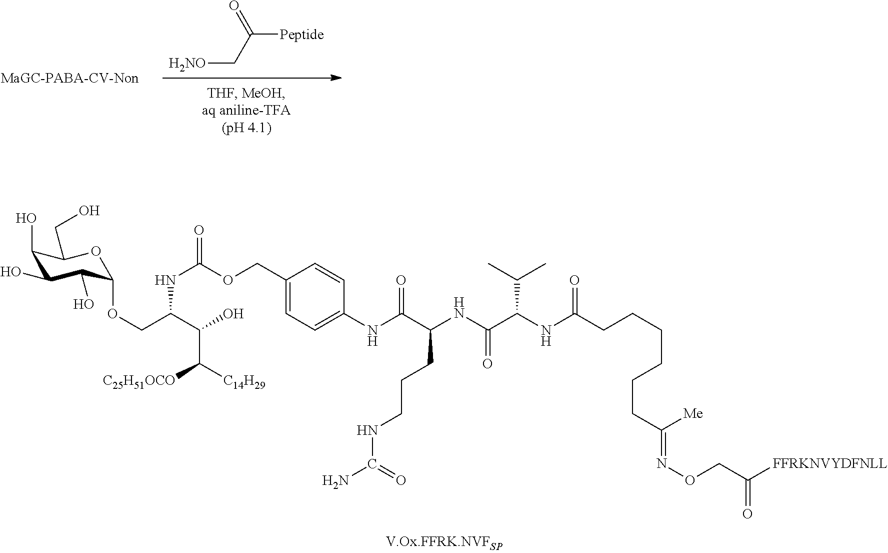

A mixture of peptide aminooxyacetyl-FFRKNVFDFNNL (SEQ ID NO: 1-SEQ ID NO: 4) (4.4 mg, 2.7 μmol) and ketone MaGC-PAB-CV-Non (2.1 mg, 1.5 μmol) was heated in 4:2:3 THF/MeOH/aniline buffer (300 μL, preparation as described above for V.Ox.FFRK.NVY_{SP}) at 50° C. for 16 h. The product solution was purified as described above for V.Ox.FFRK.NVY_{SP} to give V.S.NVF_{SP} as a white powder (2.1 mg, 47%). HRMS-ESI m/z calcd for C₁₅₅H₂₄₆N₂₆O₃₅ [M+2H]²⁺ 1516.9252, found 1516.9213.

### Conjugate V.Ox.FFRK.NVF_{LP}

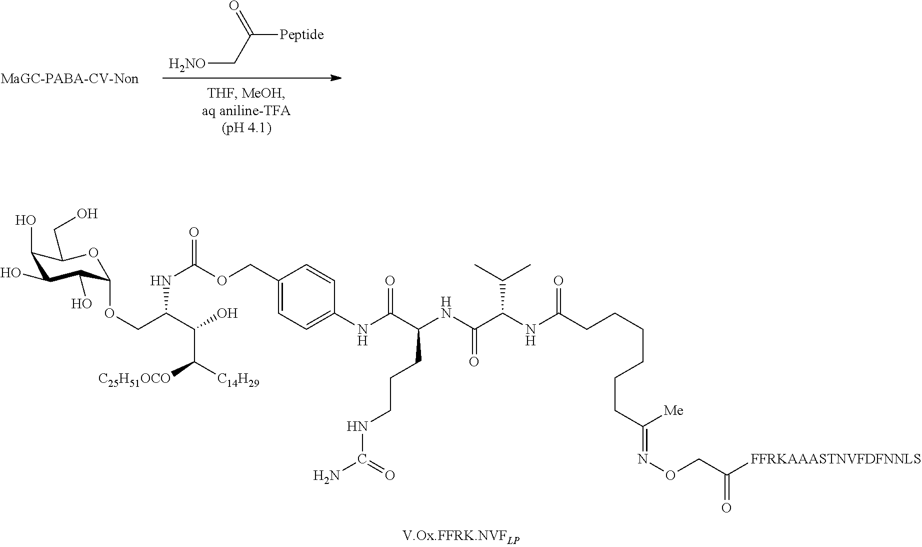

A mixture of peptide aminooxyacetyl-FFRKAAASTNVFDFNNLS (SEQ ID NO: 1—SEQ ID NO: 5) (4.3 mg, 2.0 μmol) and ketone MaGC-PAB-CV-Non (2.0 mg, 1.4 μmol) was heated in 4:2:3 THF/MeOH/aniline buffer (300 μL, preparation as described above for V.Ox.FFRK.NVY_{SP}) at 50° C. for 16 h. The product solution was purified as described above for V.Ox.FFRK.NVY_{SP} to give V.S.NVF_{LP} as a white powder (2.2 mg, 46%). HRMS-ESI m/z calcd for C₁₇₄H₂₇₈N₃₂O₄₄ [M+3H]3+ 1174.3578, found 1174.3564.

### Conjugate V.Ox.FFRK.EIY_{SP}

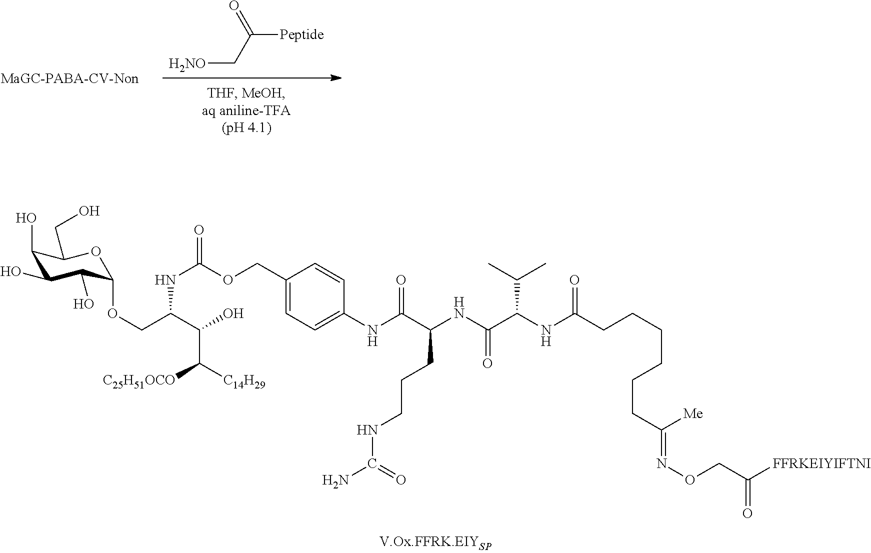

A mixture of peptide aminooxyacetyl-FFRK-EIYIFTNI (SEQ ID NO: 1-SEQ ID NO: 13) (3.9 mg, 2.3 μmol) and ketone MaGC-PAB-CV-Non (2.0 mg, 1.4 μmol) was heated in 4:2:3 THF/MeOH/aniline buffer (300 μL, preparation as described above for V₂₆Ox.FFRK.NVY_{SP}) at 50° C. for 16 h. The product solution was purified as described above for V₂₆Ox.FFRK.NVY_{SP} to give V.S.EIY_{SP} as a white powder (3.6 mg, 84%). HRMS-ESI m/z calcd for C₁₅₉H₂₅₆N₂₄O₃₅ [M+2H]²⁺ 1531.9573, found 1531.9589.

### Example 4: Synthesis of Conjugates with Varying Length of Fatty Acid (R1)

Prodrug compounds contain fatty acids of different lengths were chemically synthesized (Scheme 1) using the General methods B-E described below.

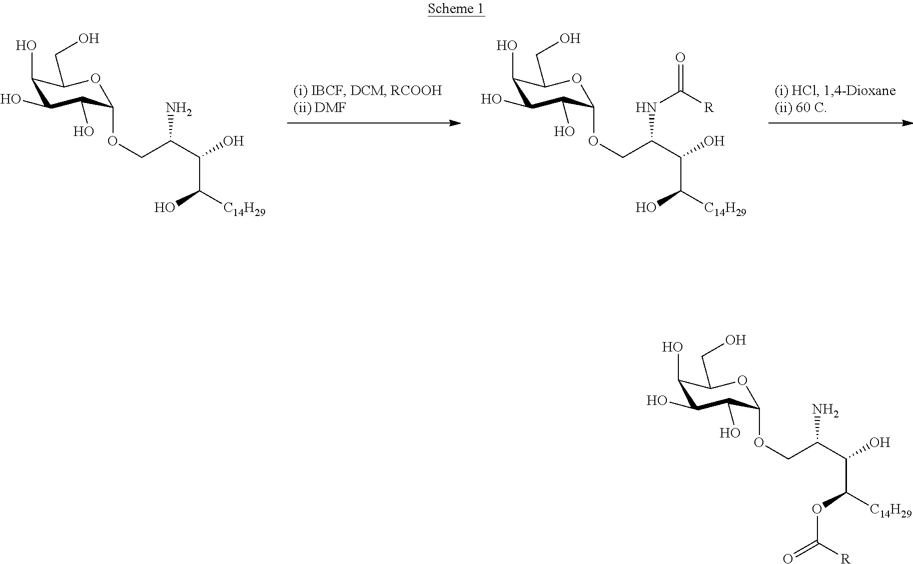

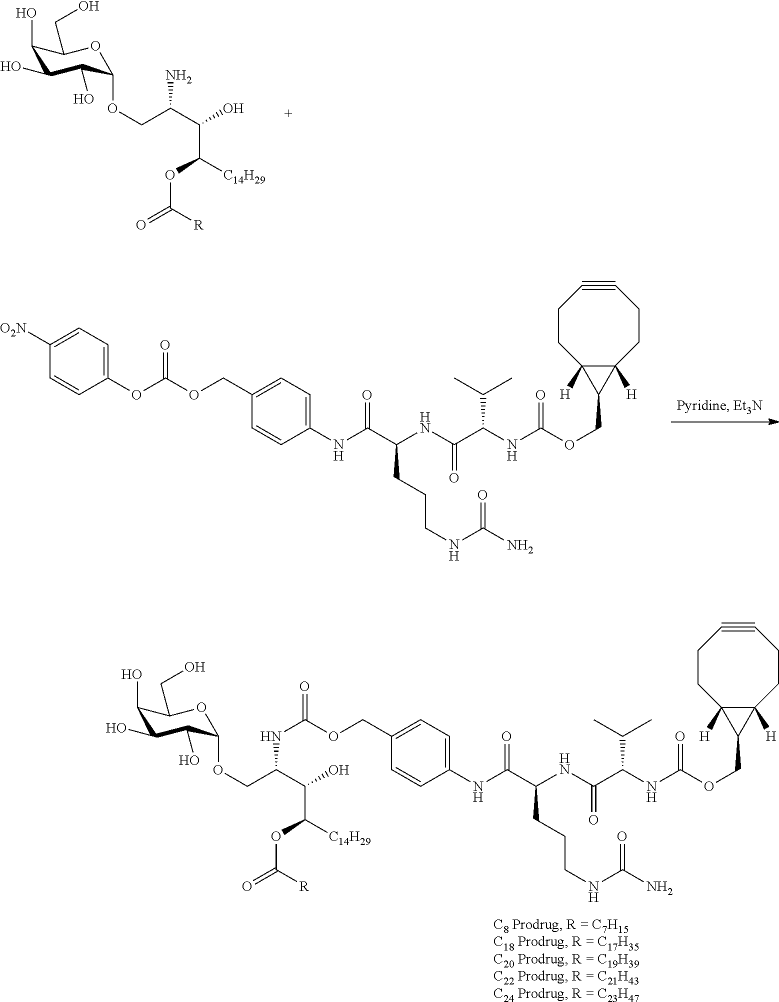

### General Method (B) for Acylation of α-Galactosylphytosphingosine

To a stirred solution of fatty acid (1.2-1.5 equiv) in CH₂Cl₂ was added Et₃N (10 equiv) and IBCF (1.5 equiv) at rt. After 50 min the solution was cooled to 0° C. and added dropwise to a DMF solution of amine (MaGC, 24) (0.1 moles/L) cooled to 0° C. on ice. The reaction was stirred under Ar at rt until complete by TLC and HPLC (A: Water+0.05% TFA, B: MeOH+0.05% TFA; A:B—70:30 to 0:100 over 15 min). The reaction mixture was concentrated under vacuum.

General method (C) for N->O Migration of fatty acyl chain 1,4-Dioxane was freshly distilled from acidified 2,4-dinitrophenylhydrazine. The glycolipid starting material was suspended in 1,4-dioxane (0.1 moles/L) under Ar and heated to 63° C. To this solution was added 37% aqueous HCl (11 equiv) and stirred at 61° C. The reaction was monitored using TLC and HPLC (A: Water+0.05% TFA, B: MeOH+0.05% TFA; A:B—60:40 to 0:100 over 11 min). After 30 min the reaction mixture was concentrated.

### General Method (D) for Linker Attachment

To a stirred mixture of crude amine, obtained from General method C, and pNP-carbonate 11 (1.4 equiv) in anhydrous pyridine (0.1 moles/L with respect to amine) under Ar was added Et₃N (1.4 equiv). The reaction was stirred overnight at rt and monitored using TLC and HPLC (A: Water+0.05% TFA, B: MeOH+0.05% TFA; A:B—70:30 to 0:100 over 12 min). The reaction was then concentrated under vacuum, the solid re-dissolved in 5% MeOH/CHCl₃ and purified using flash chromatography on silica gel.

### General Method (E) for SPAAC Reactions

5-Azidopentanoyl-FFRK-KISQAVHAAHAEINEAGRESIINFEKLTEWT (SEQ ID NO: 1-SEQ ID NO: 11) (68) (1.1 equiv) and cyclooctyne starting material, obtained from General method D, were dissolved in DMSO and the reaction left at rt overnight. Upon completion of the reaction, as monitored by HPLC, the conjugate was purified using C18 preparative HPLC (A: Water+0.05% TFA, B: MeOH+0.05% TFA). The fractions containing the purified conjugate were combined, concentrated and lyophilised.

### (2R,3S,4S,5R,6S)-2-(Acetoxymethyl)-6-(((2S,3S,4R)-3,4-diacetoxy-2-hexacosanamidooctadecyl)oxy)tetrahydro-2H-pyran-3,4,5-triyltriacetate (25)

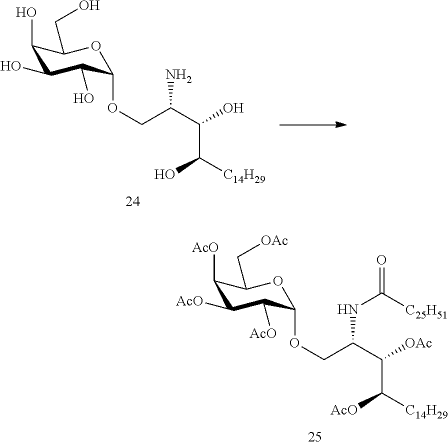

To a stirred solution of cerotic acid (35 mg, 0.087 mmol) in CH₂Cl₂ (0.42 mL) was added Et₃N (84 μL, 0.60 mmol) and IBCF (12 μL, 0.088 mmol). The reaction stirred at rt for 1 h. The solution was then cooled and added dropwise to a cold DMF (0.1 mL) solution of amine 24 (30 mg, 0.058 mmol). The reaction was stirred under Ar at rt for 2.5 h. To the reaction mixture was added acetic anhydride (0.35 mL, 3.6 mmol) and catalytic amount of DMAP (0.8 mg, 0.007 mmol and left stirring overnight. The reaction was diluted with MeOH (5 mL), stirred at rt for 1 h and concentrated under vacuum. The crude product was re-dissolved in 10% EtOAc/petroleum ether and purified using flash chromatography on silica gel (25% EtOAc/petroleum ether) to give compound 25 as a white solid (25 mg, 38%, R_{f}=0.2, 30% EA/PE) ¹H NMR—(500 MHz, 2:1 CDCl₃:CD₃OD) δ 6.53 (d, J=9.7 Hz, 1H), 5.45 (d, J=3.3 Hz, 1H), 5.35 (dd, J=10.8, 3.4 Hz, 1H), 5.31 (dd, J=10.2, 2.4 Hz, 1H), 5.13 (dd, J=10.8, 3.6 Hz, 1H), 4.92 (d, J=3.7 Hz, 1H), 4.90-4.84 (m, 1H), 4.37 (tt, J=10.0, 2.7 Hz, 1H), 4.16-4.08 (m, 2H), 4.06-3.99 (m, 1H), 3.66 (dd, J=10.7, 2.8 Hz, 1H), 3.39 (dd, J=10.7, 2.4 Hz, 1H), 2.30-2.24 (m, 2H), 2.13 (s, 3H), 2.10 (s, 3H), 2.06 (s, 3H), 2.04 (s, 3H), 1.99 (d, J=4.5 Hz, 6H), 1.89-1.79 (m, 2H), 1.70-1.58 (m, 4H), 1.38-1.18 (m, 68H), 0.88 (t, J=6.9 Hz, 6H).

### N-((2S,3S,4R)-3,4-Dihydroxy-1-(((2S,3R,4S,5R,6R)-3,4,5-trihydroxy-6-(hydroxymethyl)tetrahydro-2H-pyran-2-yl)oxy)octadecan-2-yl)hexacosanamide (12)

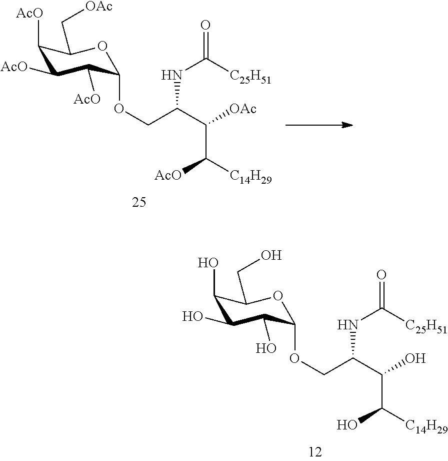

Sodium methoxide (5 μL, 0.027 mmol) was added to a stirred solution of per-OAc-α-GalCer 25 (25 mg, 0.022 mmol) in 1:2 CH₂Cl₂: MeOH (0.6 mL). The reaction was stirred at rt. A white precipitate formed after 2 min into the start of the reaction. After 1.5 h, the reaction was diluted with 1:1 CH₂Cl₂: MeOH (2 mL) and concentrated under vacuum. The crude product was re-dissolved in 10% MeOH/CHCl₃ and purified using flash chromatography on silica gel (10% MeOH/CHCl₃) to give compound 12 as a white solid. (15 mg, 78%, Rₜ=10.2 mins, 99.8% pure by CAD). ¹H NMR—(500 MHz, 2:1 CDCl₃:CD₃OD) δ 4.91 (d, J=3.8 Hz, 1H), 4.23-4.15 (m, 1H), 3.96-3.86 (m, 2H), 3.84-3.65 (m, 6H), 3.59-3.52 (m, 2H), 2.21 (t, J=7.7 Hz, 2H), 1.72-1.51 (m, 4H), 1.38-1.21 (m, 68H), 0.89 (t, J=6.9 Hz, 6H). ¹³C NMR—(126 MHz, 2:1 CDCl₃:CD₃OD) δ 175.0, 100.2, 75.1, 72.4, 71.3, 70.7, 70.2, 69.4, 67.8, 62.2, 50.9, 36.8, 32.9, 32.3, 30.1, 30.07, 30.03, 29.9, 29.8, 29.7, 26.3, 26.2, 23.0, 14.2. HRMS-ESI—calculated for C₅₀H₁₀₀NO₉ [M+H]⁺ 858.7398, observed 858.7398.

### N-((2S,3S,4R)-3,4-Dihydroxy-1-(((2S,3R,4S,5R,6R)-3,4,5-trihydroxy-6-(hydroxymethyl)tetrahydro-2H-pyran-2-yl)oxy)octadecan-2-yl)tetracosanamide (67)

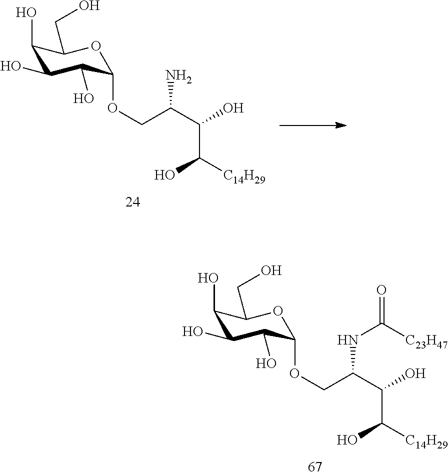

Acylation of the amine 24 (32 mg, 0.066 mmol) with lignoceric acid (38 mg, 0.10 mmol) was carried out using general experimental method B. The crude product was re-dissolved in hot EtOH and cooled at −18° C. to form a precipitate. The well dried precipitate was then re-dissolved in warm 5% MeOH/CHCl₃ and purified using flash chromatography on silica gel (80% MeOH/CHCl₃) to give compound 67 as a white solid (32 mg, 58%, Rₜ=9.7 mins, 99.8% pure by CAD). ¹H NMR—(500 MHz, 2:1 CDCl₃:CD₃OD) δ 4.91 (d, J=3.7 Hz, 1H), 4.24-4.15 (m, 1H), 3.96-3.86 (m, 2H), 3.84-3.66 (m, 6H), 3.59-3.53 (m, 2H), 2.21 (t, J=7.7 Hz, 2H), 1.72-1.50 (m, 4H), 1.42-1.18 (m, 64H), 0.89 (t, J=6.9 Hz, 6H). ¹³C NMR—(126 MHz, 2:1 CDCl₃:CD₃OD) δ 175.0, 100.2, 75.1, 72.4, 71.2, 70.7, 70.2, 69.4, 67.8, 62.2, 50.98, 50.9, 36.9, 36.8, 32.8, 32.3, 30.15, 30.07, 30.04, 29.9, 29.8, 29.7, 26.3, 26.2, 23.0, 14.2. HRMS-ESI—calculated for C₄₈H₉₆NO₉ [M+H]⁺ 830.7085, observed 830.7058.

### N-((2S,3S,4R)-3,4-Dihydroxy-1-(((2S,3R,4S,5R,6R)-3,4,5-trihydroxy-6-(hydroxymethyl)tetrahydro-2H-pyran-2-yl)oxy)octadecan-2-yl)docosanamide (65)

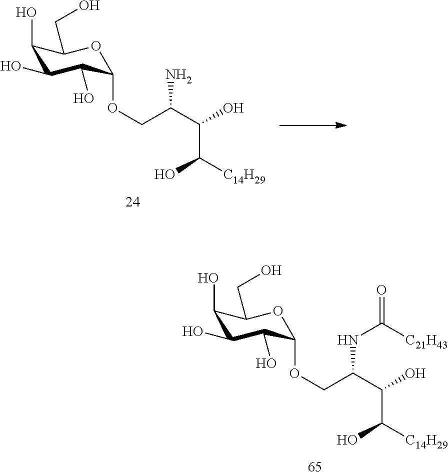

Acylation of the amine 24 (32 mg, 0.066 mmol) with behenic acid (35 mg, 0.10 mmol) was carried out using general experimental method B. The crude product was re-dissolved in warm 50% MeOH/CHCl₃ and dry loaded on silica. The product was purified using flash chromatography on silica gel (21% MeOH/CHCl₃) to give compound 65 as a white solid (26 mg, 49%, Rₜ=9.3 mins, 99.8% pure by CAD). ¹H NMR—(500 MHz, 2:1 CDCl₃:CD₃OD) δ 4.91 (d, J=3.8 Hz, 1H), 4.23-4.17 (m, 1H), 3.96-3.86 (m, 2H), 3.83-3.66 (m, 6H), 3.55 (dd, J=5.7, 2.5 Hz, 2H), 2.21 (t, J=7.7 Hz, 2H), 1.72-1.50 (m, 4H), 1.43-1.19 (m, 60H), 0.89 (t, J=6.9 Hz, 6H). ¹³C NMR—(126 MHz, 2:1 CDCl₃:CD₃OD) δ 175.0, 100.2, 75.1, 72.4, 71.3, 70.7, 70.2, 69.4, 67.8, 62.2, 50.9, 36.8, 32.9, 32.3, 30.17, 30.13, 30.08, 30.07, 30.05, 30.03, 30.01, 29.9, 29.8, 29.76, 29.73, 29.71, 26.28, 26.25, 23.0, 14.2. HRMS-ESI—calculated for C₄₆H₉₂NO₉ [M+H]⁺ 802.6772, observed 802.6784.

### N-((2S,3S,4R)-3,4-Dihydroxy-1-(((2S,3R,4S,5R,6R)-3,4,5-trihydroxy-6-(hydroxymethyl)tetrahydro-2H-pyran-2-yl)oxy)octadecan-2-yl)icosanamide (64)

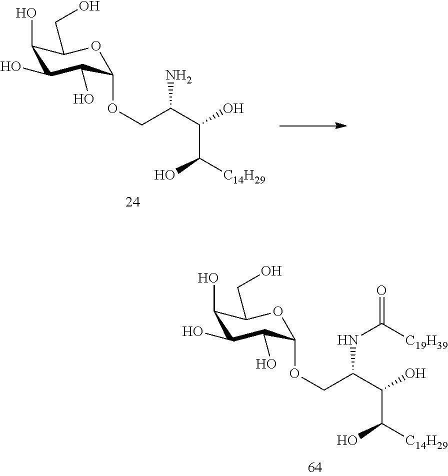

Acylation of the amine 24 (34 mg, 0.071 mmol) with arachidic acid (34 mg, 0.11 mmol) was carried out using general experimental method B with some variations. Upon completion of the reaction, piperidine (0.2 mL, 0.44 mmol) was added to quench the reaction and stirred for another 1 h. The reaction mixture was concentrated under vacuum and the crude product was re-dissolved in warm 50% MeOH/CHCl₃ and dry loaded to silica. The product was purified using flash chromatography on silica gel (22% MeOH/CHCl₃) to give compound 64 as a white solid (23 mg, 42%, Rₜ=9.1 mins, 99.8% pure by CAD). ¹H NMR—(500 MHz, 2:1 CDCl₃:CD₃OD) δ 4.91 (d, J=3.8 Hz, 1H), 4.23-4.17 (m, 1H), 3.96-3.86 (m, 2H), 3.83-3.66 (m, 6H), 3.55 (dd, J=5.7, 2.5 Hz, 2H), 2.21 (t, J=7.7 Hz, 2H), 1.72-1.50 (m, 4H), 1.43-1.19 (m, 56H), 0.89 (t, J=6.9 Hz, 6H). ¹³C NMR—(126 MHz, 2:1 CDCl₃:CD₃OD) δ 175.0, 100.1, 75.0, 72.4, 71.2, 70.6, 70.1, 69.3, 67.7, 62.2, 50.8, 36.8, 32.8, 32.2, 30.1, 30.05, 30.0, 29.9, 29.96, 29.94, 29.9, 29.7, 29.69, 29.65, 29.64, 26.2, 26.17, 23.0, 14.2. HRMS-ESI—calculated for C₄₄H₈₈NO₉ [M+H]⁺ 774.6459, observed 774.6476.

### N-((2S,3S,4R)-3,4-Dihydroxy-1-(((2S,3R,4S,5R,6R)-3,4,5-trihydroxy-6-(hydroxymethyl)tetrahydro-2H-pyran-2-yl)oxy)octadecan-2-yl)stearamide (40)

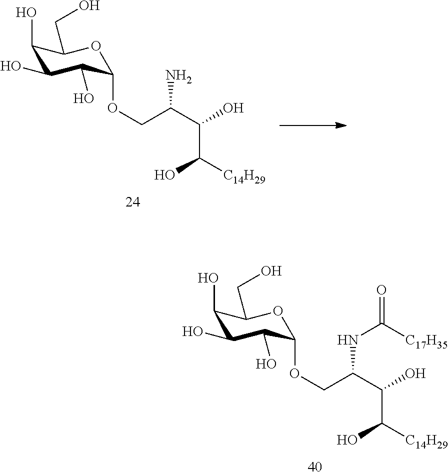

Acylation of the amine 24 (30 mg, 0.058 mmol) with stearic acid (25 mg, 0.088 mmol) was carried out using general experimental method B with some variations. Upon completion of the reaction, piperidine (0.2 mL, 0.44 mmol) was added to quench the reaction and stirred for another 1 h. The reaction mixture was concentrated under vacuum. The crude product was triturated with water, filtered and dried. The crude product was re-dissolved in warm 8% MeOH/CHCl₃ and purified using flash chromatography on silica gel (15% MeOH/CHCl₃) to give compound 40 as a white solid (19 mg, 44%, Rₜ=8.8 mins, 99.8% pure by CAD). ¹H NMR—(500 MHz, 2:1 CDCl₃:CD₃OD) δ 4.90 (d, J=3.8 Hz, 1H), 4.22-4.16 (m, 1H), 3.94 (d, J=3.4 Hz, 1H), 3.89 (dd, J=10.8, 4.5 Hz, 1H), 3.84-3.65 (m, 6H), 3.55 (dd, J=7.8, 2.9 Hz, 2H), 2.21 (t, J=7.7 Hz, 2H), 1.71-1.49 (m, 4H), 1.36-1.22 (m, 52H), 0.89 (t, J=6.9 Hz, 6H). Consistent with literature (Du, 2007). ¹³C NMR—(126 MHz, 2:1 CDCl₃:CD₃OD) δ 175.1, 100.2, 75.1, 72.4, 71.3, 70.7, 70.2, 69.4, 67.8, 62.2, 51.0, 36.8, 32.8, 32.3, 30.2, 30.1, 30.1, 30.0, 29.9, 29.8, 29.77, 29.73, 26.3, 26.25, 23.0, 14.2. HRMS-ESI—calculated for C₄₂H₈₄NO₉ [M+H]+ 746.6146, observed 746.6130.

### N-((2S,3S,4R)-3,4-Dihydroxy-1-(((2S,3R,4S,5R,6R)-3,4,5-trihydroxy-6-(hydroxymethyl)tetrahydro-2H-pyran-2-yl)oxy)octadecan-2-yl)octanamide (37)

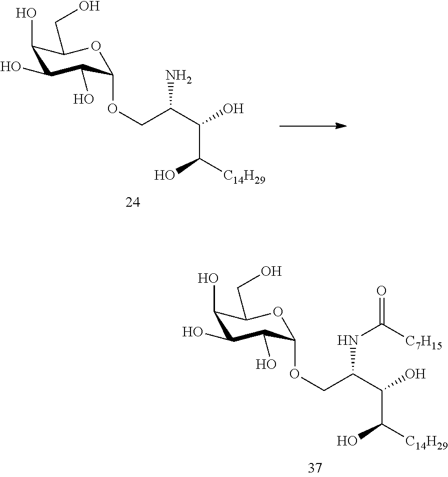

Acylation of the amine 24 (60 mg, 0.12 mmol) with octanoic acid (30 μL, 0.20 mmol) was carried out using general experimental method B with some variations. Upon completion of the reaction, piperidine (0.25 mL, 0.55 mmol) was added to quench the reaction and stirred for another 30 min. The reaction mixture was concentrated under vacuum. The crude product was re-dissolved in 5% MeOH/CHCl₃ and purified using flash chromatography on silica gel (50% MeOH/CHCl₃) to give compound 37 as a white solid (10 mg, 38%, Rₜ=7.7 mins, 99.8% pure by CAD).). ¹H NMR—(500 MHz, 2:1 CDCl₃:CD₃OD) δ 4.91 (d, J=3.8 Hz, 1H), 4.23-4.17 (m, 1H), 3.94 (d, J=3.6 Hz, 1H), 3.89 (dd, J=10.7, 4.7 Hz, 1H), 3.83-3.67 (m, 6H), 3.57-3.53 (m, 2H), 2.21 (t, J=8.4, 6.9 Hz, 2H), 1.72-1.51 (m, 4H), 1.29-1.25 (m, 32H), 0.91-0.86 (m, 6H). ¹³C NMR—(126 MHz, 2:1 CDCl₃:CD₃OD) δ 175.0, 100.1, 75.1, 72.3, 71.2, 70.7, 70.1, 69.3, 67.7, 62.2, 50.8, 36.8, 33.0, 32.2, 32.0, 30.12, 30.1, 30.02, 29.9, 29.7, 29.6, 29.4, 26.2, 26.19, 14.2, 14.1. HRMS-ESI—calculated for C₃₂H₆₄NO₉ [M+H]⁺ 606.4581, observed 606.4574.

### N-((2S,3S,4R)-3,4-Dihydroxy-1-(((2S,3R,4S,5R,6R)-3,4,5-trihydroxy-6-(hydroxymethyl)tetrahydro-2H-pyran-2-yl)oxy)octadecan-2-yl)butyramide (48)

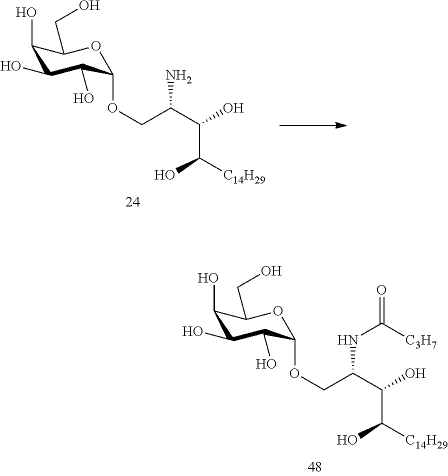

Acylation of the amine 24 (25 mg, 0.048 mmol) with butyric acid (10 μL 0.075 mmol) was carried out using general experimental method B with some variations. Upon completion of the reaction, piperidine (0.2 mL, 0.44 mmol) was added to quench the reaction and stirred for another 1 h. The reaction mixture was concentrated under vacuum and the crude product was re-dissolved in warm 50% MeOH/CHCl₃ and dry loaded to silica. The product was purified using flash chromatography on silica gel (50% MeOH/CHCl₃). Purified product was however, contaminated Et₃N salts which was removed by flash chromatography on C18 silica (100% MeOH/Water) to give compound 48 as a white solid (10 mg, 38%, Rₜ=7.2 mins, 99.8% pure by CAD. rerun LCMS). ¹H NMR—(500 MHz, 2:1 CDCl₃:CD₃OD) δ 4.91 (d, J=3.9 Hz, 1H), 4.24-4.20 (m, 1H), 3.94 (d, J=3.3 Hz, 1H), 3.90 (dd, J=10.8, 4.6 Hz, 1H), 3.83-3.67 (m, 6H), 3.56-3.53 (m, 2H), 2.22-2.17 (m, 2H), 1.66 (dh, J=14.9, 7.4 Hz, 3H), 1.59-1.51 (m, 1H), 1.30-1.25 (m, 24H), 0.96 (t, J=7.4 Hz, 3H), 0.89 (t, J=6.9 Hz, 3H). ¹³C NMR—(126 MHz, 2:1 CDCl₃:CD₃OD) δ 175.0, 100.1, 75.2, 72.4, 71.2, 70.7, 70.1, 69.3, 67.7, 62.2, 50.8, 38.6, 33.0, 32.2, 30.1, 30.04, 30.01, 29.9, 29.7, 26.1, 23.0, 19.5, 14.2, 13.8. HRMS-ESI—calculated for C₂₈H₅₆ NO₉ [M+H]⁺ 550.3955, observed 550.3951.

### (2S,3S,4R)-2-Amino-1-(α-D-galactopyranosyloxy)-3-hydroxy-octadecan-4-yl hexacosanoate (13)

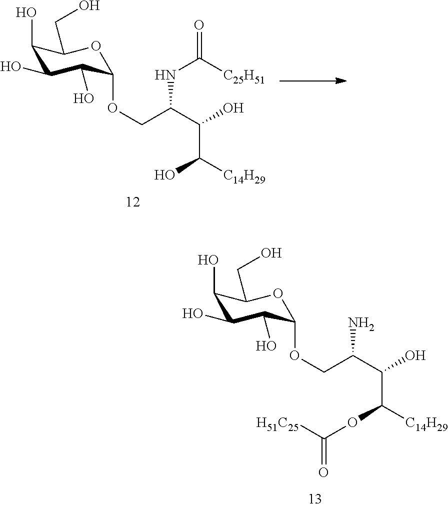

N->O migration of fatty acyl chain of glycolipid 12 (20 mg, 0.023 mmol) was carried out following the general experimental method C. The crude product was re-dissolved in 8% MeOH/CHCl₃ and purified using flash chromatography on silica gel (20% MeOH/CHCl₃) to give migrated product 13 as a white solid (16 mg, 80%, Rₜ=5.2 rains, 99.8% pure by CAD). ¹H NMR—(500 MHz, 2:1 CDCl₃:CD₃OD) δ 4.95-4.86 (m, 2H), 4.14 (d, J=10.5 Hz, 1H), 4.04-3.63 (m, 8H), 3.58 (t, J=9.6 Hz, 1H), 2.43-2.33 (m, 2H), 1.70-1.53 (m, 4H), 1.40-1.19 (m, 68H), 0.89 (t, J=6.9 Hz, 6H). HRMS-ESI—calculated for C₅₀H₁₀₀ NO₉ [M+H]⁺ 858.7389, observed 858.7403.

### (2S,3S,4R)-2-Amino-1-(α-D-galactopyranosyloxy)-3-hydroxy-octadecan-4-yl tetracosanoate (69)

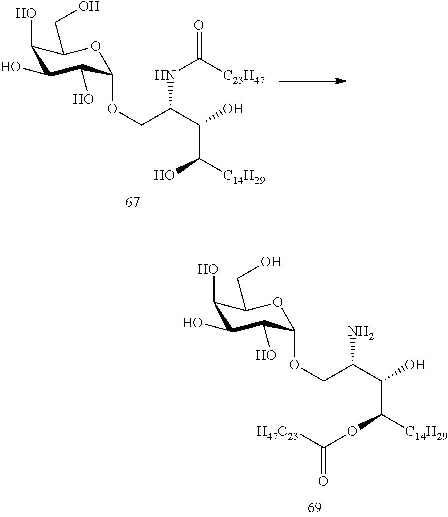

N->O migration of fatty acyl chain of glycolipid 67 (18 mg, 0.022 mmol) was carried out following the general experimental method C. LCMS indicated all starting material converted to migrated product 69 (19 mg, Rₜ=5.1 mins, 96% pure by CAD). ¹H NMR—(500 MHz, 2:1 CDCl₃:CD₃OD) δ 4.97-4.84 (m, 2H), 4.14 (d, J=10.6 Hz, 1H), 4.02-3.63 (m, 8H), 3.57 (t, J=9.7 Hz, 1H), 2.37 (q, J=7.8 Hz, 2H), 1.70-1.53 (m, 4H), 1.37-1.20 (m, 64H), 0.89 (t, J=6.9 Hz, 6H). ¹³C NMR—(126 MHz, 2:1 CDCl₃:CD₃OD) δ 175.0, 100.0, 73.4, 71.2, 70.8, 70.3, 70.2, 69.3, 64.4, 62.2, 53.4, 34.8, 32.3, 31.6, 30.0, 29.9, 29.8, 29.7, 29.5, 25.4, 25.2, 23.0, 14.2. HRMS-ESI—calculated for C₄₈H₉₆ NO₉ [M+H]⁺ 830.7085 observed 830.7071.

### (2S,3S,4R)-2-Amino-1-(α-D-galactopyranosyloxy)-3-hydroxy-octadecan-4-yl docosanoate (70)

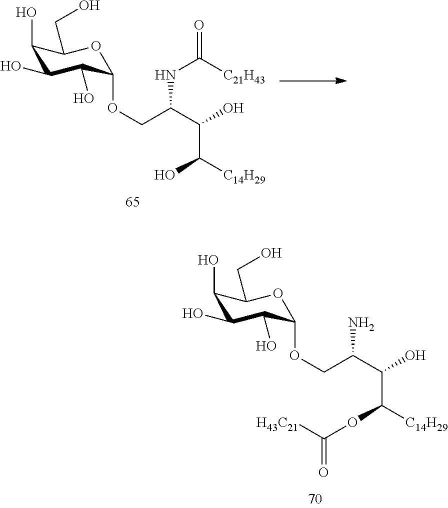

N->O migration of fatty acyl chain of glycolipid 65 (19 mg, 0.024 mmol) was carried out following the general experimental method C. LCMS indicated all starting material converted to migrated product 70 (20 mg, Rₜ=4.95 mins, 95% pure by CAD). ¹H NMR—(500 MHz, 2:1 CDCl₃:CD₃OD) δ 4.9-4.85 (m, 2H), 4.14 (d, J=10.5 Hz, 1H), 4.02-3.63 (m, 8H), 3.57 (t, J=9.8 Hz, 1H), 2.38 (t, J=7.4 Hz, 2H), 1.71-1.54 (m, 4H), 1.40-1.20 (m, 60H), 0.89 (t, J=6.9 Hz, 6H). ¹³C NMR—(126 MHz, 2:1 CDCl₃:CD₃OD) δ 174.5, 100.0, 73.4, 71.3, 70.8, 70.4, 70.2, 69.3, 64.4, 62.2, 53.4, 34.9, 32.2, 31.6, 30.0, 29.9, 29.8, 29.7, 29.6, 25.5, 25.2, 23.0, 14.2. HRMS-ESI—calculated for C₄₆H₉₂NO₉ [M+H]+ 802.6772, observed 802.6774.

### (2S,3S,4R)-2-Amino-1-(α-D-galactopyranosyloxy)-3-hydroxy-octadecan-4-yl icosanoate (71)

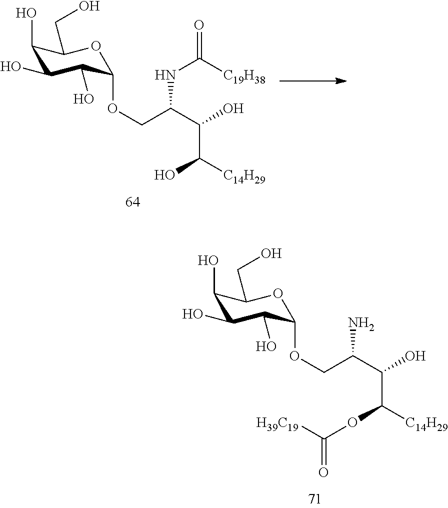

N->O migration of fatty acyl chain of glycolipid 64 (15 mg, 0.019 mmol) was carried out following the general experimental method C. LCMS indicated all starting material converted to migrated product 71 (15 mg, Rₜ=4.9 mins, 95% pure by CAD). ¹H NMR—(500 MHz, 2:1 CDCl₃:CD₃OD) δ 4.98-4.85 (m, 2H), 4.14 (d, J=10.5 Hz, 1H), 4.02-3.63 (m, 8H), 3.61-3.53 (m, 1H), 2.43-2.34 (m, 2H), 1.71-1.53 (m, 4H), 1.39-1.21 (m, 56H), 0.89 (t, J=6.9 Hz, 6H). ¹³C NMR—(126 MHz, 2:1 CDCl₃:CD₃OD) δ 174.4, 100.1, 73.5, 71.3, 71.0, 70.7, 70.4, 70.2, 69.4, 64.4, 62.3, 53.5, 35.0, 32.3, 31.7, 30.1, 30.0, 29.8, 29.76, 29.6, 25.5, 25.3, 23.1, 14.3. HRMS-ESI—calculated for C₄₄H₈₈ NO₉ [M+H]+ 774.6459, observed 774.6465 (2S,3S,4R)-2-Amino-1-(α-D-galactopyranosyloxy)-3-hydroxy-octadecan-4-yl icosanoate (71) stearate (46)

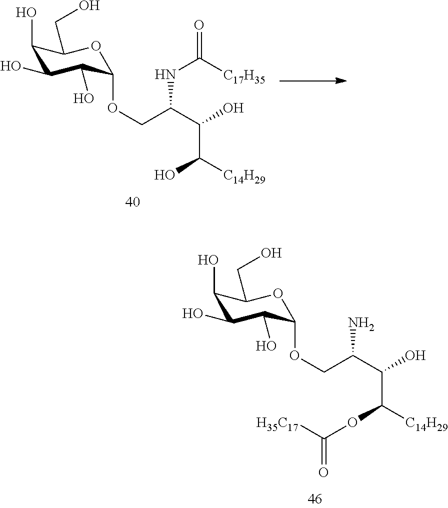

N->O migration of fatty acyl chain of glycolipid 40 (14 mg, 0.018 mmol) was carried out following the general experimental method C. LCMS indicated all starting material converted to migrated product 46 (14 mg, Rₜ=4.7 mins, 89% pure by CAD). ¹H NMR—(500 MHz, 2:1 CDCl₃:CD₃OD) δ 4.96-4.85 (m, 2H), 4.14 (d, J=10.6 Hz, 1H), 4.02-3.62 (m, 8H), 3.57 (t, J=9.8 Hz, 1H), 2.37 (t, J=7.4 Hz, 2H), 1.69-1.53 (m, 4H), 1.36-1.22 (m, 52H), 0.89 (t, J=6.8 Hz, 6H). ¹³C NMR—(126 MHz, 2:1 CDCl₃:CD₃OD) δ 174.5, 100.0, 73.3, 71.1, 70.7, 70.2, 70.1, 69.3, 64.3, 62.2, 53.3, 34.7, 32.2, 31.6, 30.0, 29.7, 29.6, 29.5, 25.3, 25.2, 23.0, 14.2 HRMS-ESI—calculated for C₄₂H₈₄ NO₉ [M+H]⁺ 746.6146, observed 746.6150 (2S,3S,4R)-2-Amino-1-(α-D-galactopyranosyloxy)-3-hydroxy-octadecan-4-yl icosanoate (71) octanoate (44)

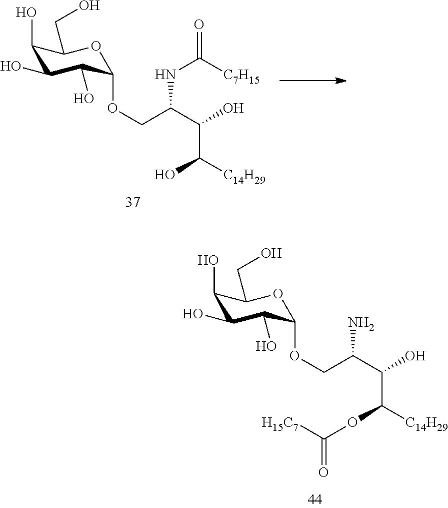

N->O migration of fatty acyl chain of glycolipid 37 (22 mg, 0.036 mmol) was carried out following the general experimental method C. LCMS indicated all starting material converted to migrated product 44 (23 mg, Rₜ=3.8 mins, 87% pure by CAD). ¹H NMR—(500 MHz, 2:1 CDCl₃:CD₃OD) δ 4.96-4.86 (m, 2H), 4.14 (d, J=10.6 Hz, 1H), 4.03-3.68 (m, 8H), 3.58 (t, J=10.2 Hz, 1H), 2.38 (t, J=7.4 Hz, 2H), 1.69-1.52 (m, 4H), 1.37-1.22 (m, 32H), 0.94-0.85 (m, 6H). ¹³C NMR—(126 MHz, 2:1 CDCl₃:CD₃OD) δ 174.5, 100.0, 73.4, 71.2, 70.7, 70.2, 70.1, 69.3, 64.3, 62.2, 53.3, 34.7, 32.2, 32.0, 31.6, 30.0, 29.95, 29.9, 29.7, 29.6, 29.4, 29.26, 29.2, 25.4, 25.2, 23.0, 22.9, 14.2, 14.1. HRMS-ESI—calculated for C₃₂H₆₄NO₉ [M+H]⁺ 606.4581, observed 606.4585.

### (2S,3S,4R)-2-Amino-1-(α-D-galactopyranosyloxy)-3-hydroxy-octadecan-4-yl icosanoate (71) butyrate (49)

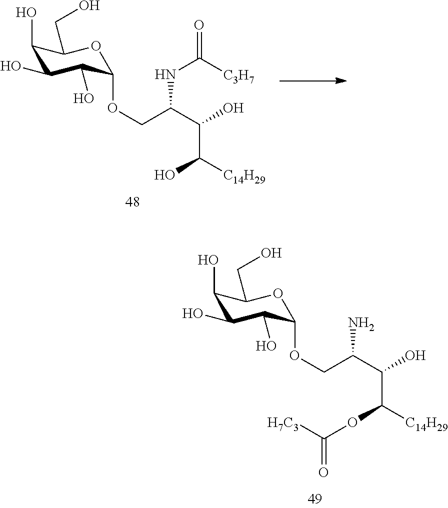

N->O migration of fatty acyl chain of glycolipid 48 (9 mg, 0.016 mmol) was carried out following the general experimental method C. LCMS indicated all starting material converted to migrated product 49 (9 mg, Rₜ=3.4 mins, 96% pure by CAD). ¹H NMR—(500 MHz, 2:1 CDCl₃:CD₃OD) δ 4.96-4.85 (m, 2H), 4.15 (d, J=10.6 Hz, 1H), 4.04-3.64 (m, 8H), 3.57 (t, J=9.8 Hz, 1H), 2.36 (t, J=7.3 Hz, 2H), 1.88-1.75 (m, 1H), 1.71-1.62 (m, 2H), 1.57 (d, J=11.4 Hz, 2H), 1.36-1.23 (m, 24H), 0.98 (t, J=7.2 Hz, 3H), 0.89 (t, J=6.9 Hz, 3H). ¹³C NMR—(126 MHz, 2:1 CDCl₃:CD₃OD) δ 174.3, 100.0, 73.4, 71.2, 70.8, 70.3, 70.1, 69.3, 64.3, 62.2, 53.4, 36.5, 32.2, 31.6, 29.97, 29.94, 29.8, 29.7, 29.6, 25.2, 23.0, 18.7, 14.2, 13.8. HRMS-ESI—calculated for C₂₈H₅₆ NO₉ [M+H]⁺ 550.3955, observed 550.3950.

### (2S,3S,4R)-2-(N-((Bicyclo[6.1.0]non-4-yn-9-yl)methoxycarbonyl)-L-valinyl-L-citrullinyl-4-aminobenzyloxycarbonylamino)-1-(α-D-galactopyranosyloxy)-3-hydroxy-octadecan-4-yl hexacosanoate (MaGC-PAB-cyclooctyne 14)

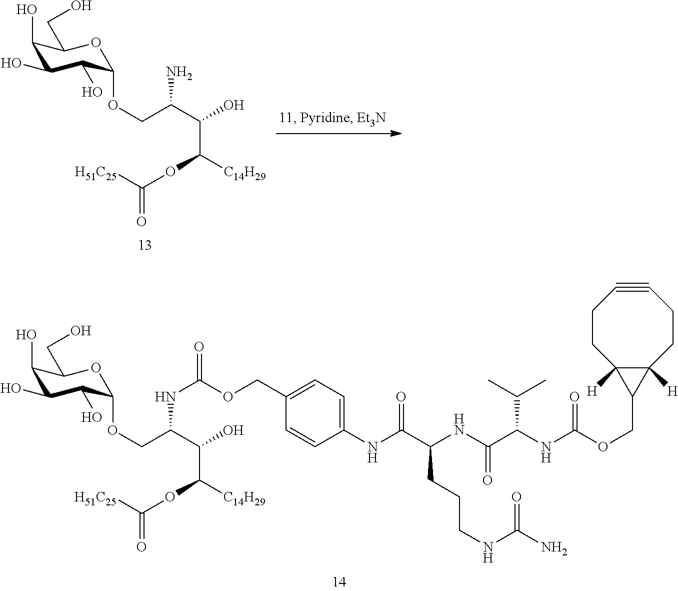

pNP-carbonate linker was attached to the purified amine 13 starting material (16 mg, 0.019 mmol) following the general experimental method D. The crude product was purified (8% MeOH/CHCl₃) to give compound 14 as a white solid (15 mg, 56%, Rₜ=9.1 mins, 99.2% pure by UV 254 nm). ¹H NMR—(500 MHz, 2:3 CDCl₃:CD₃OD) δ 7.57 (d, J=8.3 Hz, 2H), 7.32 (d, J=8.4 Hz, 2H), 5.18-5.09 (m, 1H), 5.03-4.91 (m, 2H), 4.85 (d, J=3.8 Hz, 1H), 4.61-4.51 (m, 1H), 4.08-3.91 (m, 3H), 3.90-3.84 (m, 2H), 3.82-3.64 (m, 8H), 3.29-3.18 (m, 1H), 3.12 (m, 1H), 2.47-2.21 (m, 5H), 2.19-2.03 (m, 3H), 1.97-1.84 (m, 1H), 1.79-1.50 (m, 7H), 1.41-1.20 (m, 70H), 0.99 (d, J=6.8 Hz, 3H), 0.95 (d, J=6.8 Hz, 3H), 0.89 (t, J=6.9 Hz, 6H), 0.81-0.66 (m, 3H). ¹³C NMR—(126 MHz, 2:3 CDCl₃:CD₃OD) δ 175.0, 173.2, 171.0, 161.0, 157.8, 157.0, 138.2, 129.0, 120.4, 100.4, 99.1, 75.0, 72.3, 71.0, 70.6, 70.2, 69.4, 68.4, 66.8, 62.3, 61.0, 53.7, 52.6, 39.3, 35.0, 33.5, 32.2, 31.3, 30.0, 29.98, 29.75, 29.7, 29.5, 29.2, 26.7, 25.6, 25.4, 24.0, 23.4, 23.3, 23.0, 21.6, 19.0, 18.0, 14.2 HRMS-ESI—calculated for C₈₀H₁₃₉N₆O₁₆ [M+H]⁺ 1440.0248, observed 1440.0259.

### (2S,3S,4R)-2-(N-((Bicyclo[6.1.0]non-4-yn-9-yl)methoxycarbonyl)-L-valinyl-L-citrullinyl-4-aminobenzyloxycarbonylamino)-1-(α-D-galactopyranosyloxy)-3-hydroxy-octadecan-4-yl tetracosanoate (72)

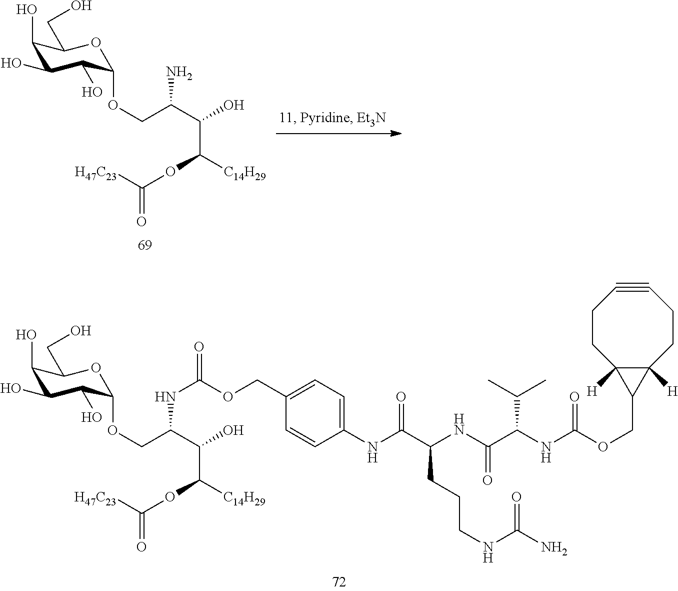

pNP-carbonate linker was attached to the crude amine starting material 69 (18 mg, 0.022 mmol) following the general experimental method D and purified (17% MeOH/CHCl₃). The purified compound 72 had traces of Et₃N salts which were removed by trituration with water to give a white solid (22 mg, 72% over two steps, calculated from starting material 67, Rₜ=8.3 mins, 98% pure by UV 254 nm). ¹H NMR—(500 MHz, 2:3 CDCl₃:CD₃OD) δ 7.57 (d, J=8.1 Hz, 2H), 7.32 (d, J=8.2 Hz, 2H), 6.55 (d, exch, J=8.9 Hz, 0.1H), 5.13 (d, J=12.2 Hz, 1H), 5.03-4.93 (m, 2H), 4.85 (d, J=3.9 Hz, 1H), 4.62-4.51 (m, 1H), 4.07-3.92 (m, 4H), 3.90-3.65 (m, 9H), 3.30-3.20 (m, 1H), 3.19-3.07 (m, 1H), 2.47-2.05 (m, 9H), 1.98-1.87 (m, 1H), 1.80-1.47 (m, 7H), 1.27 (s, 66H), 0.99 (d, J=6.8 Hz, 3H), 0.95 (d, J=6.7 Hz, 3H), 0.89 (t, J=6.9 Hz, 6H), 0.80-0.66 (m, 3H). ¹³C NMR—(126 MHz, 2:3 CDCl₃:CD₃OD) δ 175.0, 173.2, 171.0, 161.0, 158.0, 157.1, 138.2, 133.0, 129.0, 125.6, 120.5, 100.4, 99.1, 97.0, 75.0, 72.3, 71.0, 70.6, 70.2, 70.0, 69.4, 68.4, 66.8, 62.3, 61.0, 53.7, 52.6, 47.0, 46.6, 39.4, 35.0, 33.6, 32.28, 32.26, 31.4, 30.05, 30.01, 30.0, 29.94, 29.91, 29.9, 29.8, 29.7, 29.68, 29.5, 29.2, 26.7, 25.7, 25.4, 24.0, 23.4, 23.3, 23.0, 21.5, 19.4, 18.0, 14.23, 14.21. HRMS-ESI—calculated for C₇₈H₁₃₅N₆O₁₆ [M+H]⁺ 1411.9929, observed 1411.9909.

### (2S,3S,4R)-2-(N-((Bicyclo[6.1.0]non-4-yn-9-yl)methoxycarbonyl)-L-valinyl-L-citrullinyl-4-aminobenzyloxycarbonylamino)-1-(α-D-galactopyranosyloxy)-3-hydroxy-octadecan-4-yl docosanoate (73)

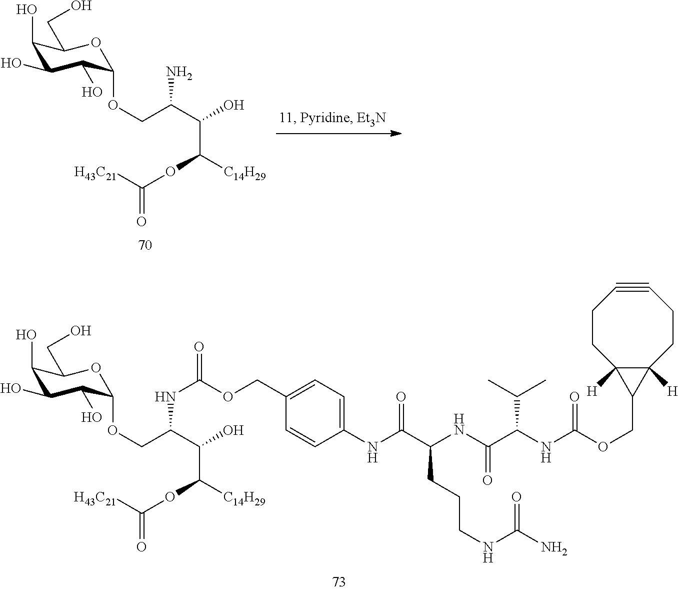

pNP-carbonate linker was attached to the crude amine starting material 70 (19 mg, 0.024 mmol) following the general experimental method D and purified (18% MeOH/CHCl₃). The purified compound 73 had traces of Et₃N salts which were removed by trituration with water to give a white solid (14 mg, 43% over two steps, calculated from starting material 65, Rₜ=7.9 mins, 98% pure by UV 254 nm). ¹H NMR—(500 MHz, 2:3 CDCl₃:CD₃OD) δ 7.57 (d, J=8.2 Hz, 2H), 7.35-7.28 (m, 2H), 5.13 (d, J=12.3 Hz, 1H), 5.03-4.93 (m, 2H), 4.85 (d, J=3.9 Hz, 1H), 4.61-4.49 (m, 1H), 4.06-3.83 (m, 5H), 3.82-3.64 (m, 8H), 3.29-3.20 (m, 1H), 3.19-3.07 (m, 1H), 2.47-2.05 (m, 9H), 1.98-1.86 (m, 1H), 1.80-1.48 (m, 7H), 1.40-1.21 (m, 66H), 0.99 (d, J=6.7 Hz, 3H), 0.94 (d, J=6.8 Hz, 3H), 0.89 (t, J=6.9 Hz, 6H), 0.80-0.65 (m, 3H). ¹³C NMR—(126 MHz, 2:3 CDCl₃:CD₃OD) δ 175.0, 171.0, 161.0, 157.0, 138.2, 133.0, 129.0, 120.4, 100.3, 99.1, 75.0, 72.3, 71.0, 70.6, 70.2, 70.0, 69.4, 68.4, 66.8, 62.3, 61.0, 53.7, 52.5, 47.0, 33.5, 32.23, 32.22, 31.3, 30.01, 30.0, 29.94, 29.9, 29.86, 29.74, 29.7, 29.6, 29.5, 29.2, 26.7, 25.6, 25.4, 24.0, 23.4, 23.3, 23.0, 21.5, 19.4, 18.0, 14.2. HRMS-ESI—calculated for C₇₆H₁₃₂N₆O₁₆ [M+H]⁺ 1383.9616, observed 1383.9614.

### (2S,3S,4R)-2-(N-((Bicyclo[6.1.0]non-4-yn-9-yl)methoxycarbonyl)-L-valinyl-L-citrullinyl-4-aminobenzyloxycarbonylamino)-1-(α-D-galactopyranosyloxy)-3-hydroxy-octadecan-4-yl icosanoate (74)

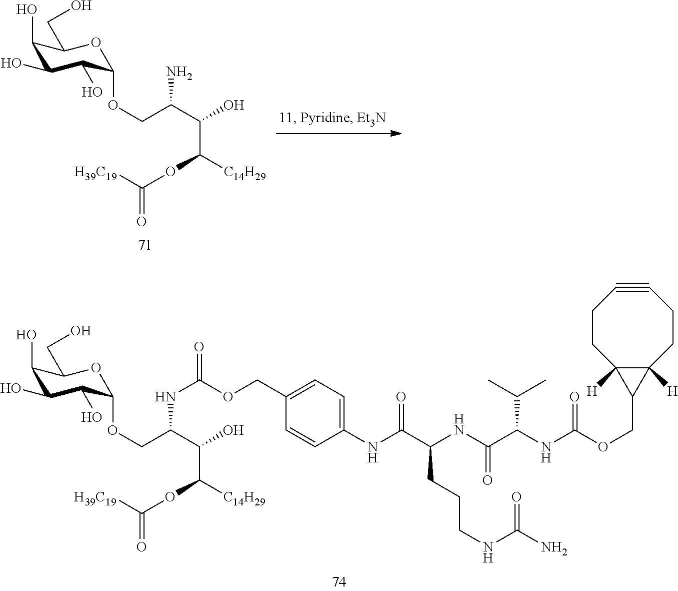

pNP-carbonate linker was attached to the crude amine starting material 71 (15 mg, 0.019 mmol) following the general experimental method D. The crude product was purified (12% MeOH/CHCl₃) to give compound 74 as a white solid (16 mg, 61% over two steps, calculated from starting material 64, Rₜ=7.5 mins, 98% pure by UV 254 nm). ¹H NMR—(500 MHz, 2:3 CDCl₃:CD₃OD) δ 7.57 (d, J=8.2 Hz, 2H), 7.32 (d, J=8.1 Hz, 2H), 5.13 (d, J=12.3 Hz, 1H), 5.03-4.93 (m, 2H), 4.85 (d, J=3.8 Hz, 1H), 4.63-4.50 (m, 1H), 4.08-3.83 (m, 5H), 3.83-3.63 (m, 8H), 3.29-3.19 (m, 1H), 3.16-3.07 (m, 1H), 2.47-2.04 (m, 9H), 1.98-1.85 (m, 1H), 1.81-1.47 (m, 7H), 1.39-1.16 (m, 58H), 0.99 (d, J=6.7 Hz, 3H), 0.95 (d, J=6.8 Hz, 3H), 0.89 (t, J=6.9 Hz, 6H), 0.79-0.66 (m, 3H). ¹³C NMR—(126 MHz, 2:3 CDCl₃:CD₃OD) δ 175.0, 173.2, 171.0, 161.1, 158.0, 157.1, 138.2, 129.1, 120.5, 100.4, 99.1, 75.0, 72.3, 71.0, 70.7, 70.2, 70.0, 69.4, 68.4, 66.8, 62.3, 61.0, 53.7, 52.6, 39.4, 35.0, 33.6, 32.3, 31.4, 30.05, 30.02, 29.93, 29.9, 29.7, 29.68, 29.5, 29.2, 26.8, 25.7, 25.4, 24.0, 23.4, 23.3, 23.0, 21.5, 19.4, 18.0, 14.2. HRMS-ESI—calculated for C₇₄H₁₂₇N₆O₁₆ [M+H]⁺ 1355.9303, observed 1355.9304.

### (2S,3S,4R)-2-(N-((Bicyclo[6.1.0]non-4-yn-9-yl)methoxycarbonyl)-L-valinyl-L-citrullinyl-4-aminobenzyloxycarbonylamino)-1-(α-D-galactopyranosyloxy)-3-hydroxy-octadecan-4-yl stearate (47)

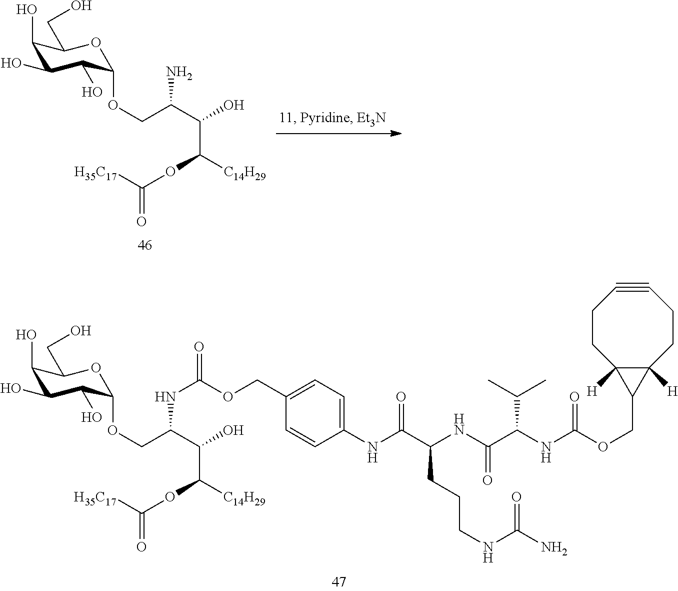

pNP-carbonate linker was attached to the crude amine starting material 46 (14 mg, 0.019 mmol) following the general experimental method D. The crude product was purified (38% MeOH/CHCl₃) to give compound 47 as a white solid (9 mg, 36% over two steps, calculated from starting material 40, Rₜ=7.3 mins, 89% pure by UV 254 nm). ¹H NMR—(500 MHz, 2:3 CDCl₃:CD₃OD) δ 7.57 (d, J=8.2 Hz, 2H), 7.32 (d, J=8.1 Hz, 2H), 5.13 (d, J=12.4 Hz, 1H), 5.03-4.90 (m, 2H), 4.85 (d, J=3.8 Hz, 1H), 4.57 (d, J=18.3 Hz, 1H), 4.07-3.83 (m, 5H), 3.83-3.63 (m, 8H), 3.30-3.19 (m, 1H), 3.18-3.07 (m, 1H), 2.48-2.05 (m, 8H), 1.98-1.87 (m, 1H), 1.80-1.48 (m, 8H), 1.44-1.11 (m, 54H), 0.99 (d, J=6.8 Hz, 3H), 0.95 (d, J=6.8 Hz, 3H), 0.89 (t, J=6.9 Hz, 6H), 0.80-0.66 (m, 3H). ¹³C NMR—(126 MHz, 2:3 CDCl₃:CD₃OD) δ 175.0, 171.0, 158.0, 157.0, 138.2, 133.0, 129.1, 120.4, 100.3, 99.1, 75.0, 72.3, 71.0, 70.6, 70.2, 70.0, 69.4, 68.4, 67.0, 61.0, 52.6, 39.4, 35.0, 33.5, 32.2, 31.3, 30.0, 29.9, 29.75, 29.7, 29.5, 29.2, 26.7, 25.6, 25.4, 24.0, 23.36, 23.3, 23.0, 21.5, 19.4, 18.4, 18.01, 14.2. HRMS-ESI—calculated for C₇₂H₁₂₃N₆O₁₆ [M+H]⁺ 1327.8996, observed 1327.8990.

### (2S,3S,4R)-2-(N-((Bicyclo[6.1.0]non-4-yn-9-yl)methoxycarbonyl)-L-valinyl-L-citrullinyl-4-aminobenzyloxycarbonylamino)-1-(α-D-galactopyranosyloxy)-3-hydroxy-octadecan-4-yl octanoate

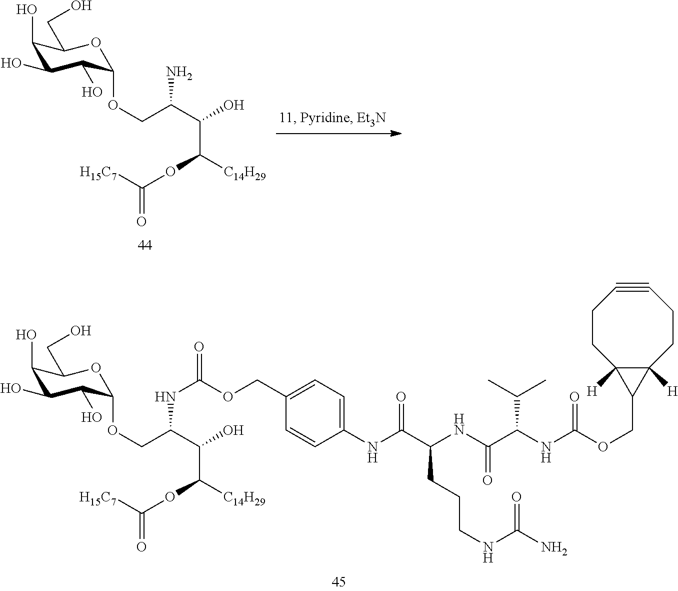

pNP-carbonate linker was attached to the crude amine starting material 44 (22 mg, 0.036 mmol) following the general experimental method D. The crude product was purified (20% MeOH/CHCl₃) to give compound 45 as a white solid (13 mg, 30% over two steps, calculated from starting material 37, Rₜ=6.4 mins, 99.8% pure by UV 254 nm). ¹H NMR—(500 MHz, 2:3 CDCl₃:CD₃OD) δ 7.57 (d, J=8.3 Hz, 2H), 7.32 (d, J=8.3 Hz, 2H), 5.12 (d, J=12.3 Hz, 1H), 5.03-4.91 (m, 2H), 4.85 (d, J=4.0 Hz, 1H), 4.62-4.50 (m, 1H), 4.07-3.83 (m, 5H), 3.83-3.64 (m, 8H), 3.28-3.20 (m, 1H), 3.16-3.07 (m, 1H), 2.46-2.05 (m, 9H), 1.98-1.86 (m, 1H), 1.80-1.48 (m, 7H), 1.41-1.20 (m, 34H), 0.98 (d, J=6.8 Hz, 3H), 0.94 (d, J=6.8 Hz, 3H), 0.92-0.86 (m, 6H), 0.79-0.67 (m, 3H). ¹³C NMR—(126 MHz, 2:3 CDCl₃:CD₃OD) δ 175.0, 173.1, 171.0, 161.0, 158.0, 157.0, 138.2, 133.0, 129.1, 120.4, 100.3, 99.1, 75.0, 72.3, 71.0, 70.6, 70.2, 70.0, 69.4, 68.4, 66.7, 62.3, 61.0, 53.7, 52.5, 39.4, 35.0, 33.5, 32.2, 32.0, 31.3, 30.0, 29.91, 29.9, 29.75, 29.7, 29.4, 29.3, 29.2, 26.7, 25.6, 25.4, 24.0, 23.3, 23.0, 21.5, 19.4, 18.01, 14.2, 14.2. HRMS-ESI—calculated for C₆₂H₁₀₃N₆O₁₆ [M+H]⁺ 1187.7431, observed 1187.7432.

### (2S,3S,4R)-2-(N-((Bicyclo[6.1.0]non-4-yn-9-yl)methoxycarbonyl)-L-valinyl-L-citrullinyl-4-aminobenzyloxycarbonylamino)-1-(α-D-galactopyranosyloxy)-3-hydroxy-octadecan-4-yl butyrate (50)

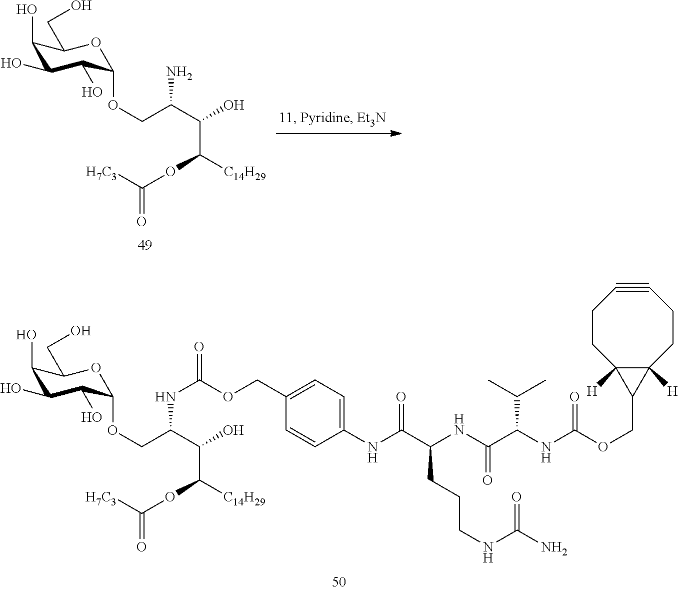

pNP-carbonate linker was attached to the crude amine starting material 49 (9 mg, 0.016 mmol) following the general experimental method D. The crude product was purified (20% MeOH/CHCl₃). Traces of Et₃N salts were present which was removed by column chromatography on C18 silica (100% MeOH/Water) to give purified product 50 (8 mg, 43% over two steps, calculated from starting material 48, Rₜ=6.0 mins, 99.8% pure by UV 254 nm). ¹H NMR—(500 MHz, 2:3 CDCl₃:CD₃OD) δ 7.57 (d, J=8.3 Hz, 2H), 7.32 (d, J=8.5 Hz, 2H), 5.11 (d, J=12.3 Hz, 1H), 5.04-4.93 (m, 2H), 4.85 (d, J=3.8 Hz, 1H), 4.60-4.52 (m, 1H), 4.06-3.83 (m, 5H), 3.82-3.65 (m, 8H), 3.29-3.20 (m, 1H), 3.17-3.06 (m, 1H), 2.45-2.06 (m, 9H), 2.00-1.86 (m, 1H), 1.79-1.48 (m, 7H), 1.35-1.22 (m, 26H), 1.01-0.92 (m, 9H), 0.92-0.86 (m, 3H), 0.80-0.66 (m, 3H). ¹³C NMR—(126 MHz, 2:3 CDCl₃:CD₃OD) δ 174.5, 173.3, 171.2, 161.2, 157.4, 138.4, 129.0, 120.5, 100.4, 99.2, 75.2, 72.3, 71.0, 70.7, 70.2, 70.0, 69.4, 68.4, 66.8, 62.2, 61.0, 53.8, 52.7, 40.4, 36.8, 33.6, 32.3, 31.4, 30.05, 30.01, 29.94, 29.9, 29.8, 29.7, 26.8, 25.7, 24.0, 23.43, 23.4, 23.0, 21.6, 19.4, 19.0, 14.2, 13.8. HRMS-ESI—calculated for C₅₈H₉₅N₆O₁₆ [M+H]⁺ 1131.6805, observed 1131.6801.

V.S.FFRK.OVA_{LP} C₂₆ Conjugate (54)

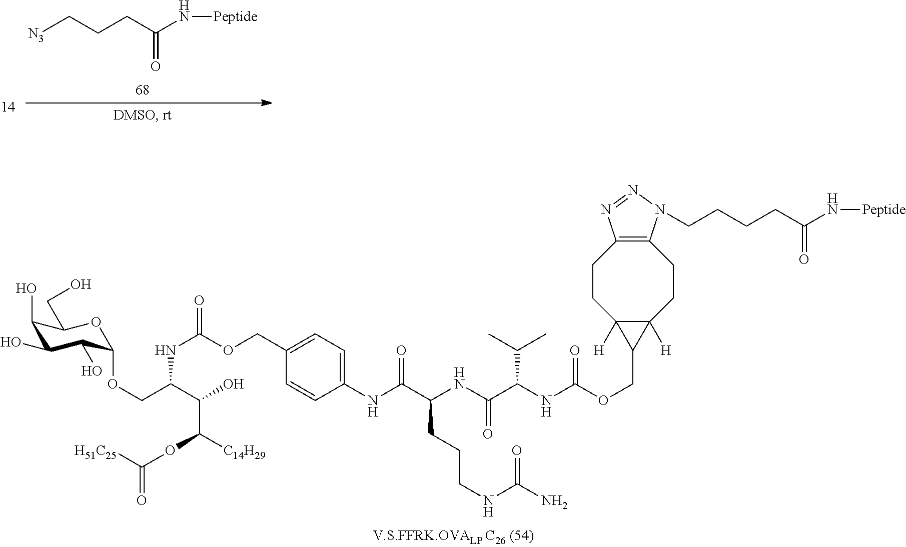

The cyclooctyne starting material 14 (2.0 mg, 0.0014 mmol) was coupled to 5-azidopentanoyl-FFRK-KISQAVHAAHAEINEAGRESIINFEKLTEWT (SEQ ID NO: 1-SEQ ID NO: 11) following the general experimental method E. The conjugate 54 was purified (A:B—70:30 to 0:100 over 14 min) and obtained as a white coloured fluffy solid (5.4 mg, 69%, 99.9% pure by UV 254 nm, Rₜ=6.2 mins). HRMS-ESI—calculated for C₂₆₉H₄₃₃N₆₁O₇₀ [M+4H]⁴⁺ 1410.3071, observed 1410.3015.

V.S.FFRK.OVA_{LP} C₂₄ Conjugate (75)

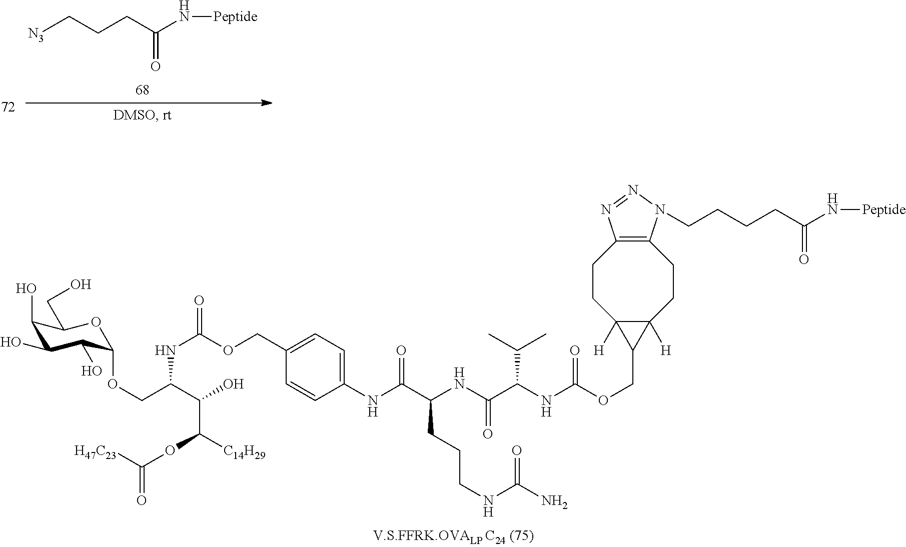

The cyclooctyne starting material 72 (1.2 mg, 0.00085 mmol) was coupled to the peptide following the general experimental method E. The conjugate 75 was purified (A:B—70:30 to 0:100 over 14 min) and obtained as a white coloured fluffy solid (2.7 mg, 57%, 99.9% pure by UV 254 nm, Rₜ=6.1 mins). HRMS-ESI—calculated for C₂₆₇H₄₃₀N₆₁O₇₀[M+5H]⁵⁺ 1122.8411, observed 1122.8387.

V.S.FFRK.OVA_{LP} C₂₂ Conjugate (76)

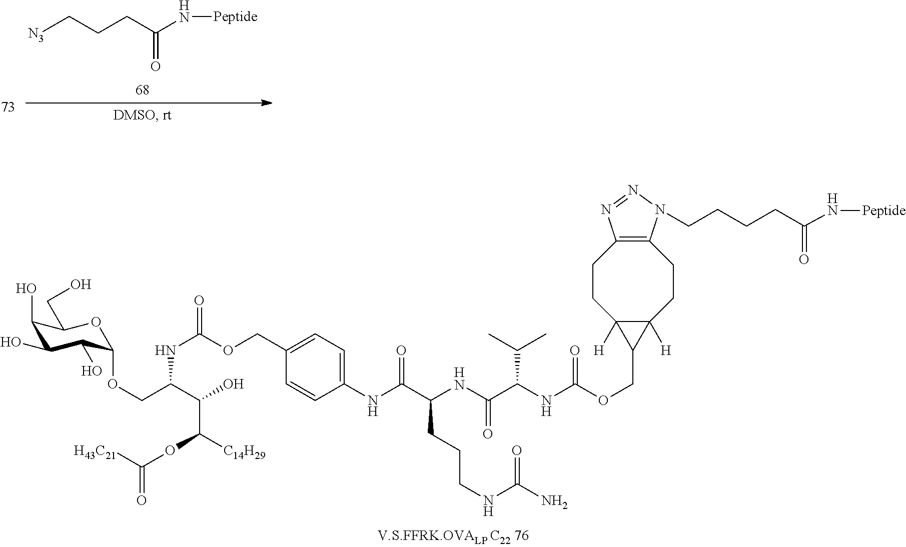

The cyclooctyne starting material 73 (1.3 mg, 0.00094 mmol) was coupled to the peptide following the general experimental method E. The conjugate 76 was purified (A:B—70:30 to 0:100 over 14 min) and obtained as a white coloured fluffy solid (4.1 mg, 78%, 99.9% pure by UV 254 nm, Rₜ=6.0 rains). HRMS-ESI—calculated for C₂₆₅H₄₂₆N₆₁O₇₀ [M+5H]⁵⁺ 1117.2347, observed 1117.2301.

V.S.FFRK.OVA_{LP} C₂₀ Conjugate (77)

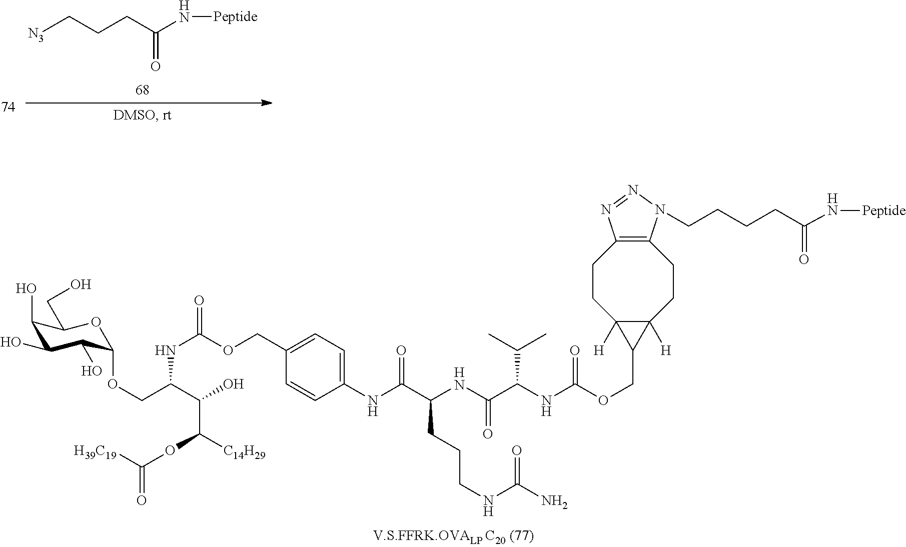

The cyclooctyne starting material 74 (1.2 mg, 0.00089 mmol) was coupled to the peptide following the general experimental method E. The conjugate 77 was purified (A:B—70:30 to 0:100 over 14 min) and obtained as a white coloured fluffy solid (0.85 mg, 17%, 99.9% pure by UV 254 nm, Rₜ=5.9 rains). HRMS-ESI—calculated for C₂₆₃H₄₂₁N₆₁O₇₀ [M+4H]⁴⁺ 1389.2837, observed 1389.2781.

### V.S.FFRK.OVA_{LP} Cis Conjugate (51)

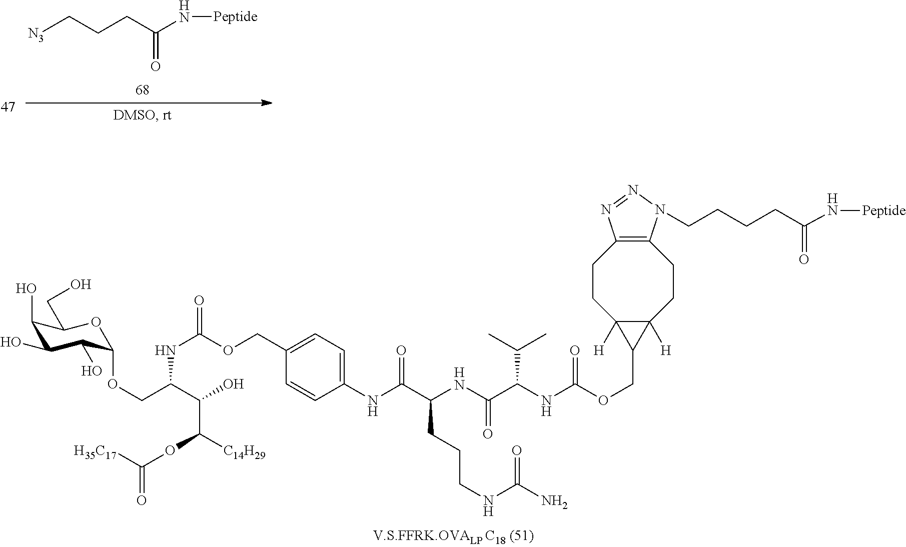

The cyclooctyne starting material 47 (2.0 mg, 0.0015 mmol) was coupled to the peptide following the general experimental method E. The conjugate 51 was purified (A:B—70:30 to 0:100 over 14 min) and obtained as a white coloured fluffy solid (4.12 mg, 49%, 99.9% pure by UV 254 nm, Rₜ=5.9 rains). HRMS-ESI—calculated for C₂₆₁H₄₁₈N₆₁O₇₀ [M+5H]⁵⁺ 1106.0222, observed 1106.0193.

V.S.FFRK.OVA_{LP} C₈ Conjugate (52)

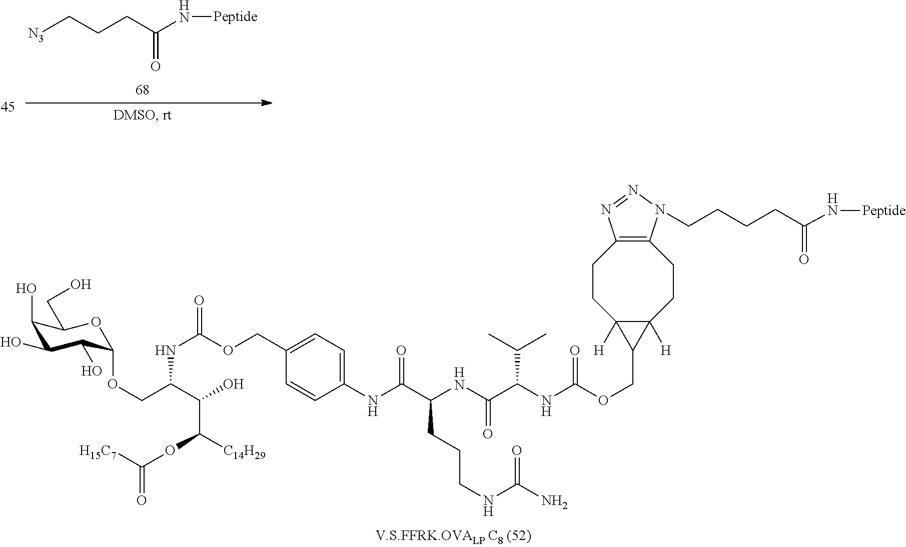

The cyclooctyne starting material 45 (2.0 mg, 0.0017 mmol) was coupled to the peptide following the general experimental method E. The conjugate 52 was purified (A:B—70:30 to 0:100 over 13 min) and obtained as a white coloured fluffy solid (3.3 mg, 36%, 99.9% pure by UV 254 nm, Rₜ=5.45 rains). HRMS-ESI—calculated for C₂₅₁H₃₉₈N₆₁O₇₀ [M+5H]⁵⁺ 1077.9910, observed 1077.9894.

V.S.FFRK.OVA_{LP} C₄ Conjugate (53)

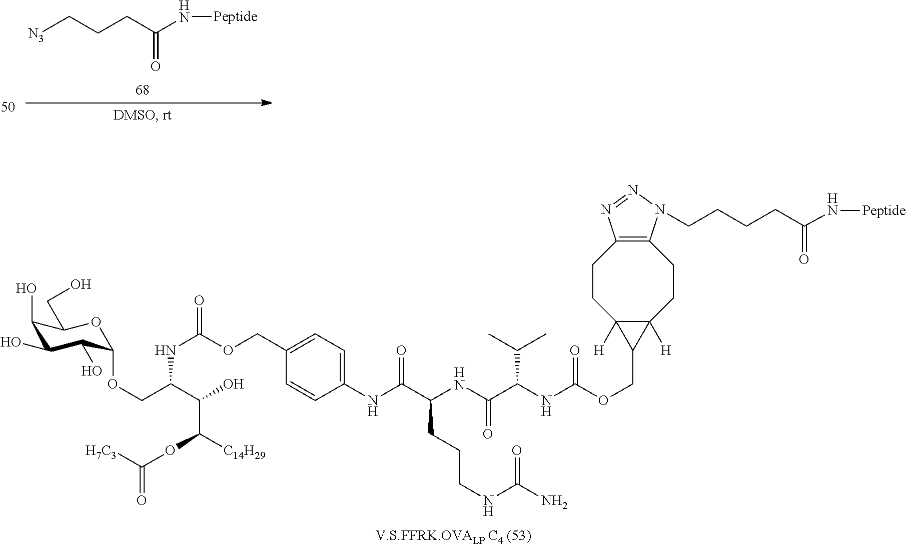

The cyclooctyne starting material 50 (2.0 mg, 0.0018 mmol) was coupled to the peptide following the general experimental method E. The conjugate 53 was purified (A:B—70:30 to 0:100 over 12 min) and obtained as a white coloured fluffy solid (6.14 mg, 65%, 99.9% pure by UV 254 nm, Rₜ=5.25 rains). HRMS-ESI—calculated for C₂₄₇H₃₈₉N₆₁O₇₀ [M+4H]⁴+1333.2211, observed 1333.2168.

V.S.FFRK.OVA_{LP} C₀ Conjugate (55)

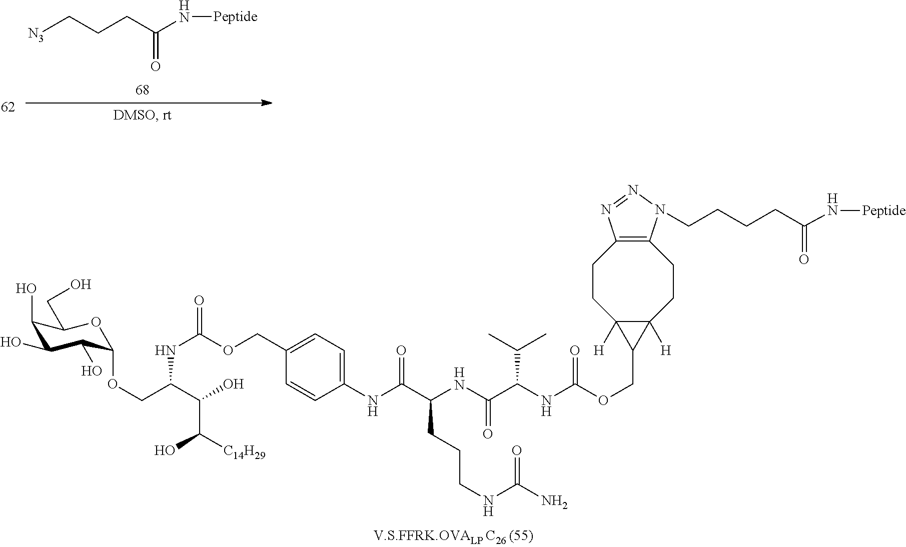

The cyclooctyne starting material 62 (2.0 mg, 0.0019 mmol) was coupled to the peptide following the general experimental method E. The conjugate 55 was purified (A:B—70:30 to 0:100 over 12 min) and obtained as a white coloured fluffy solid (4.91 mg, 49%, 99.9% pure by UV 254 nm, Rₜ=5.15 mins). HRMS-ESI—calculated for C₂₄₃H₃₈₃N₆₁O₆₉ [M+4H]⁴⁺ 1315.7106, observed 1315.7039.

### Example 5: Effect of Adjuvant on Vaccination to Increase Number of Liver T_{RM} Cells

### Adjuvant Effect is Unpredictable

B6 mice were vaccinated with a fusion protein consisting of an anti-Clec9A monoclonal antibody genetically fused to an antigenic peptide (NVFDFNNL—SEQ ID NO: 4). To generate immune responses with this fusion protein it is combined with an adjuvant (a compound that switches on the immune system). In these experiments, mice were injected with 50,000 PbT-I naïve malaria specific T cells and then vaccinated. 35 days later, mice were killed and the number and phenotype of PbT-I cells present in the spleen and liver determined. While combining this fusion protein with the adjuvant CpG as a vaccine led to induction of liver PbT-I T_{RM} cells (FIG. 1), combining this fusion protein with 5 alternative adjuvants failed to induce large numbers of PbT-I T_{RM} cells, despite inducing detectable circulating PbT-I memory T cells (as detected by response in the spleen). These results highlight the fact that adjuvants favouring induction of liver T_{RM} cells are rare, and that testing is required to determine if a given proposed adjuvant will in fact favour the induction of liver T_{RM} cells.

### Viral Vector Selection for Prime and Trap Methodology

It has been shown previously that combining fusion proteins as above with CpG adjuvant, used together with recombinant adeno-associated viral vector antigen expression in hepatocytes in the liver, though a complex method, leads to high numbers of liver T_{RM} cells. This approach is referred to as prime-and-trap. In this approach, the fusion protein and CpG prime T cells in the spleen, while the rAAV and CpG trap cells in the liver to form liver T_{RM} cells.

To test whether an alternative virus known to infect the liver might act as a vaccine for induction of liver T_{RM} cells, we expressed a malaria antigen (TRAP) in mouse cytomegalovirus (MCMV) and then tested whether infection with this virus would induce liver T_{RM} cells (FIG. 2).

As a positive control we used a prime-and-trap strategy with the same malaria antigen. We measured responses by flow cytometry using fluorescent peptide-loaded MHC tetramers that detect T cells that recognise this antigen. While the control prime-and-trap mice generated large numbers of liver T_{RM} cells, it was surprising to find that the MCMV-TRAP immunization did not, despite generating a good circulating T cell response as detected in the spleen.

α-GalCer alone does not produce high numbers of liver T_{RM} cells using Prime and Trap. As illustrated above, prime-and-trap vaccination using CpG as an adjuvant was very effective at inducing liver T_{RM} cells. To test whether CpG adjuvant might be substituted by α-GalCer, we compared prime-and-trap using CpG versus α-GalCer, (FIG. 3). C57BL/6 mice were injected with 50,000 naïve PbT-I T cells to track responses and were then vaccinated with the anti-Clec9A-NVY fusion protein together with either CpG or α-GalCer as adjuvant plus rAAV-NVY (SEQ ID NO: 15). Despite the abundance of NKT cells responsive to α-GalCer in the spleen and liver, this adjuvant did not produce high numbers of liver T_{RM} cells in the prime-and-trap setting. High numbers were, however, generated by our control adjuvant CpG known to induce T_{RM} cells in the prime-and-trap setting.

### Example 6: A Glycolipid-Peptide Vaccine Induces OVA-Specific Liver-Resident Memory CD8+ T Cells

C57BL/6 mice were adoptively transferred 40,000 naïve OT-I cells and then vaccinated with the glycolipid-peptide conjugate V.S.FFRK.OVA_{LP} that incorporates the migrated form of α-GalCer and a fusion peptide containing the I-Ab and H-2Kb epitopes of OVA (KISQAVHAAHAEINEAGRESIINFEKLTEWT) (SEQ ID NO: 11). This fusion peptide is designated as a “long peptide” (OVA_{LP}).

Upon uptake by dendritic cells, which are rich in cathepsin-mediated protease activity, the peptide and glycolipid moieties are released from the conjugate by cleavage of the linker, each moiety being made available for processing and loading onto MHC and CD1d molecules respectively. As a positive control for stimulation of CD8⁺ T cells, mice were vaccinated with a modified influenza A virus, PR8-OVA, which expresses the peptide sequence SIINFEKL (SEQ ID NO: 10)—the H-2Kb-binding epitope of OVA to which OT-I cells are restricted. An additional group of mice was vaccinated with unconjugated α-GalCer and the fusion peptide as an admixture.

Groups of vaccinated mice were harvested on days 21 and 60 to examine liver T_{RM} cell formation as assessed by staining for markers CD69 and CD62L.

FIG. 4A show how we identified T_{RM} cells (CD8⁺Ly5.1⁺ CD44⁺ CD69⁺ CD62L^{low}) and T_{EM} cells (CD8⁺Ly5.1⁺ CD44⁺ CD69⁻ CD62L^{low}) in the liver at day 21 after vaccination with V.S.FFRK.OVA_{LP} based on the gating strategy shown in FIG. 5. FIG. 4C shows that V.S.FFRK.OVA_{LP} was able to induce large numbers of OT-I liver T_{RM} cells by day 21 (approx. 10⁶ cells) and these cells persisted for more than 60 days (FIGS. 4C, F). These CD69+OT-I cells displayed a phenotype consistent with liver T_{RM} cells generated through other vaccination approaches, namely high expression of CXCR6, CD49a and CD101, with low expression of KLRG1 and CX3CR1 (FIG. 4B).

In contrast, neither PR8-OVA nor the unconjugated α-GalCer with peptide efficiently induced liver T_{RM} cells as can be seen from the number of T_{RM} cells present in the liver at day 21 post vaccination is shown in FIG. 4C. FIG. 4C shows the number of T_{RM}, T_{EM}, and T_{CM} (CD8⁺Ly5.1⁺ CD44⁺ CD69⁻ CD62L^{low}) cells present in the liver at day 21 post vaccination.

While an admixture of α-GalCer and the peptide was also poor at stimulating memory T cells in the spleen, both PR8-OVA and V.FFRK.OVA_{LP} induced OT-I effector and central memory (T_{EM} and T_{CM}) cells in this organ (FIGS. 4D, G). FIG. 4E shows the number of T_{RM}, T_{EM}, and T_{CM} cells present in the spleen at day 21 post vaccination and FIG. 4H shows the number of T_{RM}, T_{EM}, and T_{CM} cells present in the spleen at day 60 post vaccination.

Without wishing to be bound by theory, the inventors believe that both PR8-OVA (SEQ ID NO: 10) and V.S.FFRK.OVA_{LP} induce circulating memory T cells, but only V.S.FFRK.OVA_{LP} is efficient at inducing liver T_{RM} cells with FIG. 4F showing the number of T_{RM} cells present in the liver at day 60 post vaccination. Poor responses in mice primed with the admixture of α-GalCer and peptide also show that the chemical linkage of the two components is essential for efficient priming.

The results presented in FIGS. 4A-H are from two independent experiments using a total of 10 mice. Data displayed show mean±S.E.M and in some cases (C, F) data from individual mice. Groups in C and F were compared by one way ANOVA with Tukey's multiple comparison post-test. **** p<0.001.

### Example 7: Gating Strategy for Detection of Memory CD8+ T Cell Populations Examined in FIG. 4

With reference to FIG. 5, to identify liver Trm cells by flow cytometry in mice adoptively transferred with OT-I Ly5.1+ T cells, lymphocyte populations were gated by their low side scatter and broad forward scatter of laser light in upper left panel (SSA-A vs FSC-A). Single cells were examined and doublets excluded by gating on the diagonal of forward scatter height vs forward scatter amplitude (FSC-H vs FSC-A) in upper middle panel. Live cells were then selected by gating on low staining for propidium iodide (PI) thus excluding dead cells (upper right panel). Transferred OT-I cells that expressed Ly5.1 were then selected by gating on cells staining for Ly5.1 and lacking staining for Ly5.2 (lower left panel), the latter being expressed by recipient mice cells. OT-I cells were further selected by gating on those Ly5.1+ cells that expressed the T cell receptor molecule Va2 and the activation marker CD44 (lower middle panel). Finally, each memory OT-I cell population (T_{EM}, T_{RM} and T_{CM}), could be crudely identified by staining for CD62L and CD69 as follows: T_{EM} (CD62L−CD69−), T_{RM} (CD62L−CD69+) and T_{CM} (CD62L+CD69−) cells.

### Example 8: Prime and Boost Vaccination with a Glycolipid-Peptide Vaccine Induces Large Numbers of Plasmodium-Specific Liver T_{RM} Cells that Protect Against Liver-Stage Infection

As protection was suboptimal (9/12 mice) after one immunization with V.FFRK.NVY_{SP}, a booster immunization was employed in an attempt to increase liver T_{RM} cell generation and improve protection.

50,000 PbT-I.GFP cells were transferred into recipient B6 or CD1d^{-/-} mice. CD1d^{-/-} mice were treated with V.FFRK.NVY_{SP} at day 0 and 30 (Group 1). B6 mice were treated with V.FFRK.NVY_{SP} at day 30 only (Group 2), V.FFRK.NVY_{SP} at day 0 and 30 (Group 3), αClec9a-NVY and CpG at day 0 and V.FFRK.NVY_{SP} at day 30 (group 4), or with αClec9a-NVY and CpG (CC) at day 0 (Group 5) (FIG. 9). Organs were then harvested from mice from each group at day 50-60 and assessed for the generation of memory T cells. Liver and spleen memory T cells were examined (FIG. 9A-C). The number of liver PbT-I T_{RM} cells at day 50-60 post vaccination is shown in FIG. 9A. FIGS. 9B and 9C show the number of T_{RM}, T_{EM} and T_{CM} cells present in the liver (B) and spleen (C) at day 50-60 post vaccination.

This homologous boosting regimen using V.FFRK.NVY_{SP} induced an increase in PbT-I liver T_{RM} cells as well as increased memory T cells in the spleen compared to the alternative group of mice that received only a single dose at the booster stage (FIG. 9A-C). This shows the prime-boost regimen generated substantially higher T_{RM} cell numbers compared to single priming.

As an alternative, a heterologous, prime-boost vaccination method was employed where mice were vaccinated with CpG plus anti-Clec9A-NVY (without the virus used in P&T) and then boosted with V.FFRK.NVY_{SP}, or left un-boosted. This also resulted in a substantial increase in T_{RM} cells, showing that V.FFRK.NVY_{SP} was also very effective at boosting the CpG+anti-Clec9A-NVY induced primary responses. Of note, the dependence on iNKT cell help for expanding PbT-I responses after prime-boost vaccination with V.FFRK.NVY_{SP} was demonstrated by a lack of PbT-I cell expansion in CD1d^{-/-} mice (FIG. 9A-C).

To explore the extent of protection induced by these prime-boost regimens, mice were challenged with 200 P. berghei sporozoites (FIGS. 9D, E). The remaining mice in each group were challenged with 200 P. berghei sporozoites at day 73 and parasitemia was measured at day 79, 80, 81. Mice with two consecutive days of visible parasites in the blood were culled. Mice surviving challenge with low dose sporozoites were rechallenged with 3000 sporozoites. Parasitemia was measured at days 5, 6, 7, 8 and 12 post-high-dose-challenge. FIG. 9D shows the percentage of red blood cells containing parasites at day 7 post primary malaria challenge. This is 80 days after the start of the experiment.

Both prime-boost regimens induced sterile protection in all mice vaccinated. To further test the potential of these vaccination regimens, surviving mice were again challenged with a high dose of 3000 sporozoites (FIG. 9E). The number of mice that succumbed or were protected after 200, or 200 and 3000 sporozoite challenge is shown in FIG. 9E.

About half the mice from each group were protected, indicating very efficient immunization. Together, these data indicate that V.FFRK.NVY_{SP} can be used in prime-boost regimens and this vaccine induces large numbers of liver T_{RM} cells that efficiently protect against sporozoite challenge.

Results are from 3 independent experiments using at least 4 mice per group for each experiment. Data displayed show mean±S.E.M and in some cases (FIGS. 9A, D) data from individual mice. Groups in FIGS. 9A and D were compared by one way ANOVA with Tukey's multiple comparison post-test. Groups in FIG. 9E were compared using Fisher's exact test. *p<0.05, **p<0.01, *** p<0.001, **** p<0.0001.

### Example 9: Intermediate-Long Fatty Acyl Chains are Required for Optimal Liver T_{RM} Cell Formation and Protection from Malaria

As discussed above, while α-GalCer peptide conjugates are known to stimulate immune responses, the conjugates of the invention have been found to be particularly effective at increasing number of liver T_{RM} cells. One important feature of the conjugates of Formula I is the length of the fatty acyl chain, which needs to be at least C18. As shown in FIG. 6 liver NKT cell numbers were increased by C26, C24, C22, and C20 conjugates (FIG. 5D) and although all the compounds tested induced some level of activation of liver NKT cells the activation (as determined by down regulation of NK1.1 on NKT cells at the timepoint measured) was greater for the C26, C24, C22, C20 and C18 conjugates (FIG. 5E). A similar but less pronounced trend was observed for CD69 (FIG. 5F). Similar observations were observable in the spleen (Figures A-C).

As shown in FIG. 7, liver T_{RM} cells were efficiently induced by C26, C24, C22, C20 and C18 conjugates (FIGS. 7A, B). Values for C8 or less were ineffective at inducing liver T_{RM} cells. Total responses in the spleen are drastically reduced for C24 or less (FIG. 7C) suggesting full length FA chain length is important for good circulating T cell responses.

Consistent with the requirement for liver T_{RM} cells for efficient protection, fatty acid chains of C18 or greater induced protective immunity against challenge with sporozoites (FIGS. 7D and E). In this system, sporozoites expressed the antigen SIINFEKL (SEQ ID NO:10) (from the chicken ovalbumin protein) and responses were generated by conjugates that expressed this antigen.

### Example 10: A Glycolipid-Peptide Vaccine Induces Plasmodium-Specific Liver-Resident Memory CD8⁺ T Cells that Protect Against Liver-Stage Infection

V.S.FFRK.NVY_{SP} (containing a short peptide encompassing the NVYDFNLL (SEQ ID NO: 2) minimal epitope recognized by Plasmodium-specific CD8⁺ T cells from the PbT-I TCR transgenic line) was used to induce liver T_{RM} cells with specificity for the liver pathogen, Plasmodium berghei ANKA. The NYV_{SP} epitope is a peptide antigen mimic of the antigen recognized by PbT-I cells identified by a combinatorial peptide library approach. Use of this mimic was required because the authentic antigen for Plasmodium berghei ANKA was unknown. 50,000 PbT-I.GFP cells were adoptively transferred into recipient B6 mice. After one day, the recipient mice were treated with αClec9a-NVY/CpG (an established positive control), α-GalCer alone (αGC), or a glycolipid-peptide conjugate containing NVY short peptide [V.S.FFRK.NVY_{SP}]. Mice treated with αClec9a-NVY/CpG were also treated with rAAV-NVY at day 1 (i.e, by an established (positive control) prime-and-trap (P&T) vaccination regime consisting of two steps: first, mice were vaccinated with a combination of CpG oligonucleotide plus a Clec9A-specific monoclonal antibody (mAb) covalently linked to NVYDFNLL (SEQ ID NO: 2) on the heavy chain, and then the following day, mice were infected with a non-replicating recombinant adeno-associated virus that expresses the NVYDFNLL (SEQ ID NO: 2) epitope via the hepatocyte-specific α-1 antitrypsin promoter).

Examination of the livers of vaccinated mice on day 35 and assessment for generation of memory T cells by flow cytometry revealed that PbT-I liver T_{RM} cells were generated in response to both the P&T positive control, and V.FFRK.NVY_{SP}. FIG. 8A shows the number of T_{RM} cells (CD8⁺GFP⁺ CD44⁺ CD69⁺ CD62L^{low} KLRG1⁻) present in the liver at day 35 post vaccination. FIG. 8B shows the phenotype of T_{RM} and T_{EM} cells in the liver 35 days after vaccination with V.S.FFRK.NVY_{SP}. FIGS. 8C and 8D show the numbers of PbT-I T_{RM}, T_{EM} (CD44⁺ CD69⁻ CD62L^{low}), and T_{CM} (CD44⁺ CD69⁻ CD62L^{high}) cells in the liver (C) and spleen (D) at day 35 post vaccination.

The numbers observed in the V.S.FFRK.NVY_{SP} group were slightly lower (FIGS. 8A, C) than those in the P&T group. Liver T_{RM} cells generated through vaccination with a conjugate vaccine as described herein expressed high levels of CXCR6, CD49 and CD101, and as such were phenotypically identical to P&T generated T_{RM} cells (FIG. 8B). The numbers of liver T_{RM} cells observed using the malaria system were approximately 10-fold less than the OVA system and a similar trend in T_{EM} cell numbers was seen in the liver and spleen (FIGS. 2C, D). Although the numbers of liver T_{RM} cells observed using the malaria system were approximately 10-fold less than the OVA system and a similar trend in T_{EM} cell numbers was seen in the liver and spleen, the numbers observed were surprisingly high and of an order similar to prime and trap vaccination. Without wishing to be bound by theory the inventors' believe that these numbers will be capable of protecting against Plasmodium infection of the liver.

Given the capacity of liver T_{RM} cells to protect against malaria, the inventors then considered whether either αClec9a-NVY/CpG or V.S.FFRK.NVY_{SP} was able to induce immunity that was capable of sterile protection against challenge with sporozoites. Separate mice from the same groups as analyzed on day 35 above were challenged with 200 P. berghei ANKA sporozoites, the equivalent to one to two mosquito bites at day 42. As sporozoites grow in the liver for two days before leaving this site to enter the blood, liver-stage protection was measured by examining the blood for parasitemia from days 6-13 (FIGS. 8E, F). FIG. 8D shows the percentage of red blood cells infected with parasites at day 7 post malaria challenge. FIG. 8F shows the number of mice that succumbed or were protected after malaria challenge.

Nearly all mice vaccinated by the P&T control were protected from infection (13/14 mice). Importantly, a significant proportion (9/12) of mice were also protected after vaccination with V.S.FFRK.NVY_{SP}, whereas no mice were protected after receiving α-GalCer alone (FIG. 8F). These data demonstrate the efficacy of glycolipid-peptide conjugates as described herein for providing T_{RM} cell mediated protection against infective liver pathogens. Without wishing to be bound by theory the inventors believe that the data show that the V.S.FFRK.NVY_{SP} vaccine induced a sufficient number of T_{RM} cells for successful protection against malaria.

Results are from 2 or 3 independent experiments using at least 4 mice per group for each experiment, with the exception of the naïve group. Data displayed show mean±S.E.M and in some cases (A, E) data from individual mice. Groups in A and E were compared by one way ANOVA with Tukey's multiple comparison post-test. Groups in F were compared using Fisher's exact test. **** p<0.001.

### Example 11—Vaccination with Conjugate Vaccines Containing Epitope-Flanking Sequences Improves Liver T_{RM} Cell Generation

Utilizing a vaccine containing the minimal antigen-mimic epitope recognized by PbT-I T cells (that is NVYDFNLL) (SEQ ID NO: 2) was an efficient means to induce liver T_{RM} cell formation. However, the inventors made an additional surprising determination that induction of liver T_{RM} formation could be enhanced by adding a certain number of additional amino acid residues to either side of the epitope comprised in the peptide portion of a conjugate. Without wishing to be bound by theory, the inventors believe that addition of these residues protects the antigen from degradation and/or enhances the processing of the antigen. The “longer peptide” used in the P&T vaccination control described herein contains 4 flanking amino acid residues at either terminus from the protein antigen sequence (HSLSNVYDFNLLLERD) (SEQ ID NO: 12) and efficiently generates large numbers of liver T_{RM} cells.

To examine their theory, the inventors synthesized two new glycolipid-long peptide conjugates. V.S.NVY_{LP}, containing an N-terminal -AAA- spacer (analogous to the spacer used in our fusion protein-peptide) and V.S.FFRK.NVY_{LP}, containing the additional -FFRK- (SEQ ID NO: 1) proteasomal cleavage sequence.

Groups of C57BL/6 mice vaccinated with α-GalCer alone (α-GC), and the following conjugate compounds, V.S.FFRK.NVY_{SP}, V.S.FFRK.NVY_{LP} or V.S.NVY_{LP} (0.135 nmol each).

Organs were harvested from mice from each group at day 3 post vaccination and assessed for the expansion and activation of NKT cells by flow cytometry. FIGS. 11A and B show V.S.FFRK.NVY_{SP}, V.S.FFRK.NVY_{LP} or α-GalCer were, surprisingly, more efficient at expanding NKT cells than V.S.NVY_{LP}. FIGS. 11C-F show V.S.FFRK.NVY_{SP}, V.S.FFRK.NVY_{LP} or α-GalCer more potently activated NKT cells when compared to V.S.NVY_{LP}.

Groups of mice were adoptively transferred with 50,000 PbT-I T cells and then vaccinated the following day with α-GalCer alone (αGC), an admixture of α-GalCer and NVY long peptide (αGC+NVY_{LP}), and the following conjugate compounds, V.S.FFRK.NVY_{SP}, V.S.FFRK.NVY_{LP} or V.S.NVY_{LP}. Addition of V.S.NVY_{LP} allowed additional assessment of the requirement for the FFRK (SEQ ID NO: 1) sequence in the context of this longer peptide variant (FIG. 10).

Organs were harvested from mice from each group at days 21-35 post vaccination and assessed for the generation of memory T cells by flow cytometry. FIG. 10A shows the number of liver T_{RM} cells at days 21-35 post vaccination, while FIGS. 10B and C show the number of T_{RM}, T_{EM}, and T_{CM} cells present in the liver (B) and spleen (C) at days 21-35 post vaccination.

Analysis at day 21-35 revealed that V.S.FFRK.NVY_{LP} was most effective at inducing liver T_{RM} cells (FIGS. 10A and B). Similarly splenic T_{EM} and T_{RM} cell numbers were highest for this combination (FIG. 10C).

Liver T_{RM} cell numbers were lowest for the group of mice vaccinated with V.S.NVY_{LP}; i.e, lacking the FFRK (SEQ ID NO: 1) cleavage sequence. Early analysis (day 3) of iNKT cells after treatment revealed that V.S.NVY_{LP} generated the poorest increase in numbers in the liver or spleen of mice (FIGS. 11A and B, respectively), implying impaired activation of iNKT cells for conjugates lacking FFRK (SEQ ID NO: 1). This conclusion is supported by the reduced conversion of iNKT cells to NK1.1-negative in these mice and their poor downregulation of CD69 (FIG. 11C-F). This conclusion was further supported by the normal serum alanine aminotransferase (ALT) levels observed in these mice, contrasting with the raised levels seen for FFRK-containing vaccines (FIG. 11G).

The percentage of red blood cells infected with parasites at day 7 post-malaria challenge is shown in FIG. 10D, and the number of mice that succumbed or were protected after malaria challenge is shown in FIG. 10E.

To determine the level of protection the conjugate compounds provided, vaccinated mice were challenged with 200 P. berghei sporozoites at day 42 and parasitemia was measured by flow cytometry at days 6, 7, 8 and 13. Mice with two consecutive days of visible parasites in the blood were culled. Complete protection from sporozoite challenge was observed in all mice treated with V.S.FFRK.NVY_{SP} or V.S.FFRK.NVY_{LP} and about 50% of mice treated with V.S.NVY_{LP}, a result reflective of the liver T_{RM} cell numbers generated through vaccination (FIG. 10E). Combined, these data demonstrate the surprising increase in efficacy observed when using peptides with (a) an additional N-terminal cleavage sequence in conjugate vaccines and (b) flanking C and N terminal sequence, which together generate maximum numbers of liver T_{RM} cells, particularly in the context of providing protection from malaria.

Results are from two or three independent experiments using at least four mice per group for each experiment. Data displayed show mean±S.E.M and in some cases (FIGS. 10A, D) data from individual mice. Conjugate vaccine groups in FIGS. 10A and 10D were compared by one way ANOVA with Tukey's multiple comparison post-test. Groups in FIG. 10E were compared using Fisher's exact test. **p<0.01, *** p<0.001, **** p<0.0001.

### Example 12—Vaccination with Conjugate Vaccines Synthesized by Oxime Ligation Improves Liver T_{RM} Cell Generation

The efficacy of the oxime conjugates (V.Ox.G.J) was compared to that of the SPAAC conjugates (V.S.G.J) as follows. 50,000 PbT-I.GFP cells were transferred into recipient B6 mice. After one day, the recipient mice were treated with V.S.FFRK.NVY_{SP} or V.Ox.FFRK.NVY_{SP}. Organs were harvested from mice from each group at days 21 post vaccination and assessed for the generation of memory T cells by flow cytometry. The remaining mice per group were challenged with 200 P. berghei sporozoites at day 35 and parasitemia was measured by flow cytometry at days 6, 7, 8 and 13. Mice with two consecutive days of visible parasites in the blood were culled.

FIG. 12A shows the number of liver T_{RM} cells at days 21 post vaccination, while FIGS. 12B and 12C show the number of T_{RM}, T_{EM}, and T_{CM} cells present in the liver (B) and spleen (C) at day 35 post vaccination. The percentage of parasites present in red blood cells at day 7 post 200 sporozoite challenge is shown in FIG. 12D, and FIG. 12E shows the number of mice that succumbed or were protected after malaria challenge. In FIG. 12F the percentage of red blood cells infected with parasites at day 7 post 3000 sporozoite challenge is shown, while the number of mice that succumbed or were protected after malaria challenge is shown in FIG. 12G.

These results show that while all of the conjugates of the invention can be used as vaccines against hepatic disease, in particular malaria, the conjugates utilizing oxime linkers are particularly efficacious.

Results are from two independent experiments using at least four mice per group for each experiment (except FIGS. 12F and G). Data displayed show mean±S.E.M and in some cases (FIGS. 12A, D, F) data from individual mice. Conjugate vaccine groups in FIGS. 12A, D and F were compared by one way ANOVA with Tukey's multiple comparison post-test. Groups in FIGS. 12E and G were compared using Fisher's exact test. **p<0.01, *** p<0.001, **** p<0.0001.

### Example 13: Generation of NVFDFNNL-Specific Endogenous CD8 Memory T Cells by Glycolipid-Peptide Conjugate Vaccines

To this point, all data showing liver T_{RM} cell generation and protection using the glycolipid-peptide vaccination system have used mice with adoptively-transferred T cell receptor transgenic cells from either PbT-I mice or OT-I mice to monitor T cell responses. Thus, both monitoring of T_{RM} cell responses and the protection induced against sporozoite challenges has been aided by the artificial addition of naïve transgenic T cell specific for the sporozoites. To show that the conjugates of the invention could induce T_{RM} cells from a normal mouse T cell repertoire and that such vaccination could protect mice against sporozoites, we began studies using the actual malaria antigen recognised by PbT-I cells.

This antigen is from PBANKA 1351900 (60S ribosomal protein L6-2, putative) and has the following amino acid sequence: NVFDFNNL (SEQ ID NO:4). C57Bl/6 mice were vaccinated with a short peptide version V.Ox.FFRK.NVF_{SP} 3 times at 2 weekly intervals and then 3 weeks after the final boost were either enumerated for specific liver and spleen T cells (FIG. 13A) or challenged with 200 sporozoites (FIG. 13B). Vaccinated mice contained on average just over 300,000 liver T_{RM} cells specific for the vaccine epitope (as measured by tetramer staining) and were fully protected from sporozoite challenge. This experiment showed that the glycolipid-peptide conjugates of the invention could vaccinate a normal T cell repertoire to generate liver T_{RM} cells that could protect against malaria.

### Example 14: A Single Dose of the Conjugate Compound can Protect Mice from Malaria

While data in FIG. 13 was encouraging, it only tested mice vaccinated multiple times and it utilized a vaccine that we subsequently realised would be suboptimal as it only had the short peptide motif, with no surrounding sequence. To examine whether a vaccine with an extended peptide motif (i.e. AAASTNVFDFNNLS (SEQ ID NO: 5)) containing the NVFDFNNL (SEQ ID NO:4) epitope could induce liver T_{RM} cells and protect against sporozoite challenge using the normal T cell repertoire of a mouse, we vaccinated C57Bl/6 mice with VOx.FFRK.NFV_{LP} a single time then examined some mice for liver and spleen T cells specific for this epitope (day 35) or challenged mice with 200 PbA sporozoites on day 42 (FIG. 14). These data revealed that this conjugate generated around 250,000 liver T_{RM} cells specific for NVFDFNNL (SEQ ID NO:4) (FIG. 14A) and that vaccinated mice were fully protected from 200 sporozoites (FIG. 14B). To test further the level of protection, mice were again challenged on day 70 with 3000 sporozoites (FIG. 14B). This showed that most mice were also protected from this large challenge indicating a highly efficacious vaccine.

### Example 15: A Second Dose of the Conjugate Compound can Enhance Protection Malaria

Data shown in FIG. 9 reveal the potential to boost the PbT-I T cell response with a second dose of the vaccine. To examine whether a second dose of the vaccine could expand liver T_{RM} cell numbers generated from the endogenous T cell repertoire, and enhance protection from sporozoite challenge, we vaccinated C57Bl/6 mice with VOx.FFRK.NFV_{LP} at day 0 alone (NVF/-), day 30 alone (-/NVF), or at day 0 and 30 (NVF/NVF). These data revealed that a second dose of VOx.FFRK.NFV_{LP} generated over a million liver T_{RM} cells specific for NVFDFNNL (SEQ ID NO: 4) at day 60 compared to 250-500,000 from a single dose (FIG. 15A) and boosted responses in the spleen (FIG. 15B). To test further the level of protection, mice were challenged with 200 PbA sporozoites on day 66 and, if protected, rechallenged with 3000 sporozoites on day 85 (FIG. 15C). This showed that all mice receiving two doses were protected from both challenges indicating a highly efficacious vaccine.

### Example 16: Protection from a Single Dose of the Conjugate Compound is Maintained for 200 Days

While data in FIG. 14 was encouraging, it only tested mice at a single timepoint after vaccination (day 35-42). To examine the longevity of protection, we vaccinated C57Bl/6 mice with VOx.FFRK.NFV_{LP} a single time then examined some mice for liver T cells at various timepoints over 200 days or challenged mice with 200 PbA sporozoites at various times. These data revealed that liver T_{RM} cells specific for NVFDFNNL (SEQ ID NO: 4) have a half life of −425 days (FIG. 16A) and 90% of mice are protected up to 200 days after vaccination (FIG. 16B). This showed that VOx.FFRK.NFV_{LP} is a highly efficacious vaccine that provides long-term protection.

### Example 17: Protection from a Single Dose of the Conjugate Compound is Superior to the Current Gold-Standard Malaria Vaccine, Radiation Attenuated Sporozoites (RAS)

Data shown in FIGS. 14 and 16 reveal the effectiveness of the glycolipid-peptide vaccination system but these vaccines had yet to be compared to the current gold-standard malaria vaccine, radiation attenuated sporozoites (RAS). To make this comparison, we vaccinated C57Bl/6 mice with VOx.FFRK.NVF_{LP} or 50,000 RAS and challenged the mice a month later with 200 WT PbA sporozoites. Mice that succumbed to sporozoite challenge were killed and their livers assessed for the number of NVF-specific T_{RM} cells (FIG. 17A, grey circles) and the number of T_{RM} cells of any specificity (FIG. 17B, grey circles). Mice that did not become parasitemic were considered protected and their livers similarly analyzed at day 12 post-challenge (FIGS. 17A and B, open circles). These data revealed VOx.FFRK.NFV_{LP} vaccinated mice were better protected than RAS vaccinated mice (FIG. 17C) and NVF-specific liver T_{RM} cell numbers (FIG. 17A), and total liver T_{RM} cell numbers (FIG. 17B) were higher in VOx.FFRK.NVF_{LP} vaccinated mice. This experiment showed that a glycolipid-peptide conjugate vaccine is a superior vaccine to RAS.

### Example 18: Identification of the HLA-A*02:01-Restricted Epitope ILNSGLLAV (SEQ ID NO: 18) in P. falciparum RPL6 (PfRPL6 (PF3D7_1338200))

Having characterized PBANKA 1351900 (60S ribosomal protein L6-2, putative) RPL6 as a promising antigen in P. berghei, we sought to identify potential human-relevant antigens in its P. falciparum ortholog (PF3D7 1338200, also known as PF13 0213). To this extent, we used the Immune Epitope Database (IEDB: https://www.iedb.org/) epitope prediction resource (Vita, R. et al. 2019) to search for peptides within P. falciparum RPL6 that were capable of binding HLA-A*02:01; a common allele. This identified ILNSGLLAV (SEQ ID NO: 18) (PfRPL6₇₇₋₈₅) as a promising candidate. Immunization of HHD mice (Pascolo, S. et al., 1997), which express human HLA-A*02:01 and lack expression of murine MHC class molecules, with this peptide triggered an epitope-specific CD8⁺ T cell response (FIG. 18), raising its potential as a human antigen.

### Example 19: Generating a T_{RM} Response by Vaccination with a Conjugate Compound Comprising the HLA-A*02:01-Restricted Epitope ILNSGLLAV (SEQ ID NO: 18)

The epitope PfRPL6₇₇₋₈₅ (ILNSGLLAV) (SEQ ID NO: 18) is contained within the sequence of the gene PfRPL6 (PF3D7 1338200) as highlighted in bold below:

(SEQ ID NO: 21)
MTNTSNELKHYNVKGKKKVLVPVNAKKTINKKYFGRKVASKKKYVVQRK
LRKSIEVGKVAIILTGKHMGKRCIITKILNSGLLAVVGPYEINGVPLKR
VDSRYLVVTSTNIFNFENIAKLKDDFLNYAQDIDDDSFIKTLEIKKKQK
KLLKNKNEALFMNNVIDKIKEIRKEDPKVQKLEGIQKDIGSLLKPEILK
NKVFAHYLKSKFTLRNDMVLHKMKF.

We believe that the potential of this epitope, as a protective target antigen to be included in our vaccine, can be shown as follows. A skilled person in the art can generate a vaccine similar to VOx.FFRK.NVF_{LP} by substituting the above epitope and surrounding C- and N-terminal sequence for NVF_{LP}. In one example, flanking C- and N-terminal sequences surrounding this epitope could be from 1 to 4 amino acid residues, particularly 2 residues such as in TKILNSGLLAVVG (SEQ ID NO: 19). This conjugate vaccine prepared as described herein would then be used to vaccinate HHD mice as in Example 18. Examination of memory T cell responses in the liver can be carried out as described herein on day 35 using tetramer staining and flow cytometry to determine if this vaccine generated liver T_{RM} cells of the correct specificity. Following this, determinant of the protective capacity of the generated T_{RM} cells can be shown by challenging vaccinated HHD mice with recombinant P. berghei sporozoites expressing the PfRPL6. Preparation of these parasites is currently underway and is believed to be within the skill of those in the art.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art. Each of such external documents is also specifically incorporated by reference herein.

| AA Sequence | Designation | SEQ ID NO: |
|---|---|---|
| FFRK | | SEQ ID NO: 1 |
| NVYDFNLL | NVY_{SP} | SEQ ID NO: 2 |
| AAAHSLSNVYDFNLLLERD | NVY_{LP} | SEQ ID NO: 3 |
| NVFDFNNL | NVF_{SP} | SEQ ID NO: 4 |
| AAASTNVFDFNNLS | NVF_{LP} | SEQ ID NO: 5 |
| DNQKDIYYITGESINAVS | | SEQ ID NO: 6 |
| AAALTSALLNVDNLIQ | | SEQ ID NO: 7 |
| STNVFDFNNLS | | SEQ ID NO: 8 |
| SALLNVDN | | SEQ ID NO: 9 |
| SIINFEK | PR8-OVA | SEQ ID NO: 10 |
| KISQAVHAAHAEINEAGRESIINFEKLTEWT | Ova long peptide | SEQ ID NO: 11 |
| HSLSNVYDFNLLLERD | | SEQ ID NO: 12 |
| EIYIFTNI | | SEQ ID NO: 13 |
| LSNYVDFNLLLERD | | SEQ ID NO: 14 |
| AAV-NVY | | SEQ ID NO: 15 |
| FKFL | | SEQ ID NO: 16 |
| GFLG | | SEQ ID NO: 17 |
| ILNSGLLAV | PfRPL6 epitope | SEQ ID NO: 18 |
| TKILNSGLLAVVG | PfRLP6 epitope with flanking | SEQ ID NO: 19 |
| | residues (2) | |
| HSLSILNSGLLAVLERD | PfRLP6 epitope with flanking | SEQ ID NO: 20 |
| | residues (4) | |

### 7. INDUSTRIAL APPLICABILITY

The compounds, methods and uses of the invention find use for inducing an immune response in a subject that reduces infection, particularly hepatic infection, particularly malaria.

### REFERENCES

(n.d.).
Anderson R J, L. J. (2017). Augmenting Influenza-Specific T Cell Memory Generation with a Natural Killer T Cell-Dependent Glycolipid-Peptide Vaccine. ACS Chem Biol., 12(11), 2898-905.
Anderson, R. J. (2014). A self-adjuvanting vaccine induces cytotoxic T lymphocytes that suppress allergy. Nature Chemical Biology, 10(11), 943-949.
Caminschi I, P. A. (2008). The dendritic cell subtype-restricted C-type lectin Clec9A is a target for vaccine enhancement. Blood, 112(8), 3264-73.
Davies B, P. J. (2017). Cutting Edge: Tissue-Resident Memory T Cells Generated by Multiple Immunizations or Localized Deposition Provide Enhanced Immunity. J Immunol., 198(6), 2233-7.
Du, W. S.-H. (2007). Efficient, one-pot syntheses of biologically active alpha-linked glycolipids. Chem Commun (Comb), 23, 2336-2338.
Dubowchik, G. M. (2002). Cathepsin 6-labile dipeptide linkers for lysosomal release of doxorubicin from internalizing immunoconjugates: model studies of enzymatic drug release and antigen-specific in vitro anticancer activity. Bioconjug Chem, 13(4), 855-869.
Exley M A, B. N. (2003). Innate immune response to encephalomyocarditis virus infection mediated by CD1d. Immunology. Immunology, 110(4), 519-26.
Fernandez-Ruiz D, N. W. (2016). Liver-resident memory CD8⁺ T cells form a front-line defense against malaria liver-stage infection. Immunity, 45.
Fujii S, S. K. (2003). Activation of natural killer T cells by alpha-galactosylceramide rapidly induces the full maturation of dendritic cells in vivo and thereby acts as an adjuvant for combined CD4 and CD8 T cell immunity t. J Exp Med., 198(2), 267-79.
Giaccone G, P. C. (2002). A phase I study of the natural killer T-cell ligand alpha-galactosylceramide (KRN7000) in patients with solid tumors. Clin Cancer Res, 8(12), 3702-9.
Gonzalez-Aseguinolaza G, V. K. (2002). Natural killer T cell ligand alpha-galactosylceramide enhances protective immunity induced by malaria vaccines. J Exp Med., 195(5), 617-24.
Hermans I F, S. J. (2003). NKT cells enhance CD4+ and CD8+ T cell responses to soluble antigen in vivo through direct interaction with dendritic cells. J Immunol., 171(10), 5140-7.
Hogquist K A, J. S. (1994). T cell receptor antagonist peptides induce positive selection. Cell, 76(1), 17-27.
Holz L E, P. J. (2018). CD8(+) T Cell Activation Leads to Constitutive Formation of Liver Tissue-Resident Memory T Cells that Seed a Large and Flexible Niche in the Liver. Cell Rep., 25(1), 68-79 e4.
Khan T N, M. J. (2016). Local antigen in nonlymphoid tissue promotes resident memory CD8+ T cell formation during viral infection. J Exp Med., 213(6), 951-66.
Kimura K, K. D. (2013). CD8+ T cells specific for a malaria cytoplasmic antigen form clusters around infected hepatocytes and are protective at the liver stage of infection. Infect Immun., 81(10), 3825-34.
Krieg, A. (2006). Therapeutic potential of Toll-like receptor 9 activation. Nat Rev Drug Discov., 5(6), 471-84.
Lau L S, F.-R. D. (2014). CD8+ T cells from a novel T cell receptor transgenic mouse induce liver-stage immunity that can be boosted by blood-stage infection in rodent malaria. PLoS Pathog., 10(5), e21004135.
Li J, A. F. (2015). Antibodies targeting Clec9A promote strong humoral immunity without adjuvant in mice and non-human primates. Eur J Immunol., 45(3), 856-64.
Mackay L K, S. A. (2012). Long-lived epithelial immunity by tissue-resident memory T (T_{RM}) cells in the absence of persisting local antigen presentation. Proc Natl Acad Sci U.S.A., 109(18), 7037-42.
Pallett, L. J. (2017). IL-thigh tissue-resident T cells in the human liver: Sentinels for hepatotropic infection. The Journal of Experimental Medicine, 214(6), 1567-1580.
Pascolo, S., Bervas, N., Ure, J. M., Smith, A. G., Lemonnier, F. A., and Pe'rarnau, B. (1997)). HLA-A2.1-restricted education and cytolytic activity of CD8+ T lymphocytes from beta2 microglobulin (beta2m) HLA-A2.1 monochain transgenic H-2D^{b} beta2m double knockout mice. J. Exp. Med. 185, 2043-2051.
Ramakrishnan C, D. M. (2013). Laboratory maintenance of rodent malaria parasites. Methods Mol Biol., 923, 51-72.
Rodrigues M, N. R. (1993). The relative contribution of antibodies, CD4+ and CD8+ T cells to sporozoite-induced protection against malaria. Immunology, 80(1), 1-5.
Scheiblhofer S, L. J. (2017). Influence of protein fold stability on immunogenicity and its implications for vaccine design. Expert Rev Vaccines, 16(5), 479-89.
Schmidt N W, B. N. (2010). Extreme CD8 T cell requirements for anti-malarial liver-stage immunity following immunization with radiation attenuated sporozoites. PLoS Pathog., 6(7), e1000998.
Schmidt N W, P. R. (2008). Memory CD8 T cell responses exceeding a large but definable threshold provide long-term immunity to malaria. Proc Natl Acad Sci U.S.A., 105(37), 14017-22.
Seguin M C, K. F. (1994). Induction of nitric oxide synthase protects against malaria in mice exposed to irradiated Plasmodium berghei infected mosquitoes: involvement of interferon gamma and CD8+ T cells. J Exp Med, 180(1), 353-8.
Semmling V, L.-K. V. (2010). Alternative cross-priming through CCL17-CCR4-mediated attraction of CTLs toward NKT cell-licensed DCs. Nat Immunol., 11(4), 313-20.
Vita, R., Mahajan, S., Overton, J. A., Dhanda, S. K., Martini, S., Cantrell, J. R., Wheeler, D. K., Sette, A., and Peters, B. (2019). The Immune Epitope Database (IEDB): 2018 update. Nucleic Acids Res. 47, D339-D343
Weiss W R, S. M. (1988). CD8⁺ T cells (cytotoxic/suppressors) are required for protection in mice immunized with malaria sporozoites. Proc Notl Acad Sci U.S.A., 85(2), 573-6.
Widmann C, R. P. (1992). T helper epitopes enhance the cytotoxic response of mice immunized with MHC class I-restricted malaria peptides. J Immunol Methods, 155(1), 95-9.

1. A compound of Formula L 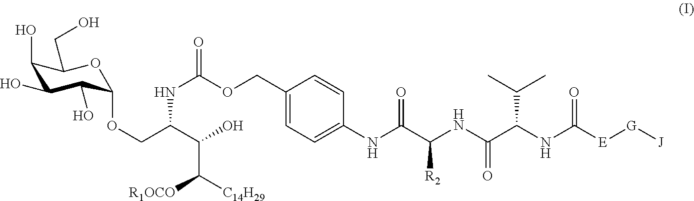
wherein
R₁ is (C₁₇-C₂₅)alkyl,
R₂ is the side-chain for alanine or citrulline,
E is a linker selected from S or Ox 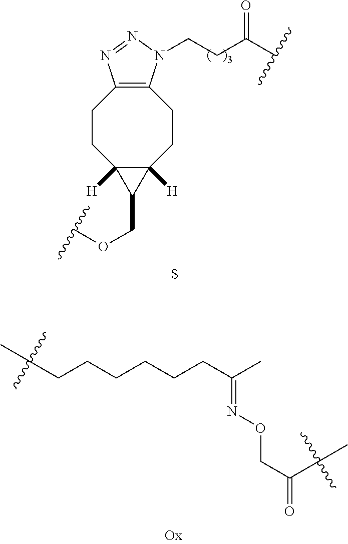
G is absent or is an amino acid sequence selected from the group consisting of FFRK (SEQ ID NO: 1), FKFL (SEQ ID NO: 16), and GFLG (SEQ ID NO: 17); and
J is a peptide antigen.

2. The compound of claim 1 which is a compound of Formula V.S.G.J: 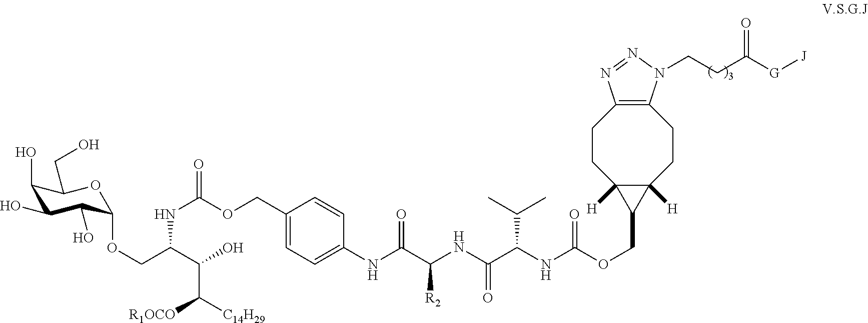
wherein
R₁ is (C₁₇-C₂₅)alkyl,
R₂ is the side-chain for alanine or citrulline,
G is absent or is the amino acid sequence selected from the group consisting of FFRK (SEQ ID NO: 1), FKFL (SEQ ID NO: 16) and GFLG (SEQ ID NO: 17); and
J is a peptide antigen.

3. The compound of claim 1 which is a compound of Formula V.Ox.G.J: 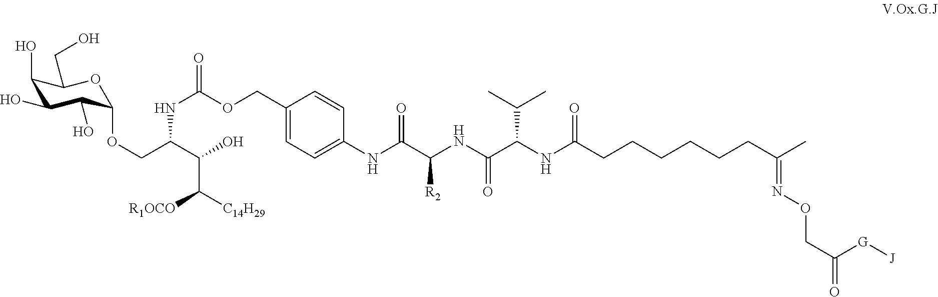
wherein
R₁ is (C₁₇-C₂₅)alkyl,
R₂ is the side-chain for alanine or citrulline,
G is absent or is the amino acid sequence selected from the group consisting of FFRK (SEQ ID NO: 1), FKFL (SEQ ID NO: 16) and GFLG (SEQ ID NO: 17); and
J is a peptide antigen.

4. The compound of claim 1, wherein R₁ is (C₁₉-C₂₅)alkyl.

5. The compound of claim 1, wherein R₂ is the side chain for citrulline.

6. The compound of claim 1, wherein G is FFRK (SEQ ID NO: 1).

7. The compound of claim 1, wherein J comprises an epitope that binds an antigen expressed by an organism that infects a subject's liver or at least one cell in the subject's liver.

8. (canceled)

9. The compound of claim 1, wherein J is selected from the group consisting of NVYDFNLL (SEQ ID NO: 2) (NVY_{SP}), AAAHSLSNVYDFNLLLERD (SEQ ID NO: 3) (NVY_{LP}), NVFDFNNL (SEQ ID NO: 4) (NVF_{SP}) and AAASTNVFDFNNLS (SEQ ID NO: 5) (NVF_{LP}), DNQKDIYYITGESINAVS (SEQ ID NO: 6), AAALTSALLNVDNLIQ (SEQ ID NO: 7), STNVFDFNNLS (SEQ ID NO: 8), EIYIFTNI (SEQ ID NO: 13), ILNSGLLAV (SEQ ID NO: 18), TKILNSGLLAVVG (SEQ ID NO: 19), and HSLSILNSGLLAVLERD (SEQ ID NO: 20).

10. A pharmaceutical composition comprising a compound of claim 1 and at least one pharmaceutically acceptable carrier or excipient.

11. (canceled)

12. A method of increasing the number of liver T_{RM} cells in a subject comprising administering to the subject a compound as defined in claim 1.

13. The method of claim 12, wherein the number of liver T_{RM} cells in the treated subject is increased relative to a control subject or relative to the number of liver T_{RM} cells in the subject before administration.

14. The method of claim 10, wherein the number of liver T_{RM} cells is increased relative to a control subject or relative to the number of liver T_{RM} cells in the subject before administration, by a number that is sufficient to provide at least some level of prophylaxis to the subject for at least 60 days.

15. A method of inducing an immune response that will reduce liver cell infection in a subject comprising administering to the subject a compound as defined in claim 1.

16. The method of claim 15, wherein the immune response reduces liver cell infection to the point of no on-going infection.

17. The method of claim 15, wherein the immune response prevents blood stage infection.

18. The method of claim 17, wherein blood stage infection is prevented for at least 60 days.

19. The method of claim 16, wherein the liver cell infection is a Plasmodium infection.

20. A method of treating or preventing malaria or hepatitis in a subject in need thereof, the method comprising administering to the subject a therapeutically effective amount of a compound as defined in claim 1.
